(19) United States
(12) Patent Application Publication
(10) Pub. No.: US 2026/0315600 A1
(43) Pub. Date: Oct. 15, 2026

(54) METHODS FOR STIMULATING AN IMMUNE RESPONSE

(71) Applicant: BEACON TECHNOLOGY SOLUTIONS INC., CHICAGO, IL (US)

(72) Inventors: BRIAN CLARK, CHICAGO, IL (US); Andrea Lee CLARK, CHICAGO, IL (US); Daniel Lawrence CLARK, HERMOSA BEACH, CA (US)

(21) Appl. No.: 19/668,509

(22) Filed: May 5, 2026

Related U.S. Application Data
(63) Continuation-in-part of application No. PCT/US2025/055651, filed on Nov. 14, 2025.
(60) Provisional application No. 63/721,238, filed on Nov. 15, 2024.
(60) Provisional application No. 63/736,389, filed on Dec. 19, 2024.
(60) Provisional application No. 63/767,458, filed on Mar. 5, 2025.
(60) Provisional application No. 64/008,153, filed on Mar. 17, 2026.

(51) Int. Cl. A61K 41/17; A61K 39/145; A61K 39/39; A61P 37/04

(52) U.S. Cl. CPC A61K 41/17; A61K 39/145; A61K 39/39; A61P 37/04

(57) ABSTRACT

Method of triggering an immune response in a subject, methods of preventing, treating or reducing a disease or a condition in a subject, methods for providing anti-pathogen immunity in a subject, methods for enhancing immunity in a subject, and methods for stimulating an immune response in a cell population or tissue in a subject are disclosed, the methods including the use of immunogenic compositions generated by far-ultraviolet (far-UVC) disinfection devices including a light source positioned within a housing and configured to emit a far-UVC light having an output wavelength of between about 206 nanometers to about 230 nanometers. There are one or more subject detection sensors positioned configured to detect the presence of one or more subjects and a controller in communication with the light source and the one or more subject detection sensors. The controller is configured to receive detection data from the one or more subject detection sensors and determine whether one or more subjects are within a range of the far-UVC light emitted by the light source. The controller is further configured to cause the light source to emit or cease emitting far-UVC light based on the amount of time one or more subjects are within the range of the emitted. far-UVC light.

### CROSS-REFERENCE TO RELATED APPLICATIONS

This application (i) claims priority to U.S. Provisional Patent Application No. 64/008,153, filed Mar. 17, 2026; and (ii) is a continuation-in-part of International Application No. PCT/US2025/055651, filed Nov. 14, 2025, which claims priority to U.S. Provisional Patent Application No. 63/721,238, filed Nov. 15, 2024, U.S. Provisional Patent Application No. 63/736,389, filed Dec. 19, 2024, and U.S. Provisional Patent Application No. 63/767,458, filed Mar. 5, 2025, each of which is incorporated by reference herein in its entirety.

### TECHNICAL FIELD

The present disclosure generally relates to light systems for disinfection and, in some embodiments, to an ultraviolet light disinfection system and method of use thereof, e.g., methods for stimulating an immune response.

### BACKGROUND

Vaccines are classified by the technology used to train the immune system, and the primary types of vaccines include live-attenuated, inactivated, subunit/conjugate, toxoid, mRNA, viral vector, and nucleic acid (DNA). They are stabilized with additives and optimized based on the administration method. Rigorous testing of vaccines is required to ensure safety, efficacy, and stability, enabling effective immunity stimulation. Aerosolized vaccine delivery and inhalation are emerging as delivery methods to stimulate mucosal immunity, particularly in the respiratory tract where many pathogens enter.

Despite these advancements, there remains an unmet need for safe and effective devices and methods for real-time environmental disinfection with the ability to produce polyvalent immunogenic compositions and methods to enhance immunity through environmental disinfection. This invention addresses this need.

### SUMMARY

In one aspect this disclosure provides an ultraviolet, such as, without limitation, a far-ultraviolet (far-UVC), disinfection device including a housing, a light source positioned within the housing and configured to emit a far-UVC light having an output wavelength of between about 100 nanometers to about 280 nanometers (e.g., 206 nanometers to about 230 nanometers), one or more subject detection sensors positioned within the housing and configured to detect the presence of one or more subjects, the one or more subjects consisting of one or more of human beings, one or more domesticated animals, and one or more farm animals or a combination thereof, and a controller optionally positioned within the housing and in communication with the light source and the one or more subject detection sensors. The controller is configured to receive detection data from the one or more subject detection sensors, determine, based on the received detection data, whether one or more subjects are within a range of far-UVC light emitted by the light source, in response to determining that a subject of the one or more subjects are within the range of the far-UVC light emitted by the light source for a predetermined amount of exposure time, cause the light source to cease emitting far-UVC light and in response to determining that no subject of the one or more subjects is within the range of far-UVC light emitted by the light source for the predetermined amount of exposure time, cause the light source to emit the far-UVC light.

In some embodiments, the one or more subject detection sensors includes at least one of an infrared sensor, a motion sensor, and a proximity sensor. In some embodiments, the predetermined amount of exposure time is less than or equal to an exposure limit for the subject being exposed to the far-UVC light emitted by the light source. In some embodiments, the controller is configured to cause the light source to cease emitting far-UVC light in response to the one or more subject detection sensors detecting the presence of a subject within the range of the emitted far-UVC light.

In some embodiments, the controller is configured to determine an effective disinfection rate based on an amount of time that the light source has been emitting far-UVC light. In some embodiments, the controller is configured to transmit the determined effective disinfection rate to a client device external to far-UVC disinfection device. In some embodiments, the one or more subject detection sensors includes one or more infrared sensors (e.g., about 2), one or more radar sensors, one or more lidar sensors, one or more cameras, and one or more motion sensors (e.g., about 4). In some embodiments, the controller is configured to cause the one or more subject detection sensors to activate at a predetermined detection interval, and when activated, the one or more subject detection sensors are configured to generate the detection data and transmit the detection data to the processor.

In some embodiments, the predetermined detection interval is less than or equal to one second. In some embodiments, the controller is configured to delay causing the light source to emit the far-UVC light in response to the one or more subject detection sensors detecting no subject within the range of the far-UVC light emitted by the light source by a predetermined amount of delay time. In some embodiments, the controller is configured to, in response to the light source emitting the far-UVC light continuously for a predetermined maximum emission amount of time, causing the light source to cease emitting or reducing intensity of the far-UVC light. In some embodiments, the predetermined maximum emission amount of time is about sixty minutes.

In some embodiments, the light source is configured to emit a far-UVC light having an output wavelength of about 222 nanometers. In some embodiments, the controller is configured to cause the light source to cease emitting far-UVC light in response to the one or more subject detection sensors detecting the presence of a subject within the range of the emitted far-UVC light for a threshold limit value (TLV) amount of time, wherein the TLV is based on the output wavelength of the emitted far-UVC light and/or the intensity of the far-UVC light. In some embodiments, the controller is configured to cause the light source to cease emitting far-UVC light in response to the one or more subject detection sensors detecting the presence of a subject within a predetermined distance of the light source 104. In some embodiments, the predetermined distance is about one inch to about six feet (e.g., three feet).

In another aspect, provided is a method of automatically disinfecting the air and surfaces within the range of a disinfection device, the method including causing one or more light sources, such as one or more light sources emitting UVA, UVB, UVC, and/or far-UVC light, disinfection device to emit light, where the disinfection device light source configured to emit the light, one or more subject detection sensors configured to detect the one or more subjects, the one or more subjects consisting of one or more of human beings, one or more domesticated animals, and one or more farm animals or a combination thereof, and a controller in communication with the output range of the light source and the one or more subject detection sensors and configured to selectively activate and deactivate each of the light source(s) and the one or more subject detection sensors. The method further includes in response to the one or more subject detection sensors detecting that a subject of the one or more subjects are within the range of the light emitted by the light source for a predetermined amount of exposure time, transmitting a deactivation signal from the controller to the light source to cause the light source to cease emitting or reduce the intensity of the light, and in response to the one or more subject detection sensors detecting no subject of the one or more subjects within the range of the light emitted by the light source, transmitting an activation signal from the controller to light source to cause light source to begin emitting or increase the intensity of the light.

In some embodiments, the method further includes receiving from a client device, external to the disinfection device, at the controller, an indication of a desired continuous activation period for the light source, the desired continuous activation period defined by a start time and a stop time, at the start time, transmitting from the controller to the light source the activation signal causing the light source to emit light, at a first time following the start time and prior to the stop time, determining via the one or more subject detection sensors that a subject is within range of the light emitted by the light source, and transmitting the deactivation signal from the controller to the light source to cause the light source to cease emitting the light, at a second time following the first time and prior to the stop time, determining that no subject is within the range of the light emitted by the light source, and transmitting the activation signal from the controller to the light source to cause the light source to begin emitting the light or increase its intensity, and at the end time, transmitting from the controller to the light source the deactivation signal causing the light source to cease emitting the light or decrease its intensity.

In one aspect, provided herein is a method of triggering an immune response in a subject. The method includes administering to the subject an immunogenic composition comprised in a volume of air and/or the surface area. In some embodiments, the immunogenic composition is generated by exposing the volume of air and/or the surface area to the disclosed disinfection system, which, in various embodiments, may utilize a UVA, UVB, or UVC, including without limitation, far-UVC, light source.

In one aspect, provided herein is a method of preventing, treating or reducing a disease or a condition in a subject in need thereof. The disclosed method includes administering to the subject a therapeutically effective amount of an immunogenic composition in a pharmaceutical acceptable carrier, where the immunogenic composition is prepared by the disclosed disinfection system.

In another aspect, provided herein is a method for providing anti-pathogen immunity in a subject in need thereof. The disclosed method includes administering to the subject a therapeutically effective amount of an immunogenic composition in a pharmaceutical acceptable carrier, where the immunogenic composition is prepared by the disclosed disinfection system.

In another aspect, provided herein is a method for enhancing immunity in a subject in need thereof. The disclosed method includes administering to the subject a therapeutically effective amount of an immunogenic composition in a pharmaceutical acceptable carrier, where the immunogenic composition is prepared by the disclosed disinfection system.

In yet another aspect, provided herein is a method for stimulating an immune response to a cell population or tissue in a subject in need thereof. The disclosed method includes administering to the subject a therapeutically effective amount of an immunogenic composition in a pharmaceutical acceptable carrier.

In some embodiments, the immunogenic composition of the disclosed methods include an inactivated pathogen and/or inactivated microbe. In some embodiments, the pathogen may include a microbe, a virus, a mold, a parasite, allergen, a fungi, a protozoa, a prion, and/or a bacteria.

In one aspect, provided herein is a pharmaceutical composition for preventing, treating or reducing a disease or a condition in a subject in need thereof. The disclosed pharmaceutical composition comprises an immunogenic composition in a pharmaceutical acceptable carrier, where the immunogenic composition is prepared by the disclosed disinfection system.

In another aspect, the disclosure provides a method of triggering an immune response in a subject, the method comprising administering to the subject an immunogenic composition comprised in a volume of air, a volume of liquid, and/or on a surface area. In some embodiments, the method further comprising generating the immunogenic composition by exposing the volume of air, a volume of liquid, and/or the surface area to a disinfection system, as disclosed herein, which, in various embodiments, may utilize a UVA, UVB, or UVC, including without limitation, far-UVC, light source. In another aspect, the disclosure provides method of preventing, treating or reducing a disease or a condition in a subject, the method comprising administering to the subject a therapeutically effective amount of an immunogenic composition prepared by a disinfection system disclosed herein. In another aspect, the disclosure provides a method for providing anti-pathogen immunity in a subject, the method comprising administering to the subject a therapeutically effective amount of an immunogenic composition prepared by a disinfection system disclosed herein. In another aspect, the disclosure provides a method for enhancing immunity in a subject, the method comprising administering to the subject a therapeutically effective amount of an immunogenic composition prepared by a disinfection system disclosed herein. In another aspect, the disclosure provides a method for stimulating an immune response in a cell population or tissue in a subject, the method comprising administering to the subject a therapeutically effective amount of an immunogenic composition prepared by a disinfection system disclosed herein. In some embodiments, the administering comprises a membrane contact, an eye contact, a skin-contact, and/or an inhalation. In some embodiments, the immunogenic composition comprises an inactivated pathogen. In some embodiments, the pathogen comprises a virus and/or a bacteria. In some embodiments, the immunogenic composition is comprised in a volume of air and/or on a surface area. In some embodiments, the immunogenic composition is generated by exposing the volume of air and/or the surface area to a disinfection system disclosed herein. In some embodiments, the immunogenic composition is comprised in a pharmaceutically acceptable carrier. In some embodiments, the methods further comprising optimizing the volume of inactivated pathogen in the volume of air to generate different levels of immunity. In some embodiments, the methods further comprising detecting the presence of pathogens to refine and optimize the use of a disinfection system disclosed herein and the dosage of inactivated pathogen received by a subject. In some embodiments, the methods further comprising considering people movements, structures, and interactions for optimizing the dosage of inactivated pathogen produced by a disinfection system disclosed herein. In some embodiments, the methods further comprising limiting the impact of new pathogens (e.g., viruses or bacteria) due to exposure to inactivated ones. In some embodiments, the methods further comprising generating immunogenic compositions based on multiple pathogens at the same time. In some embodiments, the methods further comprising generating immunogenic compositions based on new pathogens, without a traditional vaccine development process. In some embodiments, the immunogenic composition is polyvalent (e.g., provides protection against more than one pathogen). In some embodiments, a subject exposed to inactivated pathogens by a disinfection system disclosed herein lessens the impact of those pathogens if the subject is exposed again. In some embodiments, the level of immunity generated by a disinfection system disclosed herein may depend on the volume or number of a mix of live/inactivated pathogen in the air, with the inactivated pathogen being done by a disinfection system disclosed herein (i.e., combination immunity). In some embodiments, an optimal number of live/inactivated pathogens can be generated in the air that provokes an optimal immune response while also minimizing illness in a subject. In some embodiments, inhalation can occur via nasal cavity or mouth of a subject. In some embodiments, the volume of immunity generated may depend on the volume or number of the inactivated pathogen in the air that is inactivated by a disinfection system disclosed herein. In some embodiments, the methods disclosed herein further comprise use of a detection device to determine the volume of pathogen in the air for optimization of an immune response in a subject. In some embodiments, the methods disclosed herein further comprise measuring and optimizing the volume of inactivated pathogen in the air to generate different levels of immunity in a subject. In some embodiments, the methods disclosed herein further comprise detecting the presence of pathogens to refine and optimize the use of the device for pathogen inactivation. In some embodiments, the methods disclosed herein further comprise optimizing the dosage of inactivated pathogen received by a subject. In some embodiments, the methods disclosed herein further comprise optimization based on a given environment. In some embodiments, the methods disclosed herein further comprise accounting for environmental factors, e.g., people movements, structures, and interactions, for optimizing the dosage of inactivated pathogen generated by a disinfection system disclosed herein. In some embodiments, the methods disclosed herein further comprise generating sub-infectious level doses of an immunogenic composition (small volumes) which may confer some modest immunity even if not immediately measurable, thus creating a “priming” effect in a subject. In some embodiments, a subject is exposed to low doses. In some embodiments, the methods disclosed herein further comprise generating immunity either via single time exposure, or over time with multiple exposures over a sustained period of time, often dependent on the specifics of the environment. In some embodiments, a disinfection system disclosed herein may provide cross-reactive immunity to similar pathogens (e.g., common cold Coronaviruses may provide some protection to COVID-19). In some embodiments, a disinfection system disclosed herein generates immunity for as many pathogens as are present in the air at that time (e.g., if there are 5 viruses in the air that are inactivated, it could provoke immune responses for all at one time).

In other aspects, other embodiments are contemplated wherein the same beneficial inactivation of pathogens and triggering of an immune response can be achieved by exposing pathogens to variations of light treatments, such as visible light, UVA, UVB, UVC (including far-UVC or vacuum UVC). In one exemplary embodiment, a device may be provided having a light source located in an enclosure thereof. The device may be configured to circulate air through the enclosure containing the light source, and the light source may be used to inactivate pathogens as the air circulates through the enclosure. As the air source circulates through the spectrum of light that the device emits and exits the device, the inactivated pathogens are released in the surrounding environment and can trigger an immune response in a similar manner as described herein. The device may further be configured to utilize any of the sensors and other components described herein, such as pathogen detection sensors to detect the presence of pathogens in air as it circulates through the device and optimize the level of inactivated pathogens released into the surrounding environment. In some embodiments, the device may be configured to function in a duct, e.g., HVAC duct or water duct, wherein any frequency of light is shined on the air and/or water where it is thereafter breathed in and/or ingested (e.g., in an AC unit or in a water filter). In some embodiments, the device may be configured to function in a refrigerator unit against food-borne illnesses.

In certain aspects, the disclosure provides a method selected from: a method of triggering an immune response in a subject, a method of preventing, treating or reducing a disease or a condition in a subject, a method for providing anti-pathogen immunity in a subject, a method for enhancing immunity in a subject, and a method for stimulating an immune response in a cell population or tissue in a subject, the method comprising administering to the subject an immunogenic composition comprised in a volume of air, a volume of liquid, a volume of vapors, and/or on a surface area, wherein the immunogenic composition is generated by exposing a volume of air, a volume of liquid, a volume of vapors, and/or a surface area to an ultraviolet (UVC) disinfection system. In some embodiments, the cell population or the tissue comprise one or more of an organ, a mucus membrane, blood, and/or muscle. In some embodiments, the method is used: in an environment for immunity generation, whether real-time, delayed, or over weeks, months, years, etc. In some embodiments, the method is real-time, continuous, including an output of a device disclosed herein via air and surfaces as the modality. In some embodiments, dosage of an immunogenic composition can vary and impacts the amount of disinfection. In some embodiments, the ultraviolet (UVC) disinfection system comprises: a housing; a light source positioned within the housing and configured to emit a UVC light; a trigger subject detection sensor positioned within the housing and configured to detect the position of a subject and generate corresponding detection data, the trigger subject selected from a subject with an immune system, a mammal, a human being, an undomesticated animal, a reptile, an amphibian, a fish, a domesticated animal, and a farm animal; and a controller positioned within the housing and in communication with the light source and the trigger subject detection sensor, the controller configured to: receive detection data from the trigger subject detection sensor including position data of the subject; determine, based on the received detection data, whether the trigger subject is within a range of the UVC emitted by the light source; and based on the detection data, adjust an intensity of the UVC light. In some embodiments, the controller is further configured to, based on the position data, determine that a trigger subject is within range of the UVC light and reduce the intensity of the UVC light. In some embodiments, the controller is further configured to, based on the position data, determine that a trigger subject is not within range of the UVC light and increase the intensity of the UVC light. In some embodiments, the controller is further configured to adjust the intensity of the UVC light based on position data of the trigger subject and corresponding timestamps generated by the subject detection sensor. In some embodiments, the UVC disinfection system further comprising: a pathogen detection sensor in communication with the controller and configured to detect the presence of pathogens, and wherein the controller is configured to: receive pathogen detection data from the pathogen detection sensor; and in response to determining that one or more pathogens are detected within the range of the UVC light, cause the intensity of the UVC light emitted by the light source to be increased. In some embodiments, the controller is configured to determine an effective disinfection rate based on an amount of time that the light source has been emitting the UVC light. In some embodiments, the controller is configured to transmit the determined effective disinfection rate to a client device external to the UVC disinfection device. In some embodiments, the trigger subject detection sensor is a plurality of trigger subject detection sensors each in communication with the controller, the plurality of trigger subject detection sensors including one or more infrared sensors (e.g., about 2), one or more radar sensors, one or more lidar sensors, one or more cameras, and one or more motion sensors (e.g., about 4). In some embodiments, the controller is configured to cause the trigger subject detection sensor to activate at a predetermined detection interval, and wherein, when activated, the trigger subject detection sensor is configured to generate the detection data and transmit the detection data to the processor. In some embodiments, the controller is further configured to: in response to the light source emitting the UVC light continuously for a predetermined maximum emission amount of time, causing the light source to cease emitting the UVC light. In some embodiments, the controller is configured to cause the light source to cease emitting the UVC light in response to the trigger subject detection sensor detecting the presence of a trigger subject within the range or within a predetermined distance of the emitted UVC light for a threshold limit value (TLV) amount of time, wherein the TLV is based on the output wavelength of the emitted UVC light. In some embodiments, the step of administering an immunogenic composition comprises a skin-contact and/or an inhalation. In some embodiments, the immunogenic composition comprises an inactivated pathogen. In some embodiments, the pathogen comprises a virus, a microbe, a mold, and/or a bacteria. In some embodiments, a method of subject's exposure to an antigen could be through the eyes, mouth, skin, skin, skin, nose, ears, or other. In some embodiments, a pathogen (e.g., virus, microbe, etc.) can be inactivated by an UVC disinfection system disclosed herein through DNA damage, membrane damage, protein damage, partially inactivated, and/or fully exploded into smaller particles and pieces of itself. In some embodiments, the immunogenic composition is comprised in a volume of air, a volume of liquid, a volume of vapors, and/or on a surface area. In some embodiments, UVC comprises far-UVC. In some embodiments, UVC comprises any wavelength range or ranges of UVC (e.g., 200 nm to 280 nm). In some embodiments, the subject is a trigger subject.

In certain aspects, the disclosure provides a method disclosed herein, such as a method selected from: a method of triggering an immune response in a subject, a method of preventing, treating or reducing a disease or a condition in a subject, a method for providing anti-pathogen immunity in a subject, a method for enhancing immunity in a subject, and a method for stimulating an immune response in a cell population or tissue in a subject, the method comprising administering to the subject an immunogenic composition comprised in a volume of air, a volume of liquid, a volume of vapors, and/or on a surface area, wherein the immunogenic composition is generated by exposing a volume of air, a volume of liquid, a volume of vapors, and/or a surface area to an ultraviolet (UVC) disinfection system, such as any UVC disinfection system disclosed herein. One of ordinary skill in the art is aware of any number of methods to assess stimulation of an immune response. For example, ad ELISA or a Western blot may be used. In some embodiments, ELISA data supports that exposure to an UVC disinfection system disclosed herein generates inactivated substrates comprising microbes, microbial particles, or other immunogenic materials which thereafter induce an immune response in a subject. In some embodiments, a positive ELISA signal indicates the presence of antigen-specific antibodies directed against the administered antigen (e.g., microbes, fungi, protozoa, mold, parasites, allergens, prions, bacteria, and viruses such as influenza, coronavirus, and/or RSV), with signal magnitude reflecting a measurable, statistically significant increase relative to baseline, consistent with antigen recognition and antibody generation. In some embodiments, the observed antibody response is attributable to the administered UVC disinfection system-inactivated antigen and not to environmental or experimental confounders. In some embodiments, negative control groups, including (i) Day 0 baseline samples and (ii) subjects administered Beacon-treated saline, exhibit minimal to no detectable antigen-specific antibody signal. In some embodiments, control subjects are co-housed with antigen-exposed subjects, thereby controlling for potential environmental exposure (e.g., airborne particles, cage conditions, diet, or handling). In some embodiments, the absence of antibody generation in these controls demonstrates that immune activation requires direct exposure to an UVC disinfection system disclosed herein and is not induced by incidental environmental factors. In some embodiments, subjects intranasally exposed to an UVC disinfection system disclosed herein inactivated influenza or RSV exhibited detectable antigen-specific antibodies at multiple post-exposure time points, including Day 13 and Day 28. In some embodiments, antibody levels at Day 28 were greater than those observed at Day 13, indicating persistence and continued development of the immune response over time. In some embodiments, the presence of antigen-specific antibodies at these later time points is consistent with an adaptive immune response and supports the generation of immunological memory. In some embodiments, and without being bound by any particular theory, these data demonstrate that an UVC disinfection system disclosed herein mediated inactivation preserves sufficient antigenic structure to enable immune system recognition while eliminating infectivity, thereby enabling safe induction of an acquired immune response following exposure via non-invasive routes (e.g., intranasal administration).

In certain aspects, the disclosure provides a method disclosed herein, such as a method selected from: a method of triggering an immune response in a subject, a method of preventing, treating or reducing a disease or a condition in a subject, a method for providing anti-pathogen immunity in a subject, a method for enhancing immunity in a subject, and a method for stimulating an immune response in a cell population or tissue in a subject, the method comprising administering to the subject an immunogenic composition comprised in a volume of air, a volume of liquid, a volume of vapors, and/or on a surface area, wherein the immunogenic composition is generated by exposing a volume of air, a volume of liquid, a volume of vapors, and/or a surface area to an ultraviolet (UVC) disinfection system, such as any UVC disinfection system disclosed herein. In certain embodiments, the minimum effective concentration of an immunogenic composition capable of eliciting an immune response may be as low as a single antigenic unit. In some embodiments, an antigen may comprise a whole microorganism (e.g., virus, bacterium, or mold) or any immunogenic portion thereof (e.g., protein, peptide, nucleic acid fragment, membrane component, or structural fragment). In some embodiments, the minimum exposure required to initiate an immune response may include one (1) or more antigenic particles, fragments, or subcomponents thereof. In some embodiments, the minimum concentration may correspond to a single infectious unit equivalent (e.g., 1 plaque-forming unit (PFU) or 1 colony-forming unit (CFU)), or a fraction or component thereof that retains immunogenic properties. In some embodiments, a single antigenic exposure event may be sufficient to activate one or more immune cells, thereby initiating an immune response cascade. In some embodiments, a threshold for immune activation may vary depending on multiple factors, including but not limited to pathogen type, antigen structure, the subject's prior immune exposure, and route of administration. In some embodiments, the lower bound of the effective concentration is not limited to bulk dosing levels and may include minimal or unit-level exposures, including a single antigenic particle or fragment capable of immune recognition.

In certain aspects, the disclosure provides a method disclosed herein, such as a method selected from: a method of triggering an immune response in a subject, a method of preventing, treating or reducing a disease or a condition in a subject, a method for providing anti-pathogen immunity in a subject, a method for enhancing immunity in a subject, and a method for stimulating an immune response in a cell population or tissue in a subject, the method comprising administering to the subject an immunogenic composition comprised in a volume of air, a volume of liquid, a volume of vapors, and/or on a surface area, wherein the immunogenic composition is generated by exposing a volume of air, a volume of liquid, a volume of vapors, and/or a surface area to an ultraviolet (UVC) disinfection system, such as any UVC disinfection system disclosed herein. In certain embodiments, there is no fixed upper limit on the concentration of the immunogenic composition generated by the UVC disinfection system under normal operating conditions. In some embodiments, a maximum concentration of inactivated pathogens or antigenic material present in an environment is dependent on the amount and type of biological material present in that environment and the extent of ultraviolet exposure applied. In some embodiments, an UVC disinfection system may generate immunogenic compositions across a wide range of concentrations, including low, moderate, and high environmental loads, without a predefined upper threshold. In some embodiments, an effective “maximum” exposure is not limited by a requirement for intact organisms, as immunogenicity may arise from any quantity of antigenic material present, including highly modified or fragmented material. In some embodiments, an immunogenic composition may include any UV-modified form of a pathogen or particle that retains one or more epitopes sufficient to elicit an immune response, regardless of whether the structure fully resembles the original organism. In some embodiments, an UVC disinfection system disclosed herein can create as much as pathogen is in the current environment.

In certain aspects, the disclosure provides a method disclosed herein, such as a method selected from: a method of triggering an immune response in a subject, a method of preventing, treating or reducing a disease or a condition in a subject, a method for providing anti-pathogen immunity in a subject, a method for enhancing immunity in a subject, and a method for stimulating an immune response in a cell population or tissue in a subject, the method comprising administering to the subject an immunogenic composition comprised in a volume of air, a volume of liquid, a volume of vapors, and/or on a surface area, wherein the immunogenic composition is generated by exposing a volume of air, a volume of liquid, a volume of vapors, and/or a surface area to an ultraviolet (UVC) disinfection system, such as any UVC disinfection system disclosed herein. In certain embodiments, pathogen inactivation may occur across multiple media, including but not limited to air, surfaces, and liquids (e.g., water or other fluid environments). In some embodiments, the disclosed methods are not limited to a particular medium and may be applied in any setting in which microorganisms or particles are present. In some embodiments, the effectiveness of inactivation and/or subsequent immune activation may be influenced by environmental and physical parameters. In some embodiments, such parameters may include, but are not limited to: gravitational conditions (e.g., terrestrial or reduced gravity environments), altitude or elevation, atmospheric pressure, humidity, temperature, and the presence or variation of other environmental factors such as light, sound, or fluid dynamics. In some embodiments, these methods may be implemented in a wide range of environments, including enclosed or contained spaces, open-air settings, underwater environments, subterranean locations, or extraterrestrial (e.g., space) conditions. In some embodiments, characteristics of the exposure medium may further influence outcomes. In some embodiments, properties of surfaces (e.g., material composition, porosity, reflectivity, or texture) and properties of the surrounding air or fluid (e.g., particulate load, flow dynamics, or composition) may impact the degree of pathogen inactivation and/or the delivery of immunogenic material to a subject. In some embodiments, exposure of a subject to the resulting immunogenic composition may occur via one or more routes, including but not limited to inhalation (nasal or oral), ocular exposure, dermal contact, mucosal surfaces, or other biological interfaces capable of antigen uptake. In some embodiments, pathogen inactivation induced by the UVC disinfection system may occur through one or more mechanisms, including but not limited to nucleic acid damage, protein modification, membrane disruption, or structural fragmentation. In certain embodiments, microorganisms may be partially inactivated, fully inactivated, or fragmented into smaller particles or subcomponents, each of which may retain immunogenic properties. In some embodiments, the disclosed methods are adaptable across diverse environmental conditions, exposure routes, and inactivation states, thereby supporting the broad applicability of the claimed system for generating immunogenic compositions and inducing immune responses.

In certain aspects, provided herein is a method of triggering an immune response in a subject, the method comprising: administering to the subject an immunogenic composition, wherein the immunogenic composition is generated by exposing a volume of air, a volume of liquid, a volume of vapors, and/or a surface area to an ultraviolet light.

In some aspects, provided herein is a method of triggering an immune response in a subject, the method comprising: exposing a volume of air, a volume of liquid, a volume of vapors, and/or a surface area to an ultraviolet light to generate an immunogenic composition; and administering to the subject an immunogenic composition.

In some aspects, provided herein is a method of triggering an immune response in a subject, the method comprising: exposing a volume of air, a volume of liquid, a volume of vapors, and/or a surface area to an ultraviolet light to generate an immunogenic composition; and exposing the subject an immunogenic composition.

In some aspects, provided herein is a method for stimulating an immune response in a cell population or tissue in a subject, comprising: administering to the subject an immunogenic composition, wherein the immunogenic composition is generated by exposing a volume of air, a volume of liquid, a volume of vapors, and/or a surface area to an ultraviolet light.

In some aspects, provided herein is a method for stimulating an immune response in a cell population or tissue in a subject, comprising: exposing a volume of air, a volume of liquid, a volume of vapors, and/or a surface area to an ultraviolet light to generate an immunogenic composition; and administering to the subject an immunogenic composition.

In some aspects, provided herein is a method for stimulating an immune response in a cell population or tissue in a subject, comprising: exposing a volume of air, a volume of liquid, a volume of vapors, and/or a surface area to an ultraviolet light to generate an immunogenic composition; and exposing the subject an immunogenic composition.

In some aspects, provided herein is a method of preventing, treating, or reducing a disease or condition in a subject, the method comprising: administering to the subject an immunogenic composition, wherein the immunogenic composition is generated by exposing a volume of air, a volume of liquid, a volume of vapors, and/or a surface area to an ultraviolet light.

In some aspects, provided herein is a method of preventing, treating, or reducing a disease or condition in a subject, the method comprising: exposing a volume of air, a volume of liquid, a volume of vapors, and/or a surface area to an ultraviolet light to generate an immunogenic composition; and administering to the subject an immunogenic composition.

In some aspects, provided herein is a method of preventing, treating, or reducing a disease or condition in a subject, the method comprising: exposing a volume of air, a volume of liquid, a volume of vapors, and/or a surface area to an ultraviolet light to generate an immunogenic composition; and exposing to the subject an immunogenic composition.

In some aspects, provided herein is a method for providing anti-pathogen immunity in a subject, the method comprising: administering to the subject an immunogenic composition, wherein the immunogenic composition is generated by exposing a volume of air, a volume of liquid, a volume of vapors, and/or a surface area to an ultraviolet light.

In some aspects, provided herein is a method for providing anti-pathogen immunity in a subject, the method comprising: exposing a volume of air, a volume of liquid, a volume of vapors, and/or a surface area to an ultraviolet light to generate an immunogenic composition; and administering to the subject an immunogenic composition.

In some aspects, provided herein is a method for providing anti-pathogen immunity in a subject, the method comprising: exposing a volume of air, a volume of liquid, a volume of vapors, and/or a surface area to an ultraviolet light to generate an immunogenic composition; and exposing the subject an immunogenic composition.

In some aspects, provided herein is a method for enhancing immunity in a subject, the method comprising: administering to the subject an immunogenic composition, wherein the immunogenic composition is generated by exposing a volume of air, a volume of liquid, a volume of vapors, and/or a surface area to an ultraviolet light.

In some aspects, provided herein is a method for enhancing immunity in a subject, the method comprising: exposing a volume of air, a volume of liquid, a volume of vapors, and/or a surface area to an ultraviolet light to generate an immunogenic composition; and administering to the subject an immunogenic composition.

In some aspects, provided herein is a method for enhancing immunity in a subject, the method comprising: exposing a volume of air, a volume of liquid, a volume of vapors, and/or a surface area to an ultraviolet light to generate an immunogenic composition; and exposing the subject an immunogenic composition.

In some aspects, provided herein is a method of generating an immunogenic composition comprising exposing a volume of air, a volume of liquid, a volume of vapors, and/or a surface area to an ultraviolet light.

In some embodiments, the ultraviolet light comprises UVA, UVB, UVC, or any combination thereof. In some embodiments, the ultraviolet light comprises UVC. In some embodiments, the ultraviolet light comprises far-UVC. In some embodiments, the ultraviolet light comprises an output wavelength of about 254 nm or 254 nm. In some embodiments, the ultraviolet light comprises an output wavelength of from 315 nm to 400 nm. In some embodiments, the ultraviolet light comprises an output wavelength of from 280 nm to 315 nm. In some embodiments, the ultraviolet light comprises an output wavelength of from 100 nm to 280 nm. In some embodiments, the ultraviolet light comprises an output wavelength of from 200 nm to 280 nm. In some embodiments, the ultraviolet light comprises an output wavelength of from 230 nm to 280 nm. In some embodiments, the ultraviolet light comprises an output wavelength of from 200 nm to 230 nm. In some embodiments, the ultraviolet light comprises an output wavelength of from 206 nm to 230 nm. In some embodiments, the ultraviolet light comprises an output wavelength of about 222 nm or 222 nm. In some embodiments, the ultraviolet light is emitted from an ultraviolet (UVC) disinfection system.

In some embodiments, the volume of air comprises a suspended liquid particle. In some embodiments, the volume of air comprises a respiratory droplet. In some embodiments, the volume of air, volume of liquid, volume of vapors, and/or surface area comprises a particulate matter. In some embodiments, the particulate matter comprises a liquid particle. In some embodiments, the particulate matter comprises a respiratory droplet. In some embodiments, the volume of air, volume of liquid, volume of vapors, and/or surface area comprises a pathogen. In some embodiments, the volume of air, volume of liquid, volume of vapors, and/or surface area comprises a plurality of different pathogens. In some embodiments, the pathogen or the plurality of different pathogens are aerosolized.

In some embodiments, the pathogen is a microbe. In some embodiments, the pathogen is a bacteria, virus, mold, parasite, allergen, fungi, protozoa, or prion. In some embodiments, the plurality of different pathogens comprises two or more of a group consisting of a bacteria, virus, mold, parasite, allergen, fungi, protozoa, and prion. In some embodiments, the plurality of different pathogens comprises two or more different types or species of a bacteria, a mold, a parasite, allergen, a virus, a fungi, a protozoa or a prion.

In some embodiments, the immunogenic composition comprises an uncharacterized pathogen or uncharacterized plurality of different pathogens. In some embodiments, the pathogen is inactivated, attenuated, weakened, dormant, or killed after exposure to the ultraviolet light. In some embodiments, the pathogen is inactivated, attenuated, weakened, dormant, or killed pathogen or a particle thereof. In some embodiments, the inactivated, attenuated, weakened, dormant, or killed pathogen or a particle thereof comprises one or more epitopes for generating an immune response; optionally, wherein the immune response is against the pathogen. In some embodiments, the inactivated, attenuated, weakened, dormant, or killed pathogen or particle thereof is an aerosolized inactivated pathogen. In some embodiments, the dormant pathogen is viable but not infectious or not culturable.

In some embodiments, the volume of air, volume of liquid, volume of vapors, and/or surface area comprises at least from 10⁸ to 10¹¹ inactivated, attenuated, weakened, dormant, or killed virus or particles thereof. In some embodiments, the volume of air, volume of liquid, volume of vapors, and/or surface area comprises at least 5×10¹⁰ inactivated, attenuated, weakened, dormant, or killed virus or particles thereof. In some embodiments, the volume of air, volume of liquid, volume of vapors, and/or surface area comprises from 10⁸ to 10¹¹ inactivated, attenuated, weakened, dormant, or killed virus or particles thereof. In some embodiments, the volume of air, volume of liquid, volume of vapors, and/or surface area comprises 5×10¹¹ inactivated, attenuated, weakened, dormant, or killed virus or particles thereof.

In some embodiments, administering comprises contacting to skin, via inhalation, via intranasal, contacting to a membrane, or any combination thereof. In some embodiments, exposing the subject to the immunogenic composition comprises contacting to skin, via inhalation, via intranasal, contacting to a membrane, or any combination thereof. In some embodiments, the membrane is a mucus membrane. In some embodiments, the membrane is in the nasal cavity, mouth, or eye. In some embodiments, intranasal is via intranasal instillation.

In some embodiments, the subject comprises an immune system. In some embodiments, the subject is an animal. In some embodiments, the subject is a mammal, a reptile, an amphibian, or a fish. In some embodiments, the subject is an undomesticated animal. In some embodiments, the subject is a domesticated animal. In some embodiments, the subject is human.

In some embodiments, the immune response comprises generating an antibody against a pathogen. In some embodiments, the immune response is a humoral immune response. In some embodiments, the immune response is immune priming. In some embodiments, the immune response comprises inflammation. In some embodiments, the immune response comprises IL-8 production. In some embodiments, the disease or condition is associated with or resulting from a microbe. In some embodiments, the disease or condition is associated with or resulting from a bacteria, virus, mold, allergen, parasite, allergen, fungi, protozoa, and prion. In some embodiments, the disease or condition is RSV, influenza, or coronavirus.

In some embodiments, the cell population comprises an immune cell. In some embodiments, the cell population comprises a cell comprising a major histocompatibility complex. In some embodiments, the cell population comprises an antigen presenting cell. In some embodiments, the cell population comprises a dendritic cell, macrophage, B-cell, T-cell, NK cell, NKT cell, mast cell, basophil, granulocyte, innate lymphoid cell, endothelial cell, fibroblast, or any combination thereof. In some embodiments, the tissue is an organ, blood, muscle, mucus membrane, or any combination thereof.

In some embodiments, the ultraviolet light is emitted from an ultraviolet light system comprising a light source. In some embodiments, the ultraviolet light system comprises: a housing; and a light source that emits the ultraviolet light. In some embodiments, the ultraviolet light system comprises a trigger subject detection sensor. In some embodiments, the trigger subject detection sensor is positioned within the housing. In some embodiments, the trigger subject detection sensor is configured to detect the position of a subject. In some embodiments, the trigger subject detection sensor is configured to generate detection data on the position of a subject. In some embodiments, a controller is configured to receive detection data from the trigger subject including position data of the subject, determine, based on the received detection data, whether the trigger subject is within a range of the ultraviolet light emitted by the light source, and based on the detection data, adjust an intensity of the ultraviolet light. In some embodiments, the controller is further configured to, based on the position data, determine that a trigger subject is within range of the ultraviolet light and reduce the intensity of the ultraviolet light. In some embodiments, the controller is further configured to, based on the position data, determine that a trigger subject is not within range of the ultraviolet light and increase the intensity of the ultraviolet light. In some embodiments, the controller is further configured to adjust the intensity of the ultraviolet light based on position data of the trigger subject and corresponding timestamps generated by the subject detection sensor. In some embodiments, the ultraviolet light system further comprises a pathogen detection sensor. In some embodiments, the ultraviolet light system further comprises: a pathogen detection sensor in communication with the controller and configured to detect the presence of pathogens. In some embodiments, the controller is configured to: receive pathogen detection data from the pathogen detection sensor; and in response to determining that one or more pathogens are detected within the range of the ultraviolet light, causing the intensity of the ultraviolet light emitted by the light source to be increased. In some embodiments, the controller is configured to determine an effective disinfection rate based on an amount of time that the light source has been emitting the ultraviolet light. In some embodiments, the controller is configured to determine an effective pathogen inactivation rate based on an amount of time that the light source has been emitting the ultraviolet light. In some embodiments, the controller is configured to transmit the determined effective disinfection rate and/or the determined effective pathogen inactivation rate to a client device external to the ultraviolet light system. In some embodiments, the trigger subject detection sensor is a plurality of trigger subject detection sensors in communication with the controller. In some embodiments, the plurality of trigger subject detection sensors comprises two infrared sensors and four motion sensors. In some embodiments, the controller is configured to cause the trigger subject detection sensor to activate at a predetermined detection interval, and wherein, when activated, the trigger subject detection sensor is configured to generate the detection data and transmit the detection data to a processor. In some embodiments, the controller is further configured to: in response to the light source emitting the ultraviolet light continuously for a predetermined maximum emission amount of time, causing the light source to cease emitting the ultraviolet light. In some embodiments, the controller is configured to cause the light source to cease emitting the ultraviolet light in response to the trigger subject detection sensor detecting the presence of a trigger subject within the range or within a predetermined distance of the emitted ultraviolet light for a threshold limit value (TLV) amount of time, wherein the TLV is based on an output wavelength of the emitted ultraviolet light. In some embodiments, the subject is a trigger subject. In some embodiments, the ultraviolet light is emitted from an ultraviolet system, and wherein the ultraviolet system is configured to adjust an intensity or a targeted area of the ultraviolet light based on sensor data indicating the presence of pathogens in the volume of air, volume of liquid, volume of vapors, and/or surface area. In some embodiments, the intensity or the targeted area of the ultraviolet light is adjusted to alter the immunogenic composition.

In some embodiments, the ultraviolet light system is a UVA system, UVB system, UVC system, or any combination thereof. In some embodiments, the ultraviolet light system emits an output wavelength of from 315 nm to 400 nm. In some embodiments, the ultraviolet light system emits an output wavelength of from 280 nm to 315 nm. In some embodiments, the ultraviolet light system emits an output of from 100 nm to 280 nm. In some embodiments, the ultraviolet light system emits an output of from 230 nm to 280 nm. In some embodiments, the ultraviolet light system emits an output of from 200 nm to 230 nm. In some embodiments, the ultraviolet light system emits an output of from 206 nm to 230 nm. In some embodiments, the ultraviolet light system emits an output of about 254 nm or 254 nm. In some embodiments, the ultraviolet light system emits an output of about 222 nm or 222 nm.

In some embodiments, the ultraviolet light system is a UVC system, optionally, wherein the UVC system is a UVC disinfecting system. In some embodiments, the ultraviolet light system is a far-UVC system. In some embodiments, the ultraviolet light system is a 254 nm system that emits an output wavelength of about 254 nm or 254 nm. In some embodiments, the ultraviolet light system is a 222 nm system that emits an output wavelength of about 222 nm or 222 nm.

In an aspect, provided herein is an immunogenic composition produced by the process of exposing a volume of air, a volume of liquid, a volume of vapors, and/or a surface area to an ultraviolet light. In some embodiments, the ultraviolet light comprises UVA, UVB, UVC, or any combination thereof. In some embodiments, the ultraviolet light comprises UVC. In some embodiments, the ultraviolet light comprises far-UVC. In some embodiments, the ultraviolet light system is a UVA system, UVB system, UVC system, or any combination thereof. In some embodiments, the ultraviolet light system emits an output wavelength of from 315 nm to 400 nm. In some embodiments, the ultraviolet light system emits an output wavelength of from 280 nm to 315 nm. In some embodiments, the ultraviolet light system emits an output of from 100 nm to 280 nm. In some embodiments, the ultraviolet light system emits an output of from 230 nm to 280 nm. In some embodiments, the ultraviolet light system emits an output of from 200 nm to 230 nm. In some embodiments, the ultraviolet light system emits an output of from 206 nm to 230 nm. In some embodiments, the ultraviolet light system emits an output of about 254 nm or 254 nm. In some embodiments, the ultraviolet light system emits an output of about 222 nm or 222 nm. In some embodiments, the ultraviolet light comprises an output wavelength of about 222 nm or 222 nm. In some embodiments, the ultraviolet light is emitted from an ultraviolet (UVC) disinfection system.

In some embodiments, the volume of air comprises a suspended liquid particle. In some embodiments, the volume of air comprises a respiratory droplet. In some embodiments, the volume of air, volume of liquid, volume of vapors, and/or surface area comprises a particulate matter. In some embodiments, the particulate matter comprises a liquid particle. In some embodiments, the particulate matter comprises a respiratory droplet. In some embodiments, the volume of air, volume of liquid, volume of vapors, and/or surface area comprises a pathogen. In some embodiments, the volume of air, volume of liquid, volume of vapors, and/or surface area comprises a plurality of different pathogens. In some embodiments, the pathogen or the plurality of different pathogens are aerosolized.

In some embodiments, the pathogen is a microbe. In some embodiments, the pathogen is a bacteria, virus, mold, parasite, allergen, fungi, protozoa, or prion. In some embodiments, the plurality of different pathogens comprises two or more of a group consisting of a bacteria, virus, mold, parasite, allergen, fungi, protozoa, and prion. In some embodiments, the plurality of different pathogens comprises two or more different types or species of a bacteria, a virus, a mold, a parasite, allergen, a fungi, a protozoa or a prion. In some embodiments, the immunogenic composition comprises an uncharacterized pathogen or uncharacterized plurality of different pathogens. In some embodiments, the pathogen is inactivated, attenuated, weakened, dormant, or killed after exposure to the ultraviolet light. In some embodiments, the pathogen is inactivated, attenuated, weakened, dormant, or killed pathogen or a particle thereof. In some embodiments, the inactivated, attenuated, weakened, dormant, or killed pathogen or a particle thereof comprises one or more epitopes for generating an immune response; optionally, wherein the immune response is against the pathogen. In some embodiments, the inactivated, attenuated, weakened, dormant, or killed pathogen or particle thereof is an aerosolized inactivated pathogen. In some embodiments, the dormant pathogen is viable but not infectious or not culturable.

In some embodiments, the volume of air, volume of liquid, volume of vapors, and/or surface area comprises at least from 10⁸ to 10¹¹ inactivated, attenuated, weakened, dormant, or killed virus or particles thereof. In some embodiments, the volume of air, volume of liquid, volume of vapors, and/or surface area comprises at least 5×10¹⁰ inactivated, attenuated, weakened, dormant, or killed virus or particles thereof. In some embodiments, the immunogenic composition comprises at least from 10⁸ to 10¹¹ inactivated, attenuated, weakened, dormant, or killed virus or particles thereof. In some embodiments, the immunogenic composition comprises least 5×10¹⁰ inactivated, attenuated, weakened, dormant, or killed virus or particles thereof. In some embodiments, the immunogenic composition comprises from 10⁸ to 10¹¹ inactivated, attenuated, weakened, dormant, or killed virus or particles thereof. In some embodiments, the immunogenic composition comprises 5×10¹⁰ inactivated, attenuated, weakened, dormant, or killed virus or particles thereof. In an aspect, provided herein is a pharmaceutical immunogenic composition comprising: the immunogenic composition as described herein; and a pharmaceutically acceptable carrier and/or excipient.

In an aspect, provided herein is a method of triggering an immune response in a subject, the method comprising: exposing a volume of air, a volume of liquid, a volume of vapors, and/or a surface area to an ultraviolet light to generate an immunogenic composition; and exposing the subject to the immunogenic composition.

In some embodiments, the ultraviolet light comprises UVA, UVB, UVC, or any combination thereof. In some embodiments, ultraviolet light comprises UVC. In some embodiments, the ultraviolet light comprises an output wavelength of from about 100 nm to about 280 nm. In some embodiments, the ultraviolet light comprises far-UVC. In some embodiments, ultraviolet light comprises an output wavelength of from about 200 nm to about 230 nm. In some embodiments, the ultraviolet light comprises an output wavelength of about 222 nm.

In some embodiments, the ultraviolet light is emitted from an ultraviolet system, and wherein the ultraviolet system is configured to adjust an intensity or a targeted area of the ultraviolet light based on sensor data indicating the presence of pathogens in the volume of air, volume of liquid, volume of vapors, and/or surface area. In some embodiments, the intensity or the targeted area of the ultraviolet light is adjusted to alter the immunogenic composition.

In some embodiments, the volume of air, the volume of liquid, the volume of vapors, and/or the surface area comprises a suspended liquid particle, a respiratory droplet, a particulate matter, a pathogen, or a plurality of different pathogens. In some embodiments, the pathogen is a microbe, bacteria, virus, mold, parasite, allergen, fungi, protozoa, or prion. In some embodiments, the immunogenic composition comprises at least from 10⁸ to 10¹¹ inactivated, attenuated, weakened, dormant, or killed virus or particles thereof. In some embodiments, the pathogen or the plurality of different pathogens are inactivated, attenuated, weakened, dormant, or killed after exposure to the ultraviolet light. In some embodiments, the dormant pathogen or plurality of different pathogens are viable but not infectious or not culturable. In some embodiments, the inactivated pathogen or plurality of different pathogens are an aerosolized inactivated pathogen or an aerosolized inactivated plurality of different pathogens.

In some embodiments, the inactivated, attenuated, weakened, dormant, or killed pathogen or plurality of different pathogens comprises one or more epitopes for generating an immune response. In some embodiments, the immune response is against the pathogen or the plurality of different pathogens. In some embodiments, exposing the subject to the immunogenic composition comprises contacting to skin, via inhalation, via intranasal, contacting to a membrane, or any combination thereof. In some embodiments, the subject comprises an immune system.

In some embodiments, the method comprises preventing, treating, or reducing a disease or condition in the subject, wherein the disease or condition is associated with or resulting from a microbe.

Although various features of the aforementioned embodiments, as well as the embodiments discussed in detail below, are described in connection with one or more embodiments, it is also contemplated that one or more features described with respect to these various embodiments may be combined and utilized together as part of the same embodiment without necessarily departing the scope of the present invention.

### BRIEF DESCRIPTION OF THE DRAWINGS

The foregoing summary, as well as the following detailed description of embodiments of the ultraviolet disinfection device and method, will be better understood when read in conjunction with the appended drawings of exemplary embodiments. It should be understood, however, that the invention is not limited to the precise arrangements and instrumentalities shown.

In the drawings:

FIG. 1 is a block diagram of an ultraviolet disinfection system in accordance with an exemplary embodiment of the present invention;

FIG. 2 is perspective view of the ultraviolet disinfection system of FIG. 1;

FIGS. 3A-3C are use case diagrams illustrating aspects of the present disclosure;

FIGS. 4A-4D are use case diagrams illustrating aspects of the present disclosure;

FIGS. 5A-5B are use case diagrams illustrating aspects of the present disclosure;

FIGS. 6A-6C are use case diagrams illustrating aspects of the present disclosure;

FIGS. 7A-7D are use case diagrams illustrating aspects of the present disclosure; and

FIG. 8 is a block diagram of an ultraviolet disinfection system in accordance with another exemplary embodiment of the present invention.

FIG. 9A depicts a continuous Far-UVC 222 nm disinfection system and a set-up for virus inactivation. FIG. 9B depicts a continuous Far-UVC 222 nm disinfection system and a set-up for virus inactivation (End Time (T8): 5:55 PM EST).

FIG. 10A depicts a continuous Far-UVC 222 nm disinfection system user interface set-up for virus inactivation. FIG. 10B depicts a continuous Far-UVC 222 nm disinfection system user interface set-up for virus inactivation (Start Time (T0): 9:55 AM EST).

FIG. 11 depicts binding titers to INFV A/PR/8/34 as measured by ELISA for three mice after exposure to Beacon light-inactivated INFV A/PR/8/34 for each serum sample dilution as compared to background at Day 14 (FIG. 11A) and Day 28 (FIG. 11B). Error bars represent standard deviation.

FIG. 12 depicts ELISA binding profiles for Day 14 and Day 28 serum samples from mice after exposure to Beacon light-inactivated INFV A/PR/8/34 (FIG. 12A) and a comparison of EC₅₀ values (half-maximal Effective Concentration) which were derived from the ELISA binding profiles (FIG. 12B) using 4-parameter logistic regression in GraphPad Prism.

FIG. 13 depicts the ELISA signal over time after exposure to RSV, INFV A/PR/8/34, or saline (e.g., 1×DPBS control) in mice (FIG. 13A) as well as the average fold change in ELISA signal compared to the signal prior to administration of Beacon light-inactivated virus (FIG. 13B).

### DETAILED DESCRIPTION

Ultraviolet germicidal irradiation (UVGI) is one of many disinfection methods that use Ultraviolet (UV) light to kill or inactivate microorganisms by destroying nucleic acids and disrupting their DNA, leaving them unable to perform vital cellular function. However, existing and/or commercially available UVGI devices emit a UV light that is unsafe to subjects such as human beings and animals such as domesticated animals and farm animals. For example, the wavelength of the UV light emitted by such devices can cause permanent damage to subjects within a very short time frame (e.g., within a couple of seconds) of being exposed to said UV light. Put another way, the UV light emitted by such devices is nearly immediately harmful to the subjects outlined above.

Additionally, there is a need to effectively disinfect the air and various surfaces within a given indoor or outdoor public or private space. For example, it is difficult to effectively kill or inactivate surfaceborne or airborne pathogens, such as, but not limited to, viruses, molds, parasites, allergens, bacteria and/or air pollutants (e.g., allergens or other chemicals) that exist within homes and other public or private spaces. As such, there is a need to provide an ultraviolet disinfection device, which emits a safe UV light (e.g., a 222 nm wavelength UV light) to disinfect the air and surfaces within a public or private space, and which is safe to operate around subjects such as human beings, domesticated animals (e.g., household pets) and/or farm animals.

Inhaled and aerosolized vaccines are emerging as approaches for stimulating mucosal immunity, targeting the respiratory tract where many pathogens enter. These vaccines—including live attenuated, viral-vectored, inactivated, and nucleic acid-based formulations—have shown promise in enhancing local IgA secretion, resident memory T cells, and systemic immunity. Studies also demonstrate that controlled exposure to inactivated or weakened airborne pathogens can prime immune responses without causing infection, similar to the concept of sub-infectious dosing in natural immunity.

Pathogens inactivated by 222 nm far-UVC that are subsequently inhaled by occupants can help those individuals develop immunity from a wide variety of pathogens, at a variety of dosage levels range from infectious level to sub infectious levels. The use of far-UVC (or UV more broadly) to inactivate pathogens and intentionally provoke an immune response is referred to as “The Rimoldi Effect.” The “Rimoldi Effect” may be conferred across multiple pathogens concurrently providing multivalent protection.

This disclosure provides the intersection of far-UVC pathogen inactivation and the benefits of inhaled vaccines, highlighting that inactivated airborne pathogens from far-UVC, even at sub-infectious dose levels, can modulate immune memory and mucosal defense mechanisms. The disclosed cross-section of far-UVC environmental disinfection and the immune-boosting nature of vaccines provides a cutting-edge new technology to help enhance population immunity.

The human body's ability to respond to many antigens at once and the “Rimoldi Effect” is applicable multivalently, across many pathogens concurrently, and dependent on the presence of inactivated pathogen in the room.

Numerous details are described herein in order to provide a thorough understanding of the example embodiment illustrated in the accompanying drawings. However, some embodiments may be practiced without any of the specific details, and the scope of the claims is only limited by those features and aspects specifically recited in the claims. Furthermore, well-known methods, components, and circuits have not been described in exhaustive detail so as not to unnecessarily obscure pertinent aspects of the embodiments described herein.

Aspects of the present invention are described with reference to the safety of an emitted UV light in relation to subjects (e.g., human being, domesticated animal, farm animal) exposed to the UV light. The “safety” of the UV light may refer to an amount of time needed to cause damage to the subject in response to the subject being exposed to the UV light. Damage to a subject from an emitted UV light may include, but is not limited to, skin damage (e.g., sunburn), damage to the subjects DNA, inflammation of the subject's cornea, temporary or permanent vision impairment, blindness, DNA lesions, erythema, and/or photo-keratitis. In some embodiments, the amount of time needed to cause damage to the subject may refer to the threshold limit value (TLV) for UV exposure. The TLV for UV exposure may be dictated by one or more existing trade organizations or standards organizations such as, but not limited to, the American Conference of Governmental Industrial Hygienists (ACGIH) or Illuminating Engineering Society (IES). The TLV may be calculated based on the output wavelength of the UV light, distance of a subject from the origin point of the UV light (e.g., a light source), an amount of time a subject is exposed to the UV light, the frequency at which a subject is exposed to the UV light, and/or the strength of the UV light (e.g., amount of radiation released by the UV light). according to means known to those skilled in the art. The TLV may represent the maximum allowable time a subject may be continuously exposed to a UV light before adverse effects or damage is caused to the subject. In some embodiments, a UV light having an associated TLV of greater than or equal to about 23 mJ/cm² may be considered safe whereas a UV light having an associated TLV of less than 23 mJ/cm² may be considered unsafe. In some embodiments, a UV light may be considered “safe” for purposes of this disclosure, if the UV light does not cause adverse effects and/or damage to a subject after being continuously exposed to the UV light for about at least six minutes, ten minutes, twelve minutes, or fifteen minutes.

A subject, as referenced herein, may be a group consisting of one or more of human beings, one or more domesticated animals, and/or one or more farm animals. For example, a human infant, child, adolescent and/or adult would be considered a subject for purposes of this disclosure whereas a fly may not. Further to this example, domesticated animals may refer to any animal commonly kept as a pet in family households in the United States, including, but not limited to dogs, cats, guinea pigs, rabbits and hamsters; and any animals commonly kept for companion or commercial purposes. It should be understood that domesticated animals may include amphibians, reptiles, and certain insects that human beings may keep as pets. A farm animal may refer to an animal farmed commercially for its meat, its skin or anything else produced by it (e.g., cows, pigs, sheep).

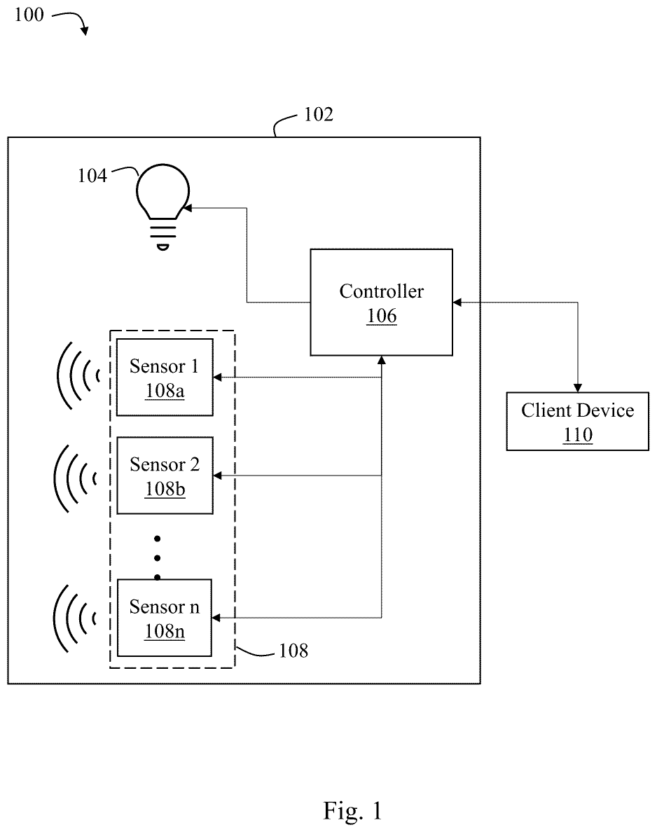

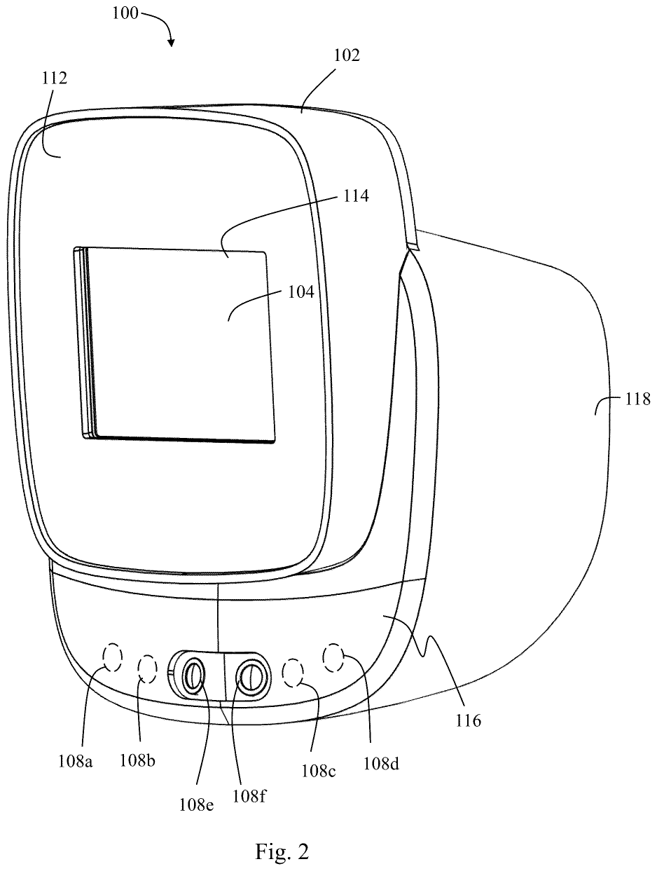

Referring to the drawings in detail, wherein like reference numerals indicate like elements throughout, there is shown in FIGS. 1-2 an ultraviolet disinfection system, generally designated 100, in accordance with an exemplary embodiment of the present invention. The UV system 100 may be configured to be positioned within a public or private space and safely emit a UV light that disinfects the air and/or surfaces within the emission range of the UV light.

Referring to FIGS. 1-2, the UV system 100 may include a light source 104, a controller 106, and one or more subject detection sensors 108. The controller 106 may include one or more processors configured and/or non-transitory computer readable storage mediums. The controller 106 may be in communication with the light source 104, one or more sensors (e.g., subject detection sensors 108, pathogen detection sensors), and/or other data sources (e.g., environmental data sources, weather data sources, public health report data, wastewater tracking data, geographical location data, etc.) such that data may be transmitted to and from the controller 106. In some embodiments, the controller 106 is configured to control operation of the light source 104 based on data received from one or more of the sensors and/or other data sources, as discussed in more detail below. In some embodiments, the controller 106 is configured to optimize operation of the light source 104 based on a plurality of different data points. For example, the controller 106 may be configured to optimize the intensity of a UV light emitted by the light source 104 based on detection data relating to the detection of subjects and/or detection data relating to the detection of pathogens within an area. In some embodiments, the controller 106 is configured to selectively increase and/or decrease the intensity of a UV light emitted by the light source 104.

In some embodiments, the light source 104, controller 106, and/or subject detection sensors 108 are coupled to and/or positioned within a housing 102 such that the system 100 is included in a device (e.g., a UV device). In some embodiments, the light source 104, controller 106, and/or the subject detection sensors 108 are not contained within a common housing and are communicably coupled to one another (e.g., via local area network (LAN), wide area network (WAN) or any other wireless or wired communications means). For sake of brevity, aspects of the present disclosure will be described in relation to the system 100 incorporated into a common housing 102, however it should be understood that elements of the system 100 may exist outside of a common housing.

The controller 106 may be in communication with the light source 104 such that the controller 106 may selectively activate and deactivate the light source 104. The controller 106 may also be in communication with the one or more subject detection sensors 108 such that the controller 106 may determine whether a subject is within the range of the UV light emitted by the light source 104. In some embodiments, the UV system 100 may include one or more light sources 104 each in communication with the controller 106. For sake of brevity, the UV system 100 will be described with reference to a single light source 104. In some embodiments, the UV system 100 may be a far-UVC disinfection system 100 configured to emit a far-UVC light (e.g., the light source 104 may be a far-UVC light source configured to emit a far-UVC light).

The controller 106 may include one or more processors and/or one or more memory units configured to store and execute executable code for controlling operations of the UV system 100 discussed herein. For example, the controller 106 may be in communication with the one or more subject detection sensors 108 and/or the light source 104 to control operation thereof. In some embodiments, the controller 106 includes a substrate (e.g., a printed circuit board (PCB)) having electrically connected thereto one or more memory devices (e.g., read-only memory (ROM), flash memory, dynamic random-access memory (DRAM), or static memory), and a processing unit (e.g., a processor, a microprocessor, an application specific integrated circuit (ASIC), or the like).

In some embodiments, the UV system 100 may include a power source (not shown) electrically connected to the light source 104, the controller 106, the one or more subject detection sensors 108 and/or any other electrically powered components coupled to the housing 102. The power source may be a battery pack configured to receive one or more batteries. In other embodiments, the power source may be a rechargeable power source. In other embodiments, the power source is electrically connected to a power cable to enable a user to plug the cable into an outlet connected to an electrical grid in the user's home.

In some embodiments, the controller 106 is configured to control an intensity of the far-UVC light emitted by the light source 104. The controller 106 may be configured to adjust an amount of power (e.g., a wattage) supplied to the light source 104 such that the intensity of the far-UVC light generated therefrom is adjusted. For example, the controller 106 decreases a wattage supplied to the light source 104 to decrease the intensity of the far-UVC light emitted therefrom and increases the wattage to increase the intensity of the far-UVC light. In some embodiments, intensity when referring to the UV light (e.g., far-UVC light) emitted by the light source 104 may refer to the amount of UV radiation energy per unit area per second (e.g., milliwatts per square centimeter). In some embodiments, the controller 106 is configured to adjust the intensity of the light emitted by the light source 104 based on data received from the sensors 108 and/or one or more other measurement or sensing devices in communication with the controller 106, as discussed in more detail below. In some embodiments, the controller 106 is configured to adjust intensity of the UV light based on detection of pathogens within the range of the light source 104.

In some embodiments, the UV system 100 is configured to increase the intensity of UV in response to detecting a subject. The controller 106 may be configured to increase the intensity of a far-UVC light emitted by the light source 104 in response to receiving a signal from the subject detection sensors 108. In some embodiments, the controller 106 is configured to adjust intensity of the UV light in response to a detected change in subject detection. For example, in response to determining at the controller 106, based on signals received from the sensors 108, that a subject was present in an area and then no longer present in that area, the controller 106 causes the intensity of the far-UVC light to be increased. In some embodiments, the subject detection sensors 108 are configured to determine the position of a subject and generate position data that is transmitted to the controller 106. Position data may include measurements or values representative of the position of a subject relative to the housing 102 and/or light source 104. In some embodiments, position data generated by the subject detection sensors 108 may indicate that no subject is detected by the sensors 108 and/or within range of the UV light emitted by the light source 106.

In some embodiments, the UV system 100 is configured to direct UV light toward areas where bacteria and/or pathogens are likely to be present. The subject detection sensors 108 may track the position of subjects within range of the far-UVC light over a period of time and transmit the subject's location and corresponding timestamps to the controller 106. Such areas may have a higher likelihood of containing pathogens or bacteria due to the presence of subjects. The controller 106 may be configured to store said data as historical position data and cause the light source 104 to emit far-UVC light targeted at areas in which subjects were detected. In some embodiments, the controller 106 is configured to cause the light source 104 to increase the intensity of far-UVC light emitted in those areas and/or direct the far-UVC light toward those areas. In some embodiments, the light source 104 is connected to one or more actuators such that the light source 104 may move relative to the housing 102 to direct the light emitted therefrom at a target location. In such embodiments, the controller 106 may be configured to control operation of the actuators to effect movement of the light source 104 relative to the housing 102 and focus the UV light emitted therefrom to areas with a higher likelihood of pathogen or bacteria presence. In some embodiments, the light source 104 is comprised of a plurality of bulbs or nodes that each emit UV light to a plurality of different locations that define the range of the light source 104. The controller 106 may be configured to cause a subset of said plurality to emit UV light at a greater intensity than the remainder of thereof such that UV light of a greater intensity may be focused in areas with a higher likelihood of pathogen or bacteria presence.

In some embodiments, the UV system 100 may be in communication with one or more client devices 110 external to the UV system 100. For example, the controller 106 may include a network interface device to allow the controller to be in communication with a client device 110 over a local area network (LAN), wireless area network (WAN), Bluetooth™, or any other communication means. The client devices 110 may be any computing device such as, but not limited to, a smart phone, a tablet computer, a laptop computer, and a desktop computer.

In one embodiment, the UV system 100 includes one or more computing devices (e.g., controller 106) having one or more processors and memory (e.g., one or more nonvolatile storage devices). In some embodiments, memory or computer readable storage medium of memory stores programs, modules and data structures, or a subset thereof for a processor to control and run the various systems and methods disclosed herein. In one embodiment, a non-transitory computer readable storage medium having stored thereon computer-executable instructions which, when executed by a processor, perform one or more of the methods disclosed herein.

The light source 104 may be configured to emit a UV light to clean and/or effect disinfection of the air and/or surfaces within the emission range of the emitted UV light. In some embodiments, when the UV system 100 includes more than one light source 104 for emitting UV light, the light sources 104 may output UV light at different intensities. In some embodiments, the light source 104 may be considered a UV germicidal irradiation (UVGI) light source. For example, the light source 104 may emit a UV light to kill or inactivate pathogens within the air and/or present on surfaces within the range of the emitted UV light. In some embodiments, the light source emits a far-UVC light. In some embodiments, the light source 104 is configured to emit a UV light having an output wavelength of between about 200 nanometers to about 254 nanometers. In some embodiments, the light source 104 is configured to emit a UV light having an output wavelength of between about 206 nanometers to about 230 nanometers. In some embodiments, the light source 104 is configured to emit a UV light having an output wavelength of about 222 nanometers.

In some embodiments, pathogens exposed to the far-UVC light emitted by light source 104 may be inactivated and inhaled by a subject, which may trigger an immune response. For example, pathogens that are weakened and/or inactivated due to exposure to the far-UVC light may be inhaled by a human subject in such a concentration as to trigger an immune response or provide a level of immunogenicity. The light source 104 may be configured to generate a far-UVC light that results in a sufficient volume of inactivated and/or live pathogens at a safe level, so as to provoke an immunogenic response in a subject.

The UV light emitted by the light source 104 may be safe for a subject to be exposed to (e.g., within the emission range of the emitted UV light) for an extended period of time. An extended period of time may be relative to conventional UVGI devices. For example, conventional UVGI devices emit a UV light having a significantly lower TLV value than the light source 104 of the present disclosure. Further to this example, conventional UVGI devices have a TLV value typically between 3 mJ/cm² to about 6 mJ/cm² whereas the light source 104 of the present disclosure may have a TLV value of about 23 mJ/cm². As such a subject may be exposed to the light emitted by the light source 104 for a period of time that is between about two to about eight times longer than the UV light emitted by a conventional UVGI device. In this manner, the light source 104 may be actively emitting UV light for an amount of time to effect disinfection of the air and/or surfaces while a subject (e.g., human being, domesticated animal, farm animal) is simultaneously exposed to the UV light and before causing any adverse effects or damage to the subject (e.g., before the subject is exposed for a predetermined amount of exposure time).

There may be a maximum amount of time the subject may be exposed to the UV light, after which the subject may experience adverse effects or damage caused by the UV light. The maximum amount of time a subject may be exposed to the UV light before any adverse effects or damage is caused may generally be referred to herein as a subject exposure limit, or exposure limit for short. In some embodiments, the exposure limit is based on one or more regulations established by governing bodies, such as, but not limited to the Occupational Safety and Health Administration (OSHA), ACGIH, IES, and/or American National Standards Institute (ANSI). In some embodiments, an exposure limit is based on a one day, or 24-hour, cycle of exposure. As such, the UV system 100 may be configured to adjust the intensity of emitted UV light and/or cease emitting the UV light such that a subject is not exposed beyond a maximum limit. The one or more subject detection sensors 108 may be configured to detect and monitor the presence of subjects within at least the emission range of the UV light emitted by the light source 104. In some embodiments, the UV system 100 includes a plurality of subject detection sensors 108 each in communication with the controller 106. There may be one, two, three, four, five, six, seven, eight, nine, ten or more than ten subject detection sensors 108a-108n in communication with the controller 106. In some embodiments, the one or more subject detection sensors 108 includes at least one of an infrared sensor and a motion sensor. For example, as illustrated in FIG. 2, the one or more subject detection sensors 108 may include four motion sensors 108a-108d and two infrared sensors 108e-108f In some embodiments, the one or more subject detection sensors 108 may include a proximity sensor. For example, one or more of sensors 108a-108d may be a proximity sensor. In some embodiments, the subject detection sensors 108 may include a combination of infrared sensors, motion sensors, and/or proximity sensors.

In some embodiments, the one or more subject detection sensors 108 may be oriented such that a detection range of the one or more subject detection sensors 108 overlaps with the emission range of the light source 104. For example, and as illustrated in FIG. 2, the light source 104 may emit UV light through a front panel 112 of the housing 102. The front panel 112 may include a window 114 through which the light source 104 may emit the UV light. The window 114 may be comprised of a transparent or translucent material such that the UV light emitted by the 104 may pass therethrough. In some embodiments, the window 114 may act as a bandpass filter. In some embodiments, there may be one or more other light sources (e.g., LED lights) positioned behind the window and configured to activate and deactivate in conjunction with the light source 104. For example, when the light source 104 is activated and emitting UV light, the other light sources may also be activated such that a light visible to subjects can be seen through the window 114. In this manner, the UV system 100 may provide a visual indication to subjects (e.g., human beings) that light source 104 is actively emitting UV light.

The one or more subject detection sensors 108 may be coupled to the housing 102 and oriented such that detection signals emitted by the subject detection sensors 108 are generally in the same direction as the emitted UV light. In this manner, the one or more subject detection sensors 108 may be able to detect the presence of subjects, via the emitted detection signals, which are within the emission range of the emitted UV light. In some embodiments, the subject detection sensors are positioned below the light source 104 and oriented generally toward the front of the housing 102. For example, the housing 102 may include a sensor panel 116 that the one or more subject detection sensors 108 are coupled to. In some embodiments, at least a portion of one or more of the subject detection sensors 108 may be exposed at an exterior surface of the sensor panel 116. For example, the subject detection sensors 108e and 108f are shown as extending partially through the sensor panel 116. One or more other subject detection sensors 108 may be entirely enclosed within the housing 102 (e.g., not exposed at an exterior surface of the sensor panel 116). For example, subject detection sensors 108a-108d are entirely enclosed within the housing 102 and positioned behind the sensor panel 116. As such, the sensor panel 116 may be comprised of a material configured to permit detection signals emitted by one or more of the subject detection sensors 108 to pass therethrough. In this manner, the sensor panel 116 may obscure one or more of the subject detection sensors 108 from the sight of a subject, while not impeding the operation of the subject detection sensors 108. In some embodiments, the housing 102 may include a base 118 configured to mount the UV system 100 to a surface within a public or private space. For example, the base 118 may include mounting hardware (e.g., mounting brackets, screw holes, adhesives) to enable a user to mount the UV system 100 to a wall, ceiling, or any other desired surface within the user's home. In some embodiments, the base 118 is configured to be mounted to and/or removed from an external bracket to enable a user to easily couple and decouple the housing 102 to the external bracket. In some embodiments, the base 118 may be configured to allow a user to adjust the orientation and/or position of the housing 102 when the base 118 is connected to a surface. In some embodiments, the UV system 100 may be portable and the mounting base 118 may be configured to be placed on a generally flat surface (e.g., floor, tabletop, a shelf).

As such, the one or more subject detection sensors 108 may emit detection signals to detect and/or monitor the presence of and/or position of one or more subjects. The subject detection sensors 108 may generate detection data based on the emitted detection signals and transmit the detection data to the controller 106. In this manner, the controller 106 may, based on the received detection data, determine whether a subject is within range of the UV light emitted by the light source 104 and/or an amount of time a subject has been within range of the UV light. In some embodiments, the controller 106 is configured to selectively adjust the intensity of, activate and/or deactivate the light source 104 based on the position of the subject relative to the UV light emission range, the time the subject has spent within the emission range of the UV light, and/or the proximity of the subject relative to the UV system 100. The controller 106 may be configured to automatically cause the light source 104 to emit or cease emitting UV light and/or adjust the intensity of the emitted UV light based on 1) the location of one or more subjects relative to the UV system 100 and/or 2) an amount of time a subject has been exposed to the emitted UV light.

In some embodiments, the controller 106 is configured to selectively activate and/or deactivate the one or more subject detection sensors 108. For example, the controller 106 may transmit a sensor activation signal to the one or more subject detection sensors 108, individually or in combination, to cause the one or more subject detection sensors 108 to activate and thereby emit subject detection signals. In some embodiments, the controller 106 is configured to activate the subject detection sensors 108 at a predetermined detection interval. For example, the controller 106 may transmit a sensor activation signal to the one or more subject detection sensors 108 at a predetermined detection interval of, but not limited to, less than one second, one second, two seconds, five seconds, ten seconds, thirty seconds, or one minute.

In some embodiments, the UV system 100 is configured to automatically cease emitting UV light and/or adjust the intensity of the UV light in response to a subject entering within a predetermined distance of the UV system 100. For example, the one or more subject detection sensors 108 may transmit detection data to the controller 106. Based on the detection data, the controller 106 may determine that a subject is within the range of the emitted UV light and is within the predetermined distance from the UV system 100. In response to determining that the subject is within the UV light range and the predetermined distance, the controller 106 may transmit a deactivation signal to the light source 104 to cause the light source 104 to cease emitting UV light. In some embodiments, the predetermined distance is a distance at which the UV light emitted by the light source 104 may cause adverse effects to or damage the subject. In some embodiments, the predetermined distance is about three feet. In some embodiments, one or more proximity sensors included in the subject detection sensors 108 may be used to determine whether a subject is within the predetermined distance. In some embodiments, the controller 106 is configured to adjust the intensity of the UV light based on the distance of a detected subject from the light source 104. For example, the controller 106 may be configured to decrease the intensity of the UV light as a detected subject moves closer to the light source 104.

In some embodiments, the UV system 100 is configured to distinguish between different subjects. For example, the UV system 100 may be configured to distinguish between a first subject and a second subject such that the UV system 100 may actively monitor the amount of time each subject has been exposed to UV light emitted by the light source 104. In some embodiments, the controller 106 is configured to distinguish between different subjects via the detection data generated by the subject detection sensors 108. For example, the detection data may include for each detected subject temperature profile data, and/or physical characteristic data (e.g., height, hair color, skin color, facial features). In this manner, the controller 106 may be configured to distinguish between different subjects. In some embodiments, there is a storage device (e.g., non-volatile memory, NAND die) operatively coupled to the controller 106 and configured to store detection data. As such, the controller 106 may associate a particular subject with subject specific detection data and transmit it to the storage device for storage and later retrieval.

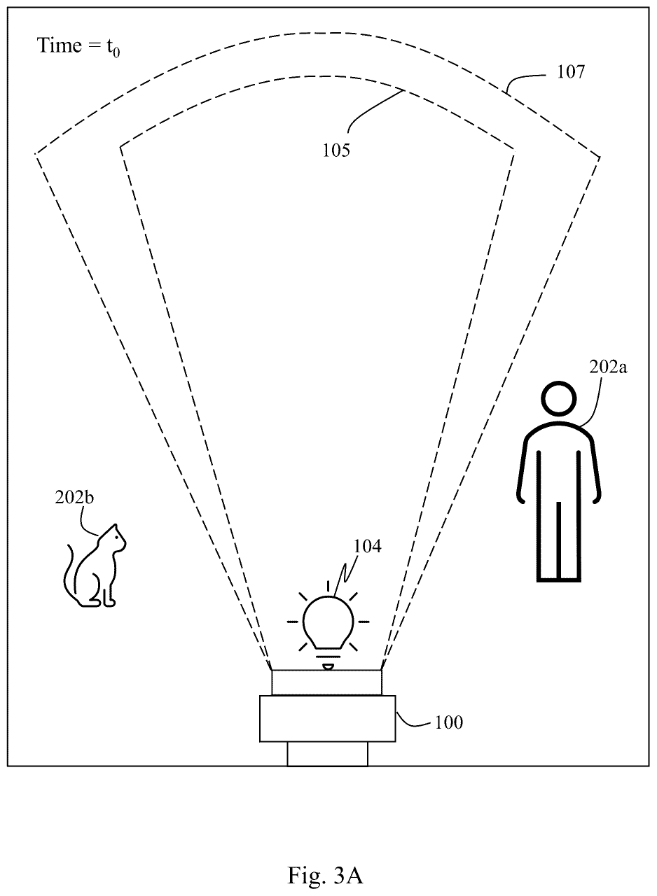

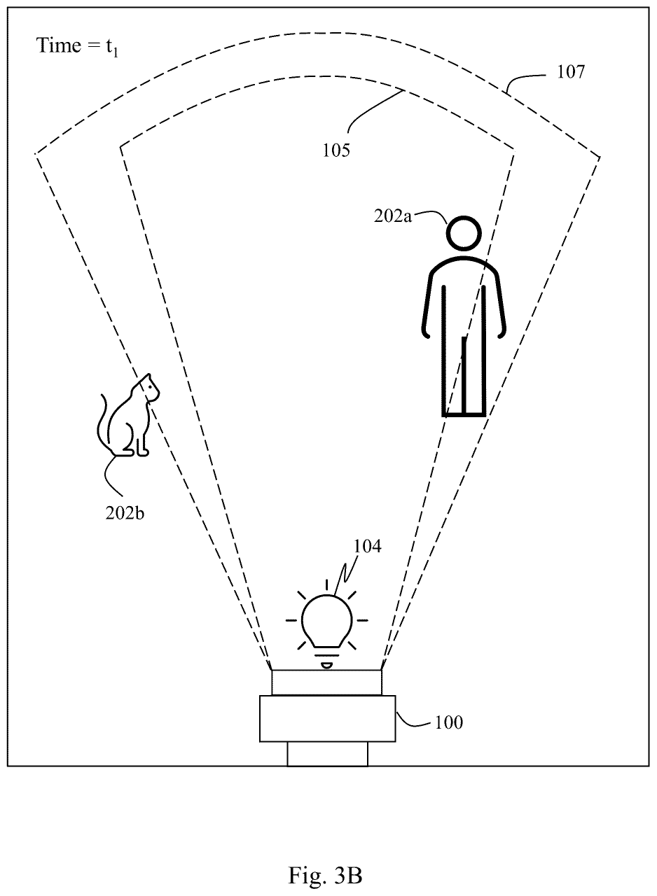

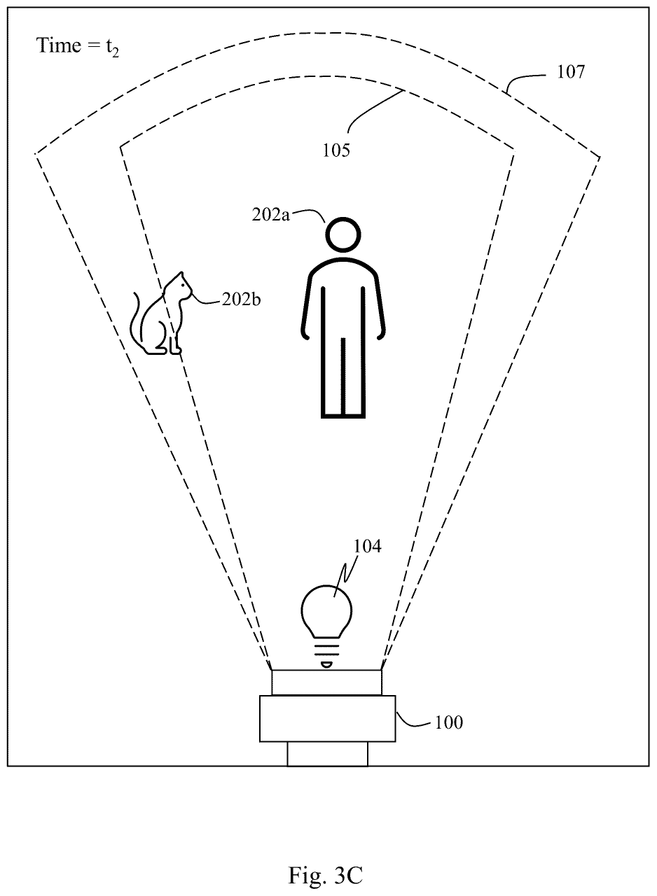

In some embodiments, the one or more sensors 108 includes an image capture device (e.g., camera, video recorder) to enable the controller 106 to distinguish between different subjects. As such, the image capture device may transmit to the controller 106 detection data including images of one or more subjects. The controller 106 may be configured to perform image recognition on the received detection data to determine physical characteristics of the one or more subjects and generate physical characteristic data for each subject. For example, the controller 106 may be configured to perform an optical recognition (e.g., facial recognition) based on the images included in the detection data to distinguish between different subjects. In some embodiments, the UV system 100 is configured to anonymize subjects whom facial recognition has been performed on to ensure that the subject's identity is kept secret. In some embodiments, the controller 106 is configured to leverage artificial intelligence (AI) and/or machine learning in order to distinguish between different subjects. In some embodiments, the controller 106 may be configured to distinguish between different subjects based on the subjects, height, weight, location, gait, and/or any other physical attributes. Referring to FIGS. 3A-7D, there are shown various use case diagrams illustrating one or more subjects' interaction with the UV system 100. The use case diagrams shown in FIGS. 3A-7D are intended to better illustrate aspects of the present disclosure, specifically in relation to the automatic activation and deactivation of the light source 104 by the controller 106 in response to detection data received from the one or more subject detection sensors 108. As discussed above, the controller 106 may include one or more processors configured to implement the functionality of the UV device described herein. For example, the controller 106 may have stored in a non-transitory computer readable storage medium, computer readable executable code that when executed by the processor implements the functionalities described herein.

In FIGS. 3A-7D, the emission range 105 of the UV light emitted by the light source 104 and the detection range 107 of the one or more subject detection sensors 108 are illustrated as broken lines. It will be understood though that the emission range 105 and detection range 107 shown are for purposes of illustrating concepts of the present disclosure and are not intended to limit the emission range 105 of the light source 104 and the detection range 107 of the subject detection sensors 108. For example, in some embodiments, the emission range 105 and detection range 107 may be generally equal such that each overlaps with one another. In some embodiments, the detection range 107 of the subject detection sensors 108 is greater than the emission range 105 of the light source 104, as illustrated in FIGS. 3A-7D. In FIGS. 3A-7D, the UV system 100 is mounted to a wall within a public or private space (e.g., a wall in a user's home).

Referring to FIGS. 3A-3C, there is illustrated a first use case of one or more subjects 202a-202b interacting with the UV system 100. In FIG. 3A, neither of the subjects 202a-202b is within the emission range 105 of the light source 104 or the detection range 107 of the subject detection sensors 108 at time t₀. At time t₀, the controller 106 may receive detection data from the one or more subject detection sensors 108. The controller 106, may determine, based on the received detection data, that no subject is within the emission range 105 of the UV light emitted by the light source 104. In response to determining that no subject is within the emission range 105, the controller 106 may cause the light source 104 to emit the UV light.

In some embodiments, the controller 106 is configured to cause the intensity of emitted UV light to increase in response to determining that no subject is within the emission range 105. For example, the controller 106 may be configured to determine whether a subject moves outside of the emission range 105 based on detection data received from the sensors 108 and automatically increase the intensity of the UV light emitted by the light source 104.

In FIG. 3B, at a time t₁ occurring after the time t₀, the first subject 202a has moved within the emission range 105 and the second subject 202b has moved within the detection range 107. The controller 106 may be configured to determine, based on the detection data received from the subject detection sensors, that a subject is within range of the UV light emitted by the light source 104. For example, at time t₁ the subject detection sensors 108 may transmit detection data to the controller 106. The controller 106 may determine, based on the received detection data, that the first subject 202a is within the emission range 105 and that the second subject 202b is not. In FIG. 3B, time t₁ corresponds to the point in time at which the first subject 202a has moved from outside the emission range 105 to inside the emission range 105. The controller 106 may be configured to determine, based on the detection data, the amount of time a subject has been exposed to the emitted UV light. For example, the controller 106 may determine at time t₁ that the first subject 202a has been exposed to the emitted UV light for about one second and that the second subject 202b has not been exposed to the emitted UV light.

In some embodiments, the controller 106 is configured to cause the light source 104 to cease emitting the UV light based on the determined amount of time. For example, the controller 106 may be configured to determine if a subject has been within the emission range 105 for a predetermined amount of exposure time and if so, cause the light source 104 to cease emitting the UV light. The predetermined amount of exposure time may correspond to the subject exposure limit amount of time. In some embodiments, the predetermined amount of exposure time may be less than or equal to the exposure limit for a subject being exposed to the UV light emitted by the light source 104. In this manner, the controller 106 may cause the light source 104 to cease emitting the UV light prior to a subject being exposed thereto for the exposure limit amount of time. For example, if the subject exposure limit is fifteen minutes, then the predetermined amount of exposure time may be less than or equal to fifteen minutes. In some embodiments, the predetermined amount of exposure time is between about 20% to about 80% of the subject exposure limit amount of time. For example, if the subject exposure limit amount of time is fifteen minutes, the predetermined amount of exposure time may be six minutes.

In some embodiments, the controller 106 is configured to decrease the intensity of the UV light emitted by the light source 104 based on the determined amount of time. The controller 106 may be configured to calculate the UV dose a subject has received based, at least partially on the intensity of the emitted UV light and the determined amount of time. In response to the determined UV dose being within a predetermined range of the TLV, the controller 106 may be configured to cause the intensity of the emitted UV light to be decreased and/or ceased to be emitted entirely. For example, if a determined UV dose is within 80-90% of the TLV the controller 106 may cause the intensity of the emitted UV light to be decreased.

The controller 106, in response to determining that the first subject 202a has not been exposed to the emitted UV light for a predetermined amount of exposure time, may cause the light source 104 to continue emitting the UV light. For example, the light source 104 was activated in FIG. 3A at time t₀ and at time t₁ the controller 106 determines that the first subject 202a has not been exposed to the UV light for the predetermined amount of exposure time. As such, the controller 106 may not transmit a deactivation signal to the light source 104, thereby causing the light source 104 to continue emitting UV light.

In FIG. 3C, at a time t₂ occurring after the time t₁ the first subject 202a has remained within the emission range 105 and the second subject has moved within the emission range 105. In some embodiments, the controller 106 is configured to, in response to determining that a subject is within the emission range 105 for the predetermined amount of exposure time, cause the light source 104 to cease emitting the UV light. The controller 106 may receive between times t₁ and t₂ detection data from the subject detection sensors 108. For example, the controller 106 may cause the subject detection sensors 108 to emit a detection signal at a predetermined detection interval (e.g., every second) between times t₁ and t₂. Each time the subject detection sensors 108 are activated, detection data may be generated by the subject detection sensors 108 and transmitted to the controller 106.

The controller 106 may determine, based on the received detection data over a period of time (e.g., the period of time between times t₁ and t₂) the amount of time a subject has been within the emission range 105. For example, the controller 106 may determine that there has been a subject (e.g., first subject 202a, second subject 202b) within the emission range 105 for the period of time between times t₁ and t₂ based on the detection data received between those times. In response to the amount of time a subject has been within the emission range being greater than or equal to the predetermined amount of exposure time, the controller 106 may cause the light source 104 to cease emitting the UV light. For example, in FIG. 3C, the first subject 202a has been within the emission range 105 for an amount of time equal to t₂−t₁. The amount of time t₂−t₁ determined by the controller 106 may be equal to the predetermined amount of exposure time (e.g., six minutes), and as such, the controller 106 may transmit a deactivation signal to the light source 104 to cause the light source 104 to cease emitting UV light. In some embodiments, the controller 106 is configured to cause the light source 104 to cease emitting UV light in response to the one or more subject detection sensors 108 detecting the presence of a subject within the range of the emitted UV light for between about one minute to about ten minutes. Put another way, the predetermined amount of exposure time may be between about one minute to about ten minutes.

In some embodiments, the UV system 100 is configured to cause the light source 104 to cease emitting UV light in response to a subject moving within a predetermined distance of the UV system 100. For example, and referring back to FIG. 3B, if the first subject 202a had moved within a predetermined distance of the light source 104 at time t₁ and was within the emission range 105, the controller 106 may cause the light source 104 to cease emitting UV light at time t₁. In some embodiments, the controller 106 is configured to cease emitting UV light at the time that the subject detection sensors 108 determine that a subject is within the predetermined distance from the light source 104 regardless of the amount of time the subject has been exposed to or not exposed to the emitted UV light. For example, at time t₁ the first subject 202a was first detected as being within the emission range 105. Regardless of an amount of time the first subject 202a had been exposed to the emitted UV light, the controller 106 may, in response to receiving detection data from the subject detection sensors 108 (e.g., one or more proximity sensors), determine that the first subject 202a is within the predetermined distance from the light source 104 and transmit a deactivation signal to the light source 104. In some embodiments, the predetermined distance is between about one foot to about eight feet. In some embodiments, the predetermined distance is about three feet. In some embodiments, if a subject is outside of the predetermined distance, the controller 106 may control operation of the light source 104 based on the predetermined amount of exposure time as discussed above.

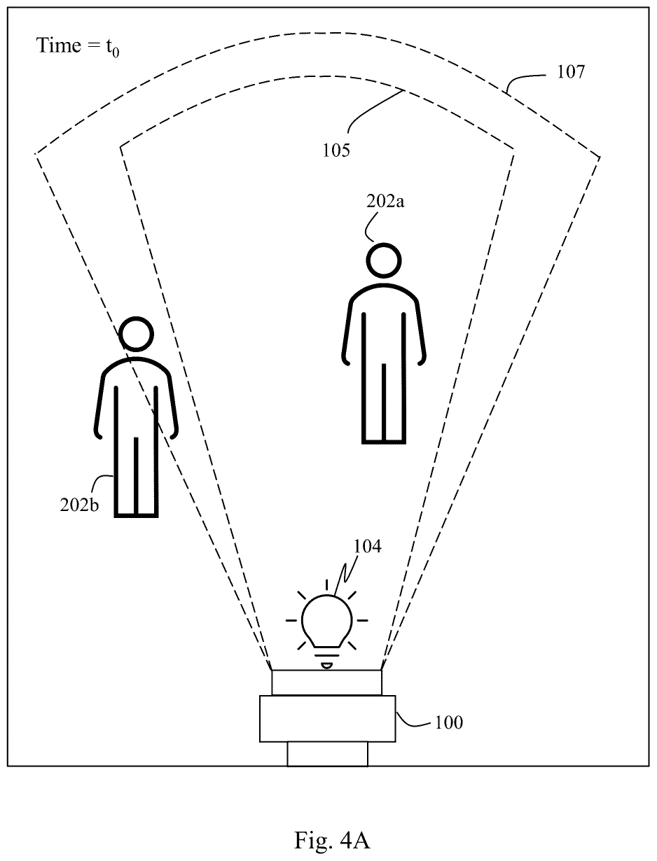

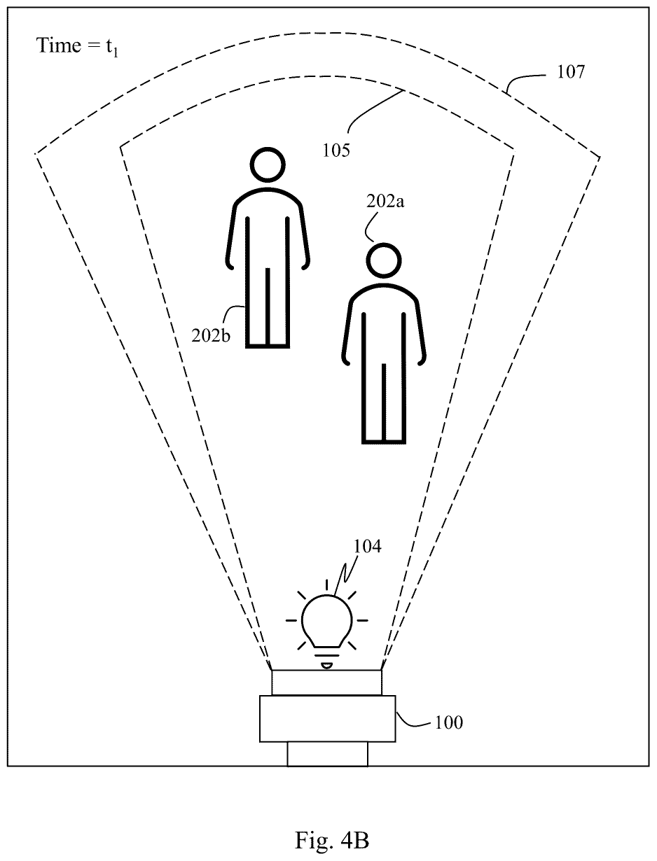

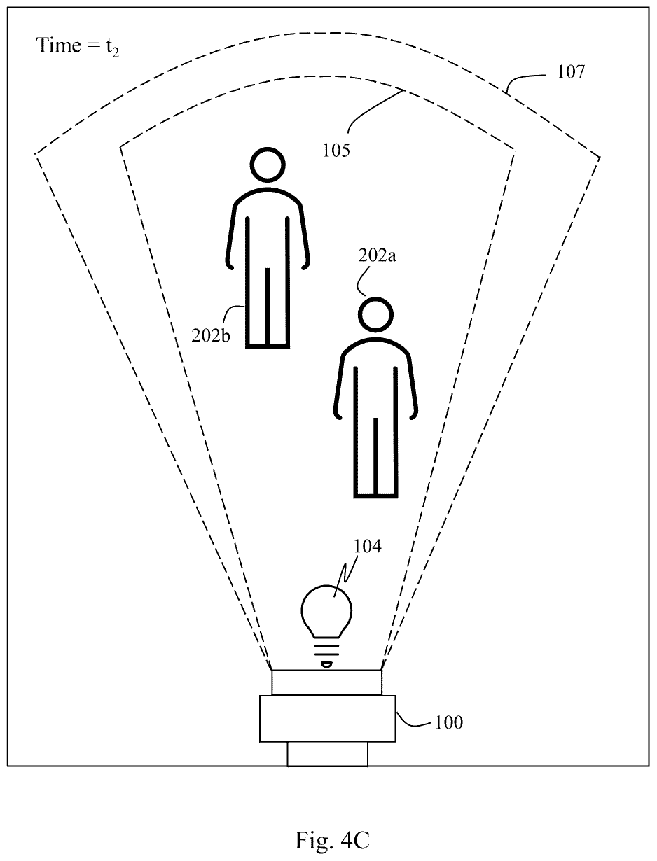

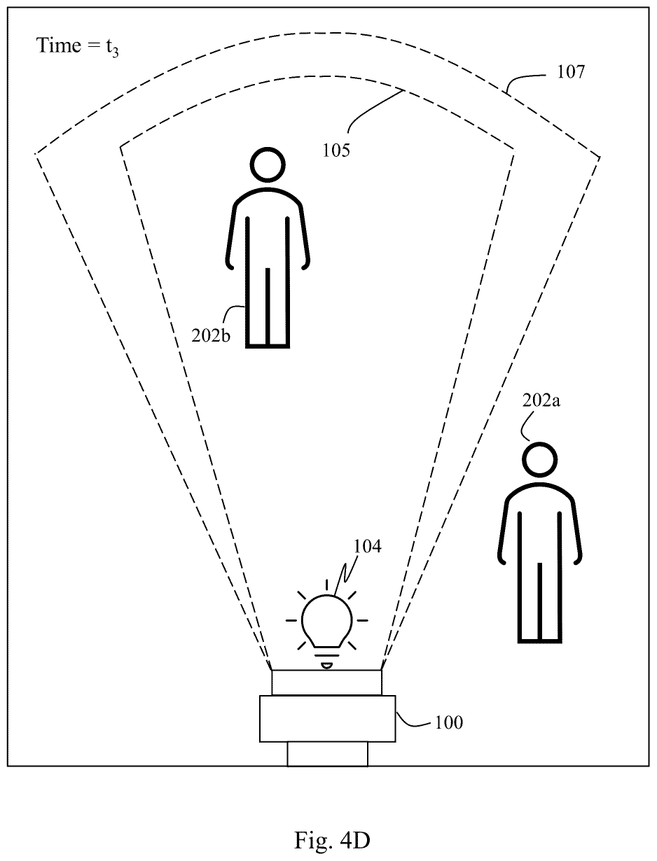

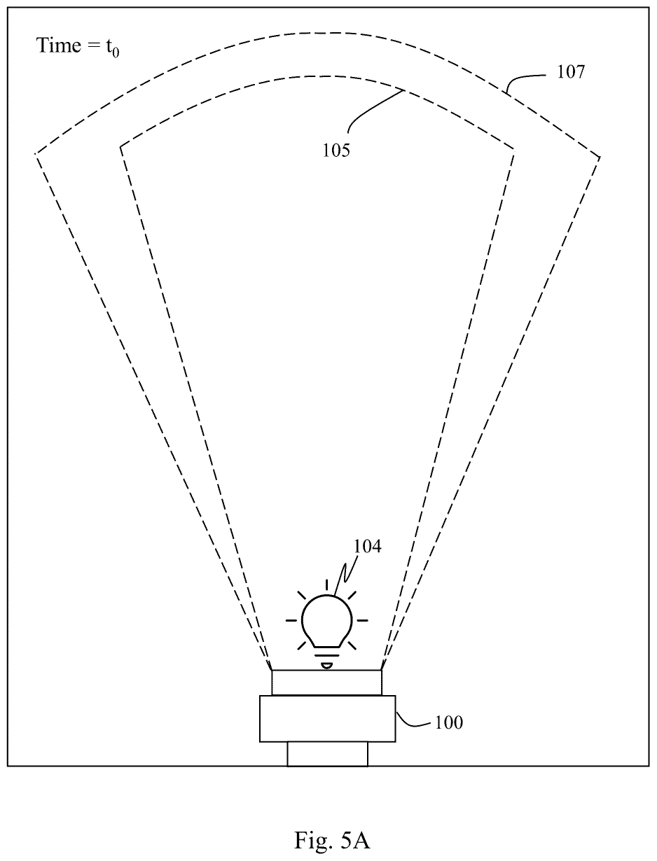

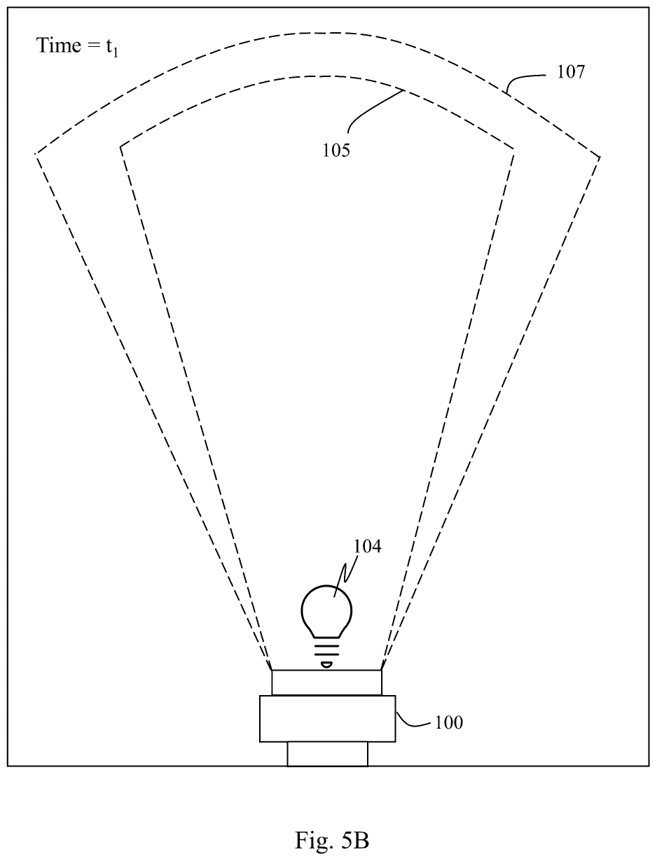

In some embodiments, the controller 106 is configured to determine an amount of time that a particular subject of one or more subjects has been exposed to the UV light emitted by the light source 104. Referring to FIGS. 4A-4D, there is shown a second use case in which the controller 106 is configured to distinguish between different subjects and determine an amount of exposure time for each subject. As discussed above, the controller 106 may be configured to distinguish between different subjects based on the detection data generated by the one or more subject detection sensors 108. For example, in FIG. 4A, a first subject 202a is located within the emission range 105 and a second subject 202b is located outside the emission range 105 but within the detection range 107. At time t₀ the subject detection sensors 108 may transmit detection data to the controller 106 and the controller 106 may determine that there is a first subject 202a within the emission range 105 of the light source 104 and that there is a second subject 202b outside the emission range 105. Put another way, based on the received detection data at time t₀ the controller 106 may distinguish between the first and second subject 202a-202b.

The controller 106 may determine, based on the detection data received at time t₀ that the first subject 202a has not been within the emission range 105 for a period of time greater than or equal to the predetermined amount of exposure time (e.g., six minutes). As such, the controller 106 may cause, or may have previously caused, the light source 104 to emit UV light. As such, in FIG. 4A the light source 104 is actively emitting UV light to effect disinfection within the emission range 105 while the first subject 202a is within the emission range 105.

In FIG. 4B, at time t₁ occurring after time t₀ the second subject 202b is located within the emission range 105 simultaneously with the first subject 202a. At time t₁ the subject detection sensors may generate and transmit detection data to the controller 106. Furthermore, in the period of time between time t₁ and time t₀ the subject detection sensors 108 may have generated and transmitted detection data to the controller 106. The controller 106 may be configured to determine, based on the received detection data between times t₁ and t₀, the amount of time the first subject 202a and the amount of time the second subject 202b have been within the emission range 105. For example, if the amount of time between t₁ and t₀ is three minutes then the controller 106 may determine, based on the received detection data, that the first subject 202a has been within the emission range 105 for three minutes and that the second subject 202b has been within the emission range for less than one second (e.g., the second subject 202b moved within the emission range 105 at time t₁). In this manner, the controller 106 may be configured to determine the amount of exposure time for each of the first subject 202a and second subject 202b.

The controller 106 may determine, based on the determined amount of exposure time for each of the subjects 202a, 202b, whether to cause the light source 104 to cease emitting the UV light. For example, if the predetermined amount of time is about six minutes and the controller 106 determines, based on the received detection data, that the first subject 202a has been exposed for three minutes and the second subject 202b has been exposed for less than one second, the controller 106 may not transmit a deactivation signal to the light source 104. As such, in FIG. 4B, the light source 104 at time t₁ continues to emit UV light.

In FIG. 4C, at a time t₂ occurring after the time t₁ the first subject 202a and second subject 202b remain located within of the emission range 105. The one or more subject detection sensors 108 may generate and transmit detection data to the controller 106 at the predetermined detection interval between times t₂ and t₁. The controller 106 may determine, based on the received detection data between times t₂ and t₁, the amount of time that each of the first subject 202a and second subject 202b have been within the emission range 105. The determined amount of exposure time for the first subject 202a may be generally equal to t₂−t₀. The amount of exposure time for the second subject 202b, determined by the controller 106, may be generally equal to t₂−t₁ where t₁ is the time at which the second subject 202b was first determined to be within the emission range. If the exposure time for the first subject 202a is equal to the predetermined amount of exposure time, but the amount of exposure time for the second subject is less than the predetermined amount of exposure time the controller 106 may transmit a deactivation signal to the light source 104 thereby causing the light source 104 to cease emitting UV light.

In FIG. 4D, at a time t₃ occurring after the time t₂, the first subject 202a has moved outside of the emission range 105. The subject detection sensors 108 may generate and transmit detection data to the controller at the predetermined detection interval between times t₃ and t₂. The controller 106 may determine, based on the received detection data, that at time t₃ the second subject 202b is within the emission range 105 and that the first subject 202a is not. The light source 104 between the times t₃ and t₂ was not active and as such was not emitting UV light. Therefore, between the times t₃ to t₂ neither of the first subject 202a and second subject 202b were exposed to UV light from the light source 104. As such, at time t₃ the controller 106 may determine the amount of exposure time for the second subject 202b. In this instance, the determined amount of exposure time for the second subject 202b is equal to t₂−t₁.

The controller 106 may determine whether the amount of exposure time for the second subject 202b is less than the predetermined amount of exposure time. For example, the amount of exposure time t₂−t₁ determined by the controller 106 for the second subject 202b may be three minutes and the predetermined amount of exposure time may be six minutes. As such, the controller 106, in this instance, may determine that the second subject 202b has not been exposed to UV light emitted by the light source 104 for the predetermined amount of time. The controller 106 may, in response to determining that the second subject 202b has not been exposed to the UV light for the predetermined amount of time, transmit an activation signal to the light source 104 to cause the light source 104 to emit UV light. In this manner, the UV system 100 may distinguish between different subjects in order to optimize an amount of time the UV system 100 may safely emit UV light to effect disinfection of the air and/or surfaces within the emission range 105.

In other embodiments, the controller 106 may be configured to activate and/or deactivate the light source 104 based on an aggregate amount of time one or more subjects have been exposed to the emitted UV light. For example, in FIG. 4D, the controller 106 determined that the second subject 202b was the only subject within the emission range 105 and that the second subject 202b had not been exposed to emitted UV light for the predetermined amount of exposure time and therefore causes the light source 104 to be activated. However, alternative to what is illustrated in FIG. 4D, in instances where the controller 106 is configured to control activation of the light source 104 based on an aggregation of determined subject exposure time, the controller 106 at time t₃ may not cause the light source 104 to activate. For example, the controller 106 at time t₃ may determine that the total amount of time that any subject 202a-202b has been within the emission range 105 is generally equal to t₃−t₀ which is greater than the predetermined amount of exposure time. As such, at time t₃ the controller 106 may not transmit an activation signal to the light source 104, because the light source 104 would have already been deactivated at time t₂ when the first subject 202a was exposed to the UV light for the predetermined amount of exposure time.

In some embodiments, the UV system 100 may be configured to cease emitting UV light in response to UV light being continuously emitted for a predetermined maximum period of time. Referring to FIGS. 5A-5B, there is illustrated a third use case in which the UV device emits UV light for up to a predetermined maximum period of time. In FIG. 5A at an initial time t₀ the light source 104 is activated and thereby is emitting UV light. The activation of the light source 104 may be in response to the controller 106 determining that no subjects are within the emission range 105 or that a subject within the emission range 105 has not remained therein for the predetermined amount of exposure time. For sake of brevity, it will be assumed in FIG. 5A that the controller 106 determines based on detection data generated by the subject detection sensors 108 at time t₀ that no subjects are within the emission range 105 and causes the light source 104 to emit UV light.

In FIG. 5B, at time t₁ occurring an amount of time after time t₀ the UV system 100 is still emitting UV light. The controller 106 may be configured to determine the amount of time that the light source 104 has been continuously emitting UV light. For example, the controller 106 may be in communication with the light source 104 such that the controller 106 may determine whether the light source 104 is active or not. In this manner, the controller 106 may determine that the light source 104 has been continuously emitting UV light for an amount of time equal to t₁−t₀. In response to the controller 106 determining at time t₁ that the amount of time t₁−t₀ is equal to a predetermined maximum emission amount of time, the controller 106 may cause the light source 104 to cease emitting UV light.

In some embodiments, the predetermined maximum emission amount of time may be less than or equal to the predetermined exposure limit for subjects exposed to the emitted UV light. In this manner, the UV system 100 may automatically cease to emit UV light prior to an exposure limit of a subject being reached. As such, the controller 106 being configured to deactivate the light source 104 at the predetermined maximum emission amount of time may act as an automatic safety measure to ensure no subject is exposed to UV light for a period of time great enough to cause adverse effects or damage thereto. In some embodiments, the predetermined maximum emission amount of time may be between about six minutes to about sixty minutes. In some embodiments, the predetermined maximum amount of time may be about ten minutes, about fifteen minutes, about twenty minutes, about twenty-five minutes, about thirty minutes, about thirty-five minutes, about forty minutes, about forty-five minutes, about fifty minutes, about fifty-five minutes, or about sixty minutes. In some embodiments, the predetermined maximum amount of time may be about two hours, three hours, four hours, five hours, six hours, seven hours, eight hours, nine hours, ten hours, eleven hours, twelve hours, thirteen hours, fourteen hours, fifteen hours, sixteen hours, seventeen hours, eighteen hours, nineteen hours, twenty hours, twenty-one hours, twenty-two hours, twenty-three hours, or twenty-four hours, within a twenty-four hour time cycle. In some embodiments, the predetermined maximum emission amount of time may be about fifteen minutes.

In some embodiments, the UV system 100 may be configured to calculate an effective disinfection rate based on an amount of time that the light source 104 has been emitting the UV light. For example, the controller 106 may monitor the amount of time which the light source 104 has been emitting UV light. The controller 106 may determine, based on the monitored amount of time, an effective disinfection rate representing the efficacy of disinfection of the air and/or surfaces within the range of the emitted UV light. In some embodiments, the controller 106 may transmit the determined effective disinfection rate to the client device 110. In this manner, a user of the client device 110 may be provided with a visual indication of the efficacy of the disinfection effected by the UV system 100.

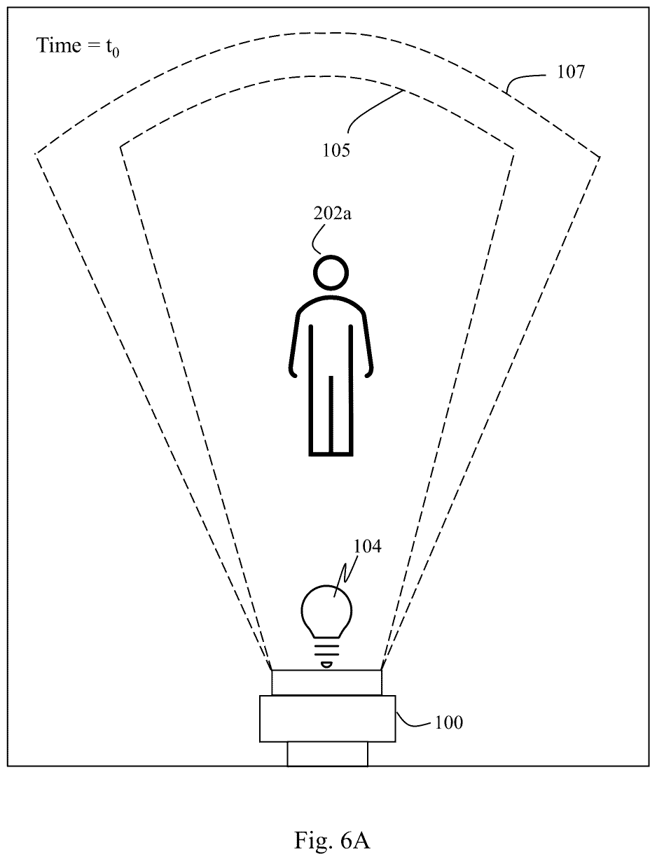

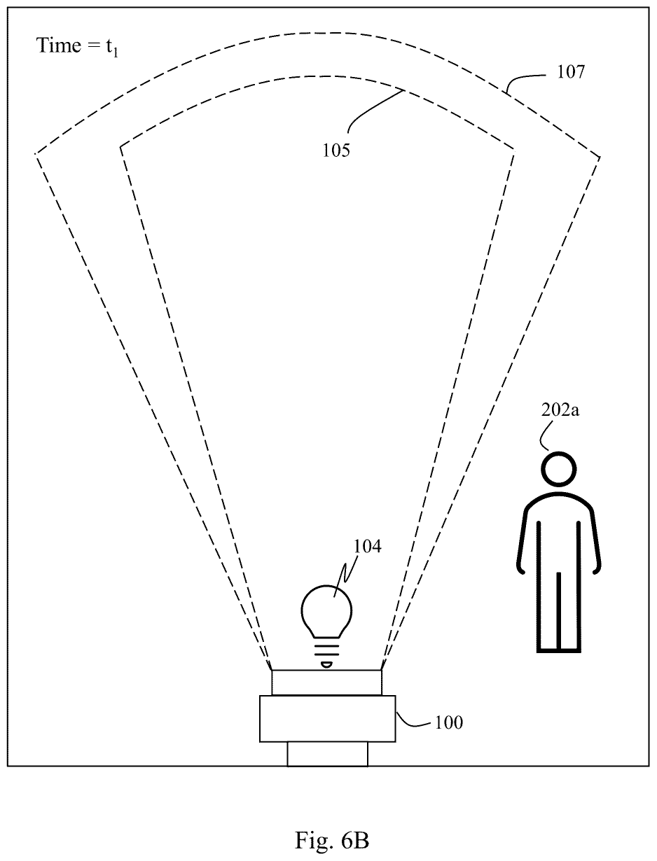

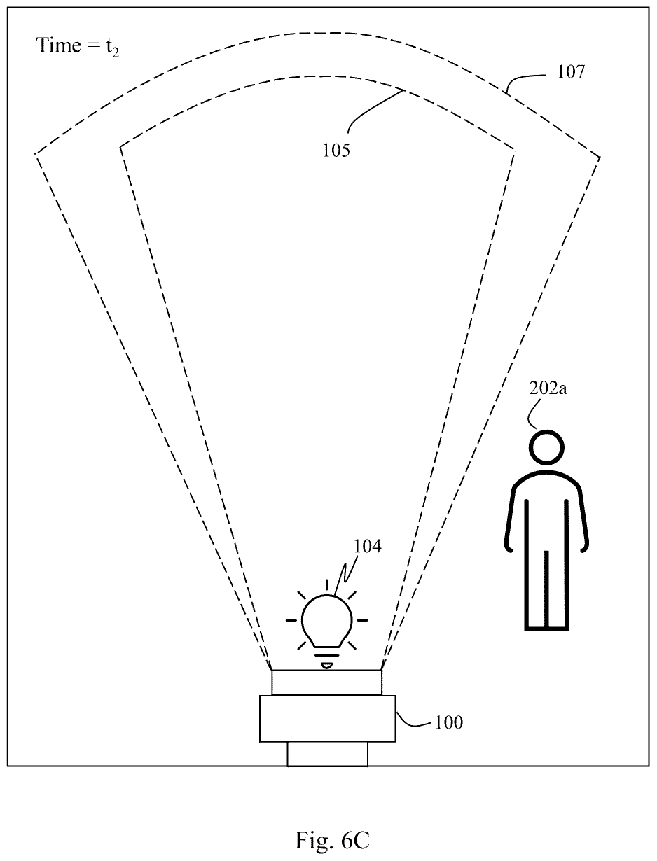

In some embodiments, the UV system 100 is configured to delay reactivation of the light source 104 by a predetermined amount of delay time. Referring to FIGS. 6A-6C, the light source 104 may be deactivated by the controller 106 in response to a subject being within range of the emitted UV light for the predetermined amount of exposure time (e.g., as discussed above with reference to FIGS. 3A-3C) or the UV light source emitting UV light for the predetermined maximum emission amount of time (e.g., as discussed above with reference to FIGS. 5A-5B). In FIG. 6A, the light source 104 is deactivated at time t₀ in response to the subject 202a being within the emission range 105 for the predetermined amount of exposure time. The controller 106 may be configured to begin tracking the amount of time that the light source 104 has not been active. For example, at time t₀ the controller 106 may begin recording the amount of time that the light source has not been active.

In FIG. 6B, at time t₁ occurring an amount of time after time t₀ the subject 202a has moved outside of the emission range 105. The controller 106 may determine that the subject 202a has moved outside of the emission range 105 and determine whether the amount of time between times t₁ and t₀ is greater than or equal to the predetermined amount of delay time. In FIG. 6B, the controller 106 determines that the amount of time t₁−t₀ is not greater than or equal to the predetermined amount of delay time and therefore does not cause the light source 104 to emit UV light. As such, the light source 104 has not been active between times t₁ and t₀.

In FIG. 6C, at time t₂ occurring an amount of time after time t₁ the subject 202a has remained outside of the emission range 105. The controller 106 may determine that no subject is within the emission range 105 and may determine whether the amount of time elapsed between times t₂ and t₀ are greater than or equal to the predetermined amount of delay time. In response to the controller 106 determining that the amount of time t₂−t₀ is greater than or equal to the predetermined amount of delay time, the controller 106 may cause the light source 104 to emit UV light. In some embodiments, the controller 106 is configured to cause the light source 104 to emit UV light in response to the light source 104 being deactivated for the predetermined amount of delay time regardless of whether a subject is within the emission range 105. In other embodiments, the controller 106 may be configured to only activate the light source 104 in response to determining that no subject is within the emission range and that the predetermined amount of delay time has been reached. In some embodiments, the predetermined amount of delay time is between about one second to fifteen minutes. In some embodiments, the predetermined amount of delay time is about six minutes.

In some embodiments, the UV system 100 may be configured to emit UV light based on one or more user inputs transmitted from the client device 110 to the UV system 100. For example, a user may, at the client device 110 input one or more desired activation times corresponding to one or more periods of time the user wishes the UV system 100 to emit UV disinfecting light. Put another way, the user may, at the client device 110 input a desired schedule with which the user wishes the UV device to operate within. In some embodiments, the UV system 100 is configured prioritize safety operations (e.g., emission and ceasing emission of UV light based on the safety operations described above, for example, with reference to FIGS. 3A-6C) over the user input desired schedule. As such, the schedule input by the user may include an indication of one or more start times and stop times corresponding to when the UV system 100 should be emitting UV light and when the UV system 100 should not.

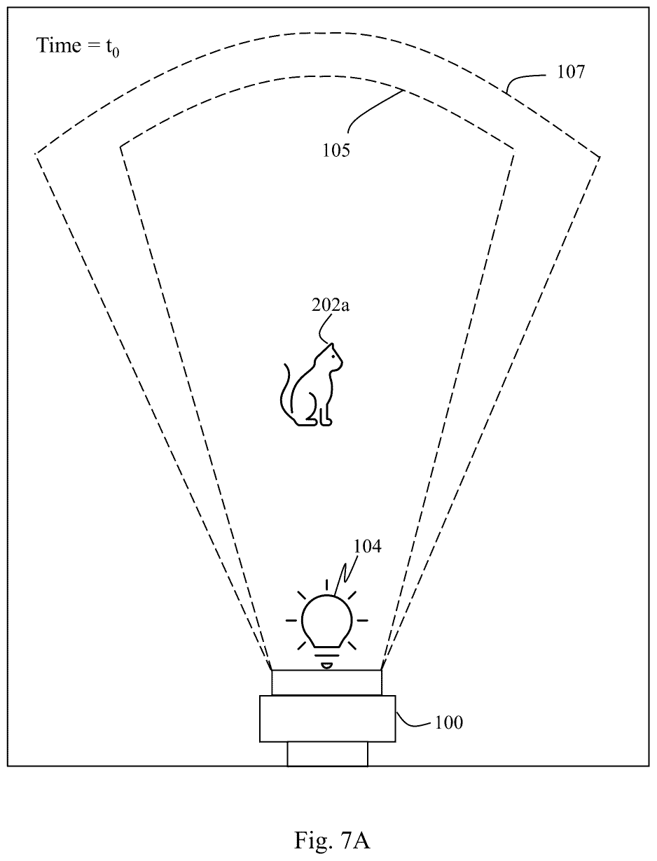

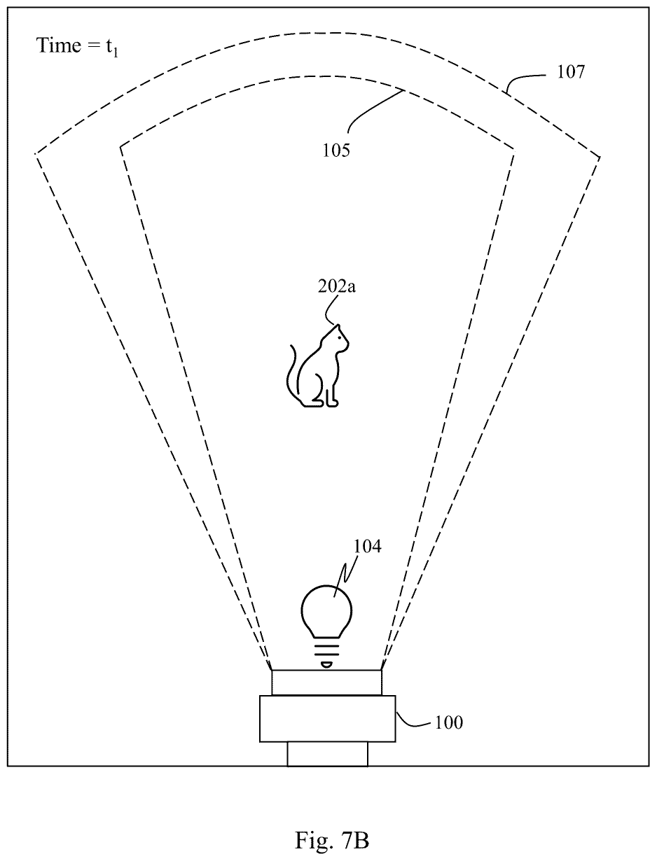

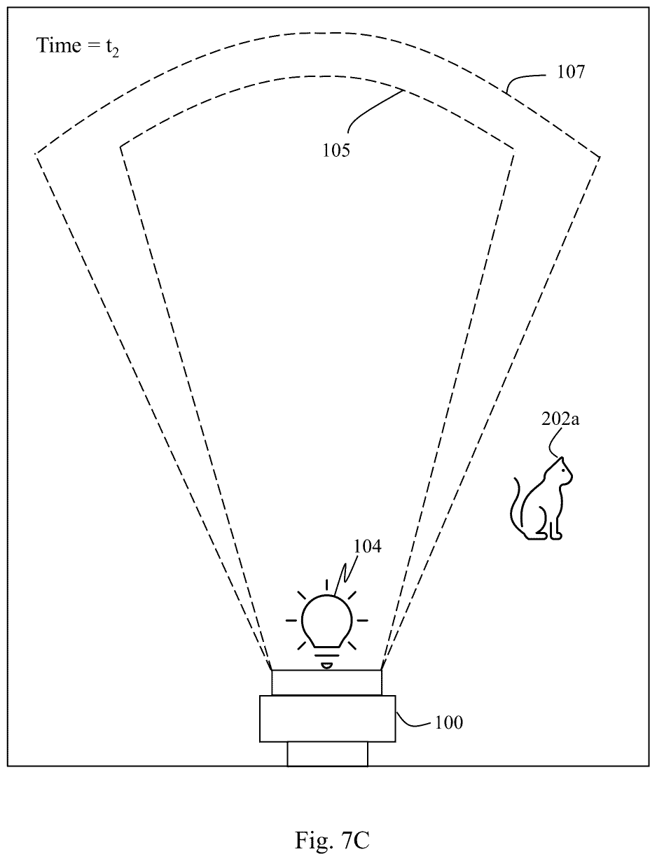

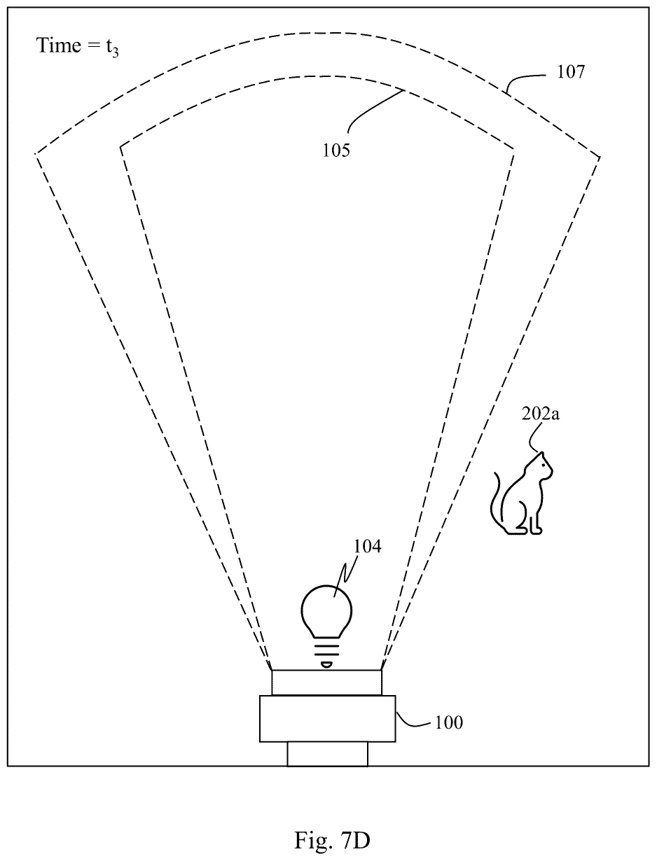

Referring to FIGS. 7A-7D, there is shown a use case illustrating the UV system 100 operating within a desired continuous activation period input by a user at the client device 110. Prior to what is illustrated in FIGS. 7A-7D, the user may input, at the client device 110, an indication of a desired continuous activation period, or, put another way an indication of a continuous period of time within which the user wishes for the UV system 100 to emit UV light. The indication of a desired continuous activation period may be defined by a start time and a stop time. For example, the desired continuous activation period may be defined by a start time of 10:00 AM and a stop time of 4:00 PM. In this manner, the UV system 100 may enable a user to remotely control operation of the UV system 100. This may be beneficial such that the user may define different operating times of the UV system 100 corresponding to times in which the user may be away from the UV device (e.g., when the user is at work, school, or generally out of their home or a room in which the UV system 100 is located). The indication of the continuous activation period may be transmitted from the client device 110 to the controller 106.

In FIG. 7A, at time t₀ the controller 106 determines that the time t₀ corresponds to the start time of the desired continuous activation period. As such, at time t₀ the controller 106 may transmit the activation signal to the light source 104 to cause the light source 104 to emit UV light. In some embodiments, the controller 106 may be configured to activate the light source 104 in response to the current time (e.g., time t₀) being the same as the desired start time and in response to determining that a subject within the emission range 105 has not been exposed to UV light within the emission range 105 for the predetermined amount of exposure time.

For example, in FIG. 7A, a subject 202a (e.g., a domesticated animal) is within the emission range 105. The controller 106 may have, prior to the time t₀ been receiving detection data from the subject detection sensors and determined that the subject 202a was within the emission range 105. In one instance the light source 104 may be active prior to time t₀ and the subject 202a may be within the emission range 105 for a period of time less than the predetermined amount of exposure time. As such, in response to the time t₀ coinciding with the start time, the controller 106 may transmit the activation signal to the light source 104 at time t₀. In another instance, the light source 104 may have been active prior to time t₀ and the subject 202a may have been within the emission range 105 for a period of time equal to the predetermined amount of exposure time. As such, at time prior to time t₀ the controller 106 may cause the light source 104 to cease emitting UV light. Furthermore, in an instance where the subject 202a remains within the emission range 105 at time t₀ the controller 106 may not cause the light source 104 to emit UV light even though the time t₀ coincides with the user input start time.

For sake of brevity though, it will be assumed at time t₀ in FIG. 7A that the light source 104 is activated. In FIG. 7B, at a first time t₁ following the start time t₀ and prior to the user specified end time, the controller 106 may determine that one or more subjects are within the emission range 105 of the activated light source 104. In some embodiments, in response to the controller 106 determining that a subject (e.g., subject 202a) being within the emission range 105 at time t₁ the controller 106 may transmit a deactivation signal to the light source 104 to cause the light source 104 to cease emitting UV light. In other embodiments, the controller 106 may determine, based on detection data received at the predetermined detection interval between times t₀ and t₁ that the subject 202a has remained within the emission range 105 for the predetermined amount of exposure time. As such, the controller 106 at time t₁ may transmit the deactivation signal to the light source 104.

In FIG. 7C, at a second time t₂ following the first time t₁ and prior to the user specified stop time, the controller 106 may determine that no subject is within the emission range 105 of the light source 104 and may cause the light source 104 to emit UV light. For example, in FIG. 7C, at time t₂ the subject 202a has moved outside of the emission range 105. The controller 106 determines, based on received detection data received from the subject detection sensors 108 at time t₂ that no subject is within the emission range 105. In response to determining that no subject is within the emission range 105 and that the time t₂ falls within the desired continuous activation period, the controller 106 transmits the activation signal to the light source 104 to cause the light source 104 to emit UV light.

In FIG. 7D, at a third time t₃, the controller 106 may determine that the third time t₃ coincides with, or is equal to, the user specified end time. In response to determining that the third time t₃ is equal to the user specified end time, the controller 106 may transmit a deactivation signal to the light source 104 to cause the light source 104 to cease emitting UV light. In some embodiments, in response to determining that the third time t₃ is equal to the end time, the controller 106 may cause the light source 104 to cease emitting UV light regardless of whether a subject is within the emission range 105 or not. For example, in FIG. 7D, there are no subjects within the emission range 105, however, the controller 106 determines that the user specified end time has been reached and therefore causes the light source 104 to cease emitting UV light.

In some embodiments, the UV system 100 may be configured to perform redundant detection for subjects within the emission range of the light source 104 to improve the safety of the UV system 100. For example, aspects of the present disclosure are discussed above in FIGS. 3A-7D with reference to the generation of detection data by one or more subject detection sensors 108 and determining, at the controller 106, a position of one or more subjects relative to the emission range 105. In this manner, the controller 106, or a processor included therein, and one or more of the subject detection sensors 108 may act as a first safeguard system for ensuring that subjects are not exposed to UV light emitted by the light source 104 for an amount of time that would cause adverse effects and/or damage. As such, the UV system 100 may include a second safeguard system, which may include additional subject detection sensors (not shown) and/or a subset of the subject detection sensors 108 illustrated in FIGS. 1-2. Furthermore, the second safeguard system may include a second processor and/or second controller generally the same as controller 106 or a processor included therewith. The second safeguard system may be configured to operate in generally the same manner as, and independently of, the first safeguard system and as such will not be discussed in extensive detail for sake of brevity. However, it should be understood that the UV system 100 may be configured to leverage both the first and second safeguard systems simultaneously to automatically activate or deactivate the light source 104 as described above.

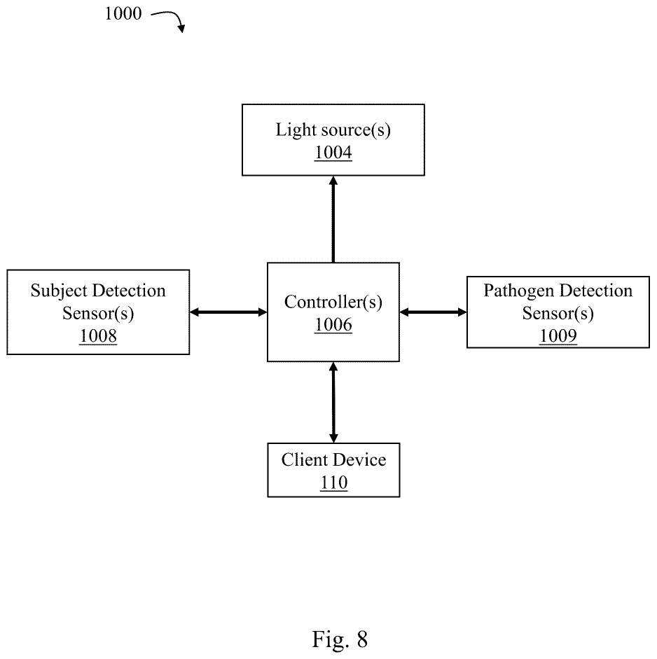

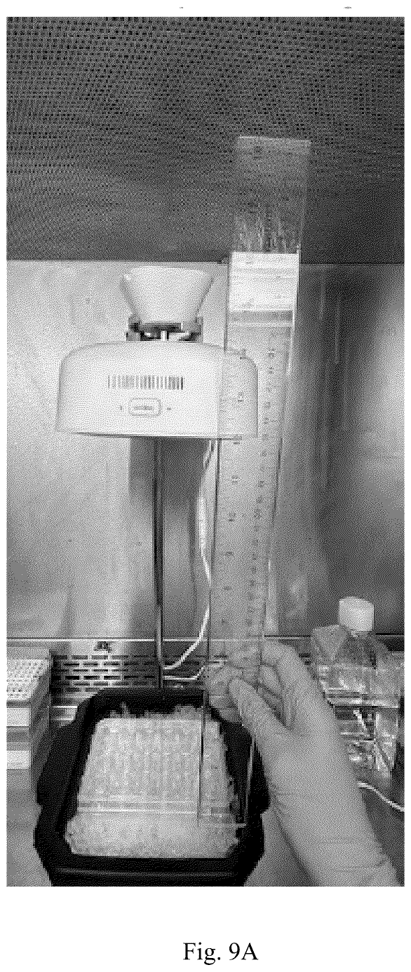

Referring to FIG. 8, there is shown an ultraviolet disinfection system, generally designated 1000, in accordance with an exemplary embodiment of the present invention. The system 1000 may be generally the same as system 100 discussed above except that the system 1000 may be configured to adjust the output of the light source(s) 1004 based on the presence of surfaceborne or airborne pathogens. The system 1000 may include a controller 1006, light source 1004 and subject detection sensor(s) 1008 each being generally the same as the controller 106, light source 104 and sensors 108 described above in relation to system 100. In some embodiments, the system 1000 includes one or more pathogen detection sensors 1009. The pathogen detection sensors 1009 may be sensors and/or systems in communication with the controller 1006 that are configured to detect the presence of surfaceborne and/or airborne pathogens within a predetermined range. In some embodiments, the pathogen detection sensor(s) 1009 are configured to measure the amount of surfaceborne and/or airborne pathogens within a predetermined space (e.g., a volume within range of the sensors 1009). In some embodiments, the pathogen detection sensor(s) 1009 are configured to detect surfaceborne and/or airborne pathogens whether alive, dead or inactive.

The controller 1006, light source 1004, one or more subject detection sensors 1008 and/or one or more of the pathogen detection sensors 1009 may be in communication with one another. In some embodiments, the controller 1006, light source 1004, one or more subject detection sensors 1008 and/or one or more of the pathogen detection sensors 1009 are coupled to a common housing (e.g., similar to housing 102 discussed above in relation to system 100). For example, a controller 1006, light source 1004, one or more of sensors 108 and/or 109 positioned within a common housing may be provided as a UV disinfection device similar to what is described above. In some embodiments, the pathogen detection sensors 1009 may be in communication with the controller 1006 and exterior to the housing. For example, the pathogen detection sensors 1009 may be external to a UV disinfection device and in communication therewith such that data may be transmitted from the sensors 1009 to the controller 1006. In some embodiments, the system 1000 includes a plurality of UV disinfection devices in communication with a common computing device (e.g., client device 1010), server, and/or network. Accordingly, the system 1000 may include a plurality of light sources 1004, controllers 1006, subject detection sensors 1008 and/or pathogen detection sensors 1009.

The system 1000 may be configured to control operation of the light source 1004 based on data generated by the subject detection sensor(s) 1008 and/or the pathogen detection sensor(s) 1009. The controller 1006 may be configured to adjust the intensity of a UV light (e.g., a far-UVC light) emitted by the light source 1004 based on data received from one or more of sensors 1008, 1009. In some embodiments, the controller 1006 is configured to adjust the intensity of the UV light emitted by light source 1004 based on detection data generated by the detection sensors 1008 in generally the same manner as discussed above with regards to system 100. In some embodiments, the controller 1006 is configured to receive pathogen detection data from the one or more pathogen detection sensors and adjust the UV light output by light source 1004.

For example, in response to receiving detection data from the sensors 1009 indicating the presence of pathogens, viruses, and/or bacteria, the controller 1006 is configured to cause the light source 1004 to emit or increase the intensity of emitted far-UVC light. As a further example, in response to receiving detection data from the sensors 1009 indicating that substantially no pathogens, viruses, and/or bacteria are detected, the controller 1006 is configured to cause the light source 1004 to cease emitting or decrease the intensity of emitted far-UVC light. In some embodiments, the controller 1006 is configured to receive detection data from sensors 1009 indicating a concentration of pathogens within a given space and adjust the intensity of UV light emitted by light source 1004. For example, in response to receiving detection data from the sensors 1009 indicating that a pathogen concentration is above a predetermined threshold, the controller 1006 is configured to cause the intensity of the far-UVC light emitted by light source 1004 to be increased.

In some embodiments, the system 1000 is configured to adjust the output of one or more light source(s) 1004 based on environmental data received at the controller 1006. For example, and as discussed above, the controller 1006 is configured to adjust output of the light source 1004 based on detection data received from sensors 1008 and/or 1009. In some embodiments, the controller 1006 may receive data positional data relating to the physical location of the light source(s) 1004 and control operation thereof based on the positional data. For example, positional data may include data indicating that a light source 1004 is installed within a HVAC system of a building and the controller 1006 is configured to control operation of the light source 1004 accordingly (e.g., continuous transmission of far-UVC light). In some embodiments, the environmental data may be based on weather/climate data based on the geographic location of light source 1004. In some embodiments, the controller 1006 is configured to receive environmental data indicating a reported outbreak of a pathogen in the vicinity of a light source 1004 and automatically cause the light source 1004 to emit and/or increase the intensity of an emitted far-UVC light. For example, weather data indicating an increase in humidity, typically associated with an increase in the likelihood of pathogen transmission in human environments, is transmitted to the controller 1006. In response to receiving said data, the controller 1006 may be configured to cause the intensity of the far-UVC light emitted by light source 1004 to be increased. The controller 1006 may be configured to receive data relating to a plurality of different environmental related factors (e.g., humidity, temperature) associated with the transmission of pathogens and adjust the intensity of the far-UVC light emitted by light source 1004 accordingly.

In some embodiments, the system 1000 is configured to optimize the intensity of the far-UVC light emitted by light source 1004 based on a plurality of different data points (e.g., a multivariate analysis). The controller 1006 may be configured to receive a plurality of data from different sources and adjust the intensity (e.g., increase, decrease) based on an analysis of an aggregation of the plurality of data. In some embodiments, the plurality of data may include, but is not limited to, any combination of: subject detection data, pathogen detection data, regulatory threshold data, public heath report data, geographic location data, infrastructure data, environmental data, and/or weather data. In some embodiments, the controller 1006 is configured to optimize the intensity of a plurality of light sources 1004 based on an aggregate of a plurality of the data from different sources. In some embodiments, the system 1000 of the present disclosure is configured to focus UV light and/or adjust the intensity thereof to areas where subjects were detected and/or that have an increased likelihood of pathogen or bacteria presence in generally the same manner as system 100 as discussed above.

In some embodiments, the system 100 and/or system 1000 are configured to aid in the generation of natural immunity and/or inoculation in a subject. In some embodiments, inactivation of pathogens (e.g., virus, bacteria) includes exposure of the chemical that bonds the microbe (e.g., mummification), replicate the pathogen billions of times in a cellular model until virulence loss, and/or isolate a unique protein, DNA fragment and/or section of the microbe (e.g., mRNA, Subunit, Toxoid). The systems 100 and/or 1000 may be configured to emit a UV light that creates chemical bonds between adjacent thymine nucleotides in DNA known as thymine dimers. Such bonds may block DNA replication. In some embodiments, the UV light emitted by systems 100 and/or 1000 (e.g., the far-UVC light) as discussed above is configured to reach microbe DNA and cause dimers (e.g., thymine dimers) while not affecting DNA in the protective nucleus of subjects (e.g., humans, animals).

The system 100 and/or system 1000 may be configured to provide inoculation similar to that of a vaccine in that the pathogens inactivated via the system 100 and/or system 1000 may imitate an infection when within the subject's body. The presence of such disease-causing organisms in the body may cause the subject's body to engage natural defenses. In vaccines, the active ingredient is an antigen, which causes the immune system of a subject to produce antibodies. In vaccines, the antigen may be a weakened, attenuated, or killed bacteria or virus, parts of the exterior surface or genetic material, and/or bacterial toxin treated to make it non-toxic. Immunity may be derived from natural exposure to pathogens or administration of vaccines. Combinations of natural immunity and vaccine-derived immunity in some instances is optimal. Immunity may not in all instances prevent disease, however immunity may limit the severity of disease. Such immunity provides protection at both an individual level and at a community level.

The UV light emitted by systems 100 and/or 1000 may inactivate or kill pathogens as discussed above. The pathogens may be, for example, naturally occurring or the result of exhalation or contamination by a subject (e.g., human, animal). Pathogens inactivated via systems 100 and/or 1000 may be ingested by a subject (e.g., via inhalation, physical touch, skin contact). For example, the systems 100 and/or 1000 are installed within a room such that surfaceborne and/or airborne pathogens are deactivated via the far-UVC light and inhaled by a subject within the room. In such instances, subjects receive a continual low-dose of immune exposure over long periods of time (e.g., over days, weeks, months, years). Inhalation of inactivated pathogens may provoke immune responses that lessen the impact of pathogens and provide some level of immunity to subjects. In some embodiments, the volume of the inactivated or killed pathogens ingested and/or breathed by a subject to provide natural immunity and/or inoculation in a subject may vary by pathogen. In some embodiments, the concentration, nature of pathogens (e.g., virus, or bacteria) and mean of infection (e.g., ingestion, or inspiration) affect the level and intensity of an immune response. In some embodiments, the frequency of exposure (e.g., continual exposure or sporadic acute exposure) affects the nature and intensity of an immune response. In some embodiments, the associated immunogenicity level is measured with a device.

In some embodiments, an immunogenic composition is prepared by the presently disclosed UVA, UVB, or UVC, including without limitation, far-UVC disinfection system to generate a vaccine composition. In some embodiments, the vaccine composition can be used to induce immunity against the encoded antigenic protein. Vaccines can be formulated using standard techniques and can comprise, in addition to a replication-incompetent adenovirus vector encoding a desired protein, a pharmaceutically acceptable vehicle, such as phosphate-buffered saline (PBS) or other buffers, as well as other components such as antibacterial and antifungal agents, isotonic and absorption delaying agents, adjuvants, and the like. In some embodiments vaccine compositions are administered in combination with one or more other vaccines. Dosage units of vaccine compositions can be provided. Such dosage units typically comprise 10⁸ to 10¹¹ particles (e.g., 10⁸, 5×10⁸, 10⁹, 5×10⁹, 10¹⁰, 5×10¹⁰, 10¹¹). In some embodiments, the dosage of 5×10¹⁰ virus particles is of choice. Particularly, a dosage (5×10¹⁰) is best suited for humans in clinical trials.

In some embodiments, the disclosure provides immunogenic compositions prepared by the disclosed far-UVC disinfection system, the compositions comprising pathogens rendered non replicative by far-UVC exposure while retaining antigenic epitopes sufficient to elicit an immune response upon administration to a subject.

In some embodiments, it is provided a method of triggering an immune response in a subject, the method comprising administering to the subject a therapeutically effective amount of the immunogenic composition prepared by the far-UVC disinfection system. In some embodiments, administration is by mucosal (including intranasal or oral), or epidermal routes, optionally with a pharmaceutically acceptable carrier and/or an adjuvant.

In some embodiments, provided herein is a method of preventing or treating a disease in a subject in need thereof, the method comprising administering to the subject the immunogenic composition prepared by far-UVC inactivation of a pathogen associated with the disease, in a prophylactically or therapeutically effective regimen. In some embodiments, the method reduces disease incidence, severity, pathogen load, or duration of symptoms.

In some embodiments, provided herein is a method of enhancing immunity in a subject, the method comprising administering the immunogenic composition to induce, boost, or broaden humoral and/or cellular immune responses specific to one or more antigens of the inactivated pathogen. In some embodiments, enhancement includes increasing antigen specific antibody titers, neutralizing activity, memory B cell responses, or T cell responses.

In certain embodiments, the pathogens subjected to far-UVC disinfection comprise viruses, bacteria, or a fungi, including but not limited to enveloped and non-enveloped viruses (e.g., influenza virus, respiratory syncytial virus, coronavirus, adenovirus), Gram positive and Gram-negative bacteria (e.g., Staphylococcus, Streptococcus, Escherichia), or fungal organisms (e.g., Cryptococcus). In some embodiments, the far-UVC disinfection system delivers radiation in the far-UVC spectrum sufficient to inactivate pathogen replication while preserving immunogenic structural proteins and epitopes.

In some embodiments, it is provided the use of inactivated pathogens produced by far UV treatment as whole pathogen antigens within a vaccine formulation, optionally combined with stabilizers, buffers, and adjuvants suitable for the selected route of administration.

In some embodiments, the present disclosure provides methods of inducing cross protective or strain transcending immunity by administering far-UVC inactivated, antigenically conserved pathogen preparations, optionally in prime boost regimens and optionally in combination with heterologous antigen platforms.

In further embodiments, the methods include administering one or more doses according to a schedule comprising a priming dose and one or more booster doses, with dosing intervals and quantities selected to achieve desired immunogenicity and clinical outcomes. In embodiments, the methods comprise administering the composition to a subject being for instance a pediatric, an adult, a geriatric, or an immunocompromised subject.

In some embodiments, the disclosed methods and compositions comprise a far-UVC inactivated pathogen where the inactivated pathogen is combined with one or more additional antigens to broaden coverage. In some embodiments, provided herein are combination therapies wherein the immunogenic composition is co administered with antivirals, antibacterials, or antifungals agents.

In some embodiments, a volume of air, volume of liquid, volume of vapors, and/or surface area comprises at least about 10⁴ to at least about 10¹⁵ inactivated, attenuated, weakened, dormant, or killed virus or particles thereof. In some embodiments, a volume of air, volume of liquid, volume of vapors, and/or surface area comprises at least about 10⁴, at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, or at least about 10¹⁵ inactivated, attenuated, weakened, dormant, or killed virus or particles thereof. In some embodiments, the volume of air, volume of liquid, volume of vapors, and/or surface area comprises at least about 5×10¹⁰ inactivated, attenuated, weakened, dormant, or killed virus or particles thereof.

### UV Light Inactivation of Microorganisms

Ultraviolet (UV) light is a high-energy form of electromagnetic radiation capable of disrupting microbial cells and viruses. The primary mechanism of UV disinfection is damage to nucleic acids (DNA or RNA) through photochemical reactions. UV photons (especially in the UVC range) are strongly absorbed by genetic material, causing adjacent pyrimidine bases (typically thymine in DNA, or uracil in RNA) to bond together abnormally. These UV-induced lesions—such as cyclobutane pyrimidine dimers (commonly thymine dimers) and 6-4 photoproducts—distort the DNA/RNA structure and block replication or gene expression. In essence, the microorganism can no longer reproduce or infect, achieving inactivation rather than instant “killing.” If the damage is extensive and not repaired by the cell's repair enzymes, the microbe becomes nonviable and cannot replicate. Thymine dimers can form in a DNA strand after UV exposure. These dimers create a kink in the DNA and block polymerases, which is a primary mechanism of UV-induced microbial inactivation (Browne, K. Appl. Microbiol. 2021, 1, 537-556).

UV can also damage other biomolecules like proteins, albeit to a lesser extent. For example, high doses of UV can disrupt a virus's protein capsid or denature enzymes in bacteria. Proteins absorb UV most strongly around 280 nm, so some slow secondary damage to proteins occurs, but nucleic-acid damage at ~260 nm is the dominant germicidal action. This means all types of microbes—bacteria, viruses, and molds (fungal cells/spores)—are susceptible because they all contain DNA or RNA targets. Importantly, spores (such as bacterial endospores and mold spores) have protective coats and dormant metabolism, making them more UV-resistant; a higher dose or longer exposure is required to penetrate these layers and achieve the same kill level as in vegetative bacteria or viruses. Nonetheless, given sufficient UV dose, all microbes can be inactivated, including antibiotic-resistant bacteria and hardy fungal spores. Another benefit is that UV targets fundamental cell components (nucleotides), so it inactivates microbes regardless of drug resistance or pathogenic traits (Browne, K. Appl. Microbiol. 2021, 1, 537-556).

UV disinfection is a surface or line-of-sight method. UV light cannot penetrate opaque materials, and even a thin film of organic matter (dirt, biofilm, dust) can shield microbes from UV exposure. Thus, effective UV treatment requires direct exposure of the microorganisms: surfaces should be pre-cleaned so that the UV can reach the target pathogens. Unlike chemical disinfectants, UV leaves no residue and works quickly (often seconds to minutes), but it also has no lasting residual effect—it inactivates only during the exposure period. For disinfection purposes, the UVC range (wavelengths ~200-280 nm) is the most germicidal, with efficacy peaking around 265 nm. Below, two specific UVC wavelengths commonly used for microbial disinfection: 254 nm and 222 nm are described with a focus on describing their effectiveness, uses, advantages, and limitations.

### Using 254 nm UV Light (Also Referred to as “Conventional UVC”)

UV at 254 nm lies in the germicidal UVC range and is the classic wavelength used in disinfection. Low-pressure mercury vapor lamps naturally emit UVC light predominantly at 254 nm, which is near the optimal wavelength for DNA absorption and microbial kill. Because of this, 254 nm UVC is highly effective at inactivating a broad spectrum of organisms—bacteria (including pathogens like Staphylococcus, E. coli), viruses (enveloped and non-enveloped), and fungi (yeasts and molds)—given appropriate doses. It has been widely applied in water purification, wastewater treatment, air and surface sterilization systems since the mid-20th century. From small bench-top UV sterilizers to whole-room disinfection units, 254 nm has demonstrated reliable performance in achieving 99.9%+ kill (3-4 log reductions or more) of microbes on exposed surfaces and in air. This wavelength's efficacy stems from its strong interaction with microbial DNA—254 nm is close to the 260 nm peak where nucleic acids absorb UV most intensely, thus readily forming DNA damage like thymine dimers that deactivate the organism (Browne, K. Appl. Microbiol. 2021, 1, 537-556).

### Using Novel 222 nm UV Light (Far-UVC)

222 nm UV can be used as a germicidal wavelength that is highly effective against microbes yet safer for human exposure. Far-UVC light (around 222 nm) is produced by krypton-chloride excimer lamps, which emit a narrow band of UV. Like 254 nm, the 222 nm wavelength is within the DNA absorption range and can cause direct DNA/RNA damage to microorganisms. 222 nm inhibits microbial genomes effectively by inducing DNA lesions (e.g. thymine dimers) similarly to 254 nm, inactivating bacteria, spores, and viruses in the same fundamental way (Taylor W, et al., Appl Environ Microbiol. 2020 Apr. 1; 86(8):e03039-19). However, a key difference is that 222 nm is even more strongly absorbed by proteins and other biomolecules than 254 nm. The absorbance of protein peptide bonds is about 20 times higher at 222 nm compared to 254 nm. This means far-UVC can damage microbial proteins (such as enzymes, structural proteins, and viral capsid proteins) to a significant extent. For example, critical enzymes that microbes rely on—including DNA repair enzymes like photolyase—can be directly inactivated by 222 nm exposure. This multi-target damage can enhance the overall efficacy of disinfection. In practical terms, pathogens exposed to 222 nm UV not only suffer DNA damage but also protein damage that can prevent them from repairing or surviving the initial injury. Bacteria irradiated with 222 nm have less ability to photoreactivate (repair UV lesions) than those irradiated with 254 nm, due to far-UVC's attack on the photorepair enzymes. Far-UVC may also induce oxidative damage (e.g. lipid peroxidation in cell membranes) that complements the direct photochemical damage (McDonald D. et al., (2024) PLOS Water 3(7): e0000238).

With appropriate doses, 222 nm light can achieve comparable log-reductions to 254 nm for bacteria (including drug-resistant strains), inactivate hardy bacterial spores, and efficiently neutralize viruses (Taylor W, et al., Appl Environ Microbiol. 2020 Apr. 1; 86(8):e03039-19). In some cases, 222 nm even outperforms 254 nm; for instance, certain viruses have been observed to be more readily inactivated by 222 nm UV than by conventional 254 nm UV under the same conditions (McDonald D. et al., (2024) PLOS Water 3(7): e0000238). Without wishing to be bound by any particular theory, this may be because viruses (especially non-enveloped viruses) often rely on protein capsids for survival outside a host, and far-UVC's protein damage compromises those structures. Some studies found 222 nm achieving similar or slightly higher inactivation rates than 254 nm, while others noted that a higher fluence (UV dose) of 222 nm was required to reach the same kill level for E. coli as 254 nm (McDonald D. et al., (2024) PLOS Water 3(7): e0000238). Without wishing to be bound by any particular theory, it is believed that 222 nm is highly effective for airborne and surface pathogens when sufficient dose is applied, and its added protein damage may give it an edge in preventing microbial recovery.

One of the most remarkable features of 222 nm UV is its safety profile for human exposure. Due to the physics of light-tissue interaction, 222 nm wavelengths are absorbed so strongly by the outermost layers of biological tissue that they cannot penetrate into the living cells of skin or eyes. The stratum corneum (outer dead-cell layer of skin) and the tear film/outer corneal layer in the eye absorb and attenuate 222 nm light, so very little reaches the sensitive basal skin cells or lens of the eye. In contrast, 254 nm (longer wavelength) penetrates deeper and can damage those tissues. Studies confirm that far-UVC light can be used at doses that kill microbes while causing no observable acute harm to mammalian skin or eyes. For example, researchers demonstrated that 222 nm UV could kill MRSA bacteria on a surface but produced almost no DNA damage in a 3D human skin model and did not cause skin inflammation in hairless mice (Buonanno M. et al., Radiat Res. 2017 April; 187(4):483-491). This opens up huge possibilities for deploying 222 nm disinfection in occupied spaces—something that traditional 254 nm germicidal lamps cannot safely do without protective equipment.

222 nm UV can be used in healthcare, public transport, and indoor environments. Far-UVC light can continuously sanitize air and surfaces even when people are present, for instance, ceiling-mounted 222 nm excimer lamps in occupied rooms can be used to disinfect air in real time. Overhead far-UVC lamps achieved >98% reduction of airborne microbes in a room within minutes, effectively cleaning the air to outdoor-equivalent safety levels while people were in the space (Eadie, E., Hiwar, et al., Sci Rep 12, 4373 (2022)). Potential uses include hospitals (to curb healthcare-associated infections by continuously bathing high-touch surfaces and room air in far-UVC), schools, airports, airplanes, offices, and other busy indoor areas where traditional UVGI would be unsafe. Far-UVC units can also be considered for sanitation of conveyor belts or food handling areas where workers are present, as well as in self-disinfecting elevator cabins and bathrooms. Beyond air and surface use, 222 nm is being explored for water disinfection as a mercury-free alternative to 254 nm lamps. It has been shown to inactivate waterborne pathogens effectively, and the reduced photoreactivation could be beneficial in keeping treated water safe (222 nm causes greater protein damage and repair inhibition of E. coli than 254 nm for water disinfection|PLOS Water). That said, using 222 nm in water may require addressing its higher absorption by organic matter and ensuring adequate dose. The technology is still relatively new, and standards (for both safety and performance) are being developed. Nonetheless, early deployments and research suggest 222 nm UV could revolutionize disinfection by enabling round-the-clock, human-safe antimicrobial protection in settings at high risk of pathogen spread.

Use of 222 nm UV is an exemplary embodiment, and the foregoing and subsequent description should not be construed as a suggestion that this particular wavelength must be used to practice the methods described herein. Rather, the methods may be practiced using any suitable ultraviolet wavelength(s), including but not limited to a wavelength(s) within the UVC, UVB, and UVA spectral ranges, or across any sub-range thereof. Such wavelength or range of wavelengths may be used individually, sequentially, or in combination. Selection of a wavelength or wavelengths may be based on the desired immunogenic response, target pathogen type, desired degree of inactivation or attenuation, exposure conditions, environmental context, safety thresholds, or other operational parameters. One of ordinary skill in the art will recognize that the selection of wavelength(s) may be varied without departing from the scope of the present disclosure.

In some embodiments, the ultraviolet light used to inactivate a pathogen (e.g., using a method as described herein) comprises UVA, UVB, or UVC light. In some embodiments, the ultraviolet light used to inactivate a pathogen (e.g., using a method as described herein) comprises UVA, UVB, UVC light, or any combination thereof. In some embodiments, the ultraviolet light comprises an output wavelength of from about 315 nm to about 400 nm. In some embodiments, the ultraviolet light comprises an output wavelength of from 315 nm to 400 nm. In some embodiments, the ultraviolet light comprises an output wavelength of from about 280 nm to about 315 nm. In some embodiments, the ultraviolet light comprises an output wavelength of from 280 nm to 315 nm. In some embodiments, the ultraviolet light comprises an output wavelength of from about 100 nm to about 280 nm. In some embodiments, the ultraviolet light comprises an output wavelength of from 100 nm to 280 nm. In some embodiments, the ultraviolet light comprises an output wavelength of from about 230 nm to about 280 nm. In some embodiments, the ultraviolet light comprises an output wavelength of from 230 nm to 280 nm. In some embodiments, the ultraviolet light comprises an output wavelength of from about 200 nm to about 230 nm. In some embodiments, the ultraviolet light comprises an output wavelength of from 200 nm to 230 nm. In some embodiments, the ultraviolet light comprises an output wavelength of from about 206 nm to about 230 nm. In some embodiments, the ultraviolet light comprises an output wavelength of from 206 nm to 230 nm. In some embodiments, ultraviolet light comprises UVC. In some embodiments, the ultraviolet light used to inactivate a pathogen (e.g., using a method as described herein) comprises far-UVC. In some embodiments, the ultraviolet light used to inactivate a pathogen (e.g., using a method as described herein) comprises an output wavelength of about 254 nm or 254 nm. In some embodiments, the ultraviolet light used to inactivate a pathogen (e.g., using a method as described herein) comprises an output wavelength of about 222 nm. In some embodiments, the ultraviolet light used to inactivate a pathogen (e.g., using a method as described herein) comprises an output wavelength of 222 nm.

### Primary Advantage of 222 nm Far-UVC

Safe for Occupied Spaces: A unique advantage of 222 nm is its limited penetration in human tissues. Properly filtered 222 nm lamps (which remove any longer wavelengths) can operate within current safety guidelines for skin/eye exposure, allowing continuous use around people (McDonald D. et al., (2024) PLOS Water 3(7): e0000238). This enables direct air and surface disinfection in inhabited environments—for example, one can install far-UVC lamps in ceilings or use portable units in rooms to continuously kill airborne microbes without harming occupants. This safety profile has been confirmed by multiple studies showing no significant skin DNA damage or acute eye injury at appropriate exposure levels (Buonanno M, et al., Radiat Res. 2017 April; 187(4):483-491).

254 nm and 222 nm UV each offer powerful disinfection capabilities, and both wavelengths leverage the same fundamental UV mechanisms (DNA damage leading to microbial inactivation (Browne, K. Appl. Microbiol. 2021, 1, 537-556), but with different use cases. 254 nm wavelength light is restricted to unoccupied spaces due to safety concerns. In contrast, 222 nm far-UVC is a novel approach that sacrifices a bit of efficiency for a vastly improved safety margin, enabling unique applications like continuous disinfection of occupied spaces.

### UV-Inactivated but Viable Airborne Pathogens

UV-C light is widely used to inactivate airborne bacteria, viruses, and fungi, but pathogens are not always completely destroyed. Microorganisms can survive UV exposure in a damaged or dormant state and remain viable, meaning they are not infectious at that moment but still alive.

Survival and Regaining Infectivity: A major concern is that some microbes survive UV disinfection and later recover their ability to infect. UV primarily damages microbial DNA/RNA, often leaving cells structurally intact. Studies show UV treatment can push bacteria into a dormant “viable but nonculturable” (VBNC) state instead of killing them, meaning they can't grow on lab media but are alive and may “wake up” later. For example, E. coli and P. aeruginosa exposed to UV went dormant but later revived and proliferated when conditions allowed. Many pathogens have evolved DNA repair mechanisms—photoreactivation (light-induced repair) or dark repair—that can reverse UV damage (Gonzilez, Y. et al., Sustainability 2023, 15, 11262). In one report, Legionella pneumophila bacteria nearly completely restored their infectivity after UV exposure when subsequently placed in visible light (Oguma K. et al., July 2004, Water Research 38(11):2757-63). This means UV-“killed” microbes can regain full virulence if given time or light to repair their genes. Such regrowth or reactivation of survivors is a known risk of UV disinfection (Wang M. et al., November 2020, Chemosphere 268(1)), potentially leading to unexpected disease transmission if a portion of an airborne pathogen population remains alive

Immune and Allergic Responses Triggered: When pathogens are truly inactivated (unable to replicate), their remaining particles can trigger immune responses in people who inhale them. Inflammatory reactions may occur as the body responds to pathogen components. For instance, inhaling bacterial endotoxin can cause a strong lung immune reaction—studies have noted dramatic increases in inflammatory cells in the airways when people are exposed to aerosolized endotoxin (Farokhi A. et al., Environ Health. 2018 Feb. 8; 17(1):14). Dead viral particles (or fragments thereof) can still be recognized by the immune system, potentially causing inflammation or flu-like symptoms even without an active infection (as seen with some vaccine responses). Allergic reactions are another concern: dead fungal spores remain fully capable of provoking allergies. The U.S. Environmental Protection Agency (EPA) recognizes that mold spores killed by UV can still trigger allergic responses and asthma symptoms. Both live and dead airborne allergens (like mold or dust mite debris) can cause illness in sensitive individuals, as such UV germicidal irradiation (UVGI) might not reduce allergy/asthma risks. Such immune stimulation means that air treated with UV could still pose health issues (cough, congestion, asthma, fever) if many inactivated microbes or their toxins are present, affecting indoor air quality and occupant health.

In some embodiments, the ultraviolet light is used to inactivate one or more pathogens (e.g., using a method as described herein) in a volume of air, a volume of liquid, a volume of vapors, and/or a surface area. In some embodiments, the volume of air, the volume of liquid, the volume of vapors, and/or the surface area contains inactivated pathogens after exposure to the ultraviolet light using a method as described herein. In some embodiments, the volume of air, the volume of liquid, the volume of vapors, and/or the surface area containing inactivated pathogens is used to produce an immunogenic composition. In some embodiments, the immunogenic composition is used to enhance immunity in a subject. In some embodiments, the immunogenic composition is used to preventing, treating, or reducing a disease or condition in a subject. In some embodiments, the immunogenic composition is used to stimulate an immune response in a cell population or tissue in a subject. In some embodiments, the immunogenic composition is used to trigger an immune response in a subject.

In some embodiments, the subject to which the immunogenic composition is administered (e.g., using any of the methods described herein) comprises an immune system. In some embodiments, the subject to which the immunogenic composition is administered (e.g., using any of the methods described herein) is an animal. In some embodiments, the subject to which the immunogenic composition is administered (e.g., using any of the methods described herein) is a mammal, a reptile, an amphibian, or a fish. In some embodiments, the subject to which the immunogenic composition is administered (e.g., using any of the methods described herein) is an undomesticated and/or a domesticated animal. In some embodiments, the subject to which the immunogenic composition is administered (e.g., using any of the methods described herein) is an farm animal, zoo animal, and/or a pet. In some embodiments, the subject to which the immunogenic composition is administered (e.g., using any of the methods described herein) is a mouse. In some embodiments, the subject to which the immunogenic composition is administered (e.g., using any of the methods described herein) is a human.

### Overview of Inhaled and Aerosolized Vaccines

Inhaled (aerosolized) vaccines are administered as sprays or aerosols to the respiratory tract (typically via the nose or mouth) instead of by injection. This needle-free delivery targets the mucosal immune system in the airways, aiming to generate immune defenses right at the site where many pathogens enter. By inducing immunity in the nose, throat, and lungs, inhaled vaccines can provide a first line of defense against infection that complements the systemic immunity induced by traditional intramuscular shots. They have been explored for a variety of diseases—especially respiratory infections—including COVID-19, tuberculosis, and influenza, among others (Gianchecchi E, et al., Influenza Other Respir Viruses. 2019 September; 13(5):429-437; Zhou M, et al., MedComm (2020). 2025 Jan. 16; 6(2):e70056).

### Mechanism of Action and Immune Response

Mucosal Immunity: Inhaled vaccines work by stimulating mucosal immune responses in the respiratory tract. They often induce secretion of IgA antibodies in the nasal and lung mucosa, which can neutralize viruses or bacteria at the entry site before infection is established. Secretory IgA can bind and block pathogens on the airway surfaces and even inside infected epithelial cells, preventing viral attachment or spread in the upper respiratory tract. In contrast, injected vaccines mainly elicit IgG antibodies in the blood, which provide “backup” protection if a pathogen spreads systemically. Because of this, inhaled vaccines are uniquely suited to prevent initial infection, while injected vaccines excel at preventing severe disease in the lungs (e.g. pneumonia) once a pathogen has invaded (Gianchecchi E, et al., Influenza Other Respir Viruses. 2019 September; 13(5):429-437).

Tissue-Resident Memory: Local memory immune cells can be generated in the respiratory mucosa. Inhaled vaccines can establish tissue-resident memory T cells and B cells in the lining of the airways, poised to respond rapidly upon pathogen encounter. These resident cells, along with mucosal IgA, provide localized protection at the site of entry that systemic vaccination alone often fails to achieve. By comparison, traditional intramuscular vaccines predominantly stimulate circulating IgG and T cells in the blood and lymphoid organs, with little effect on mucosal surfaces. Thus, the inhaled route can trigger both systemic immunity and mucosal immunity, offering a more comprehensive immune defense (including secretory IgA and tissue-resident cells in the respiratory tract in addition to the usual circulating antibodies and T cells) (Zhou M, et al., MedComm (2020). 2025 Jan. 16; 6(2):e70056).

Innate Immune Activation: Vaccines given by the respiratory route also engage local innate immune mechanisms. The act of delivering antigen to the airway can activate dendritic cells and macrophages in the mucosa, which then migrate to mucosa-associated lymphoid tissue (like nasal-associated lymphoid tissue, NALT) to initiate adaptive responses (Zhou M, et al., MedComm (2020). 2025 Jan. 16; 6(2):e70056). Some inhaled formulations may also leverage specialized epithelial “M cells” in the nasal passages to ferry vaccine antigens to underlying immune cells and jump-start mucosal immunity. Inhaled vaccines thus mimic certain aspects of natural respiratory infections (without causing disease), provoking a robust innate response followed by adaptive immunity that is tailored to the respiratory tract.

### Vaccine Platforms Suited to Inhalation

Live Attenuated Vaccines: weakened live pathogens can replicate in mucosal tissues without causing illness, making them suitable for intranasal delivery.

For instance, live attenuated influenza vaccine (LAIV) (e.g., FluMist®), which has ~80% efficacy in children and 40% in adults. LAIV replicates in the upper airways, inducing local IgA and systemic immunity. Other examples include Measles (via nebulizer) and Tuberculosis BCG (via aerosol). These vaccines stimulate mucosal T cells and IgA but require careful attenuation to ensure safety.

### Viral-Vectored Vaccines

Use non-replicating viral vectors (e.g., adenovirus, parainfluenza, poxvirus) to deliver antigens through nasal sprays or aerosols.

For instance, adenovirus-vectored COVID-19 vaccine, was tested as an aerosol mist inhaled through the mouth. These vaccines infect respiratory epithelial cells, driving strong mucosal immune responses similar to live viruses.

### Inactivated and Subunit Vaccines

Consist of killed whole-organism vaccines or protein subunits, often requiring mucosal adjuvants to enhance immunity.

For instance, dry-powder influenza vaccine (stabilized with inulin) induced strong immune responses in preclinical studies. Intranasal spike protein vaccines for COVID-19 (with CpG adjuvant) triggered strong mucosal IgA responses in mice (Inker J. et al., Innov Aging. 2021 Dec. 17; 5(Suppl 1):496).

Because soluble antigens are less immunogenic, these formulations use nanoparticles, emulsions, and adjuvants (e.g., CpG, LTB, cholera toxin B) to stimulate mucosal immunity.

### Live Bacterial Vectors

BCG (for tuberculosis) has been aerosolized as a TB booster and for research on controlled infection models (Fredsgaard-Jones T. et al., Front Immunol. 2024 Nov. 14; 15:1427371).

Researchers are exploring attenuated Bordetella pertussis (whooping cough) as an intranasal booster to reduce bacterial colonization in the respiratory tract.

Nucleic Acid Vaccines (mRNA/DNA)

Inhalable mRNA vaccines are under development but not yet approved. Scientists are testing lipid nanoparticle formulations that can be nebulized and inhaled (Inker J. Innov Aging. 2021 Dec. 17; 5(Suppl 1):496).

The goal is for airway cells to take up the mRNA and produce antigen locally, inducing strong lung-resident immunity. However, it can be challenging to ensure mRNA aerosol stability and uniform lung distribution.

DNA vaccines via nasal spray have been explored in animal models but require further optimization.

### Preclinical Studies and Immunogenicity Assays

Inhaled vaccine candidates are extensively tested in animal models (such as mice, ferrets, hamsters, and non-human primates) to evaluate safety, immunogenicity, and protection before human trials (Zhou M, et al., MedComm (2020). 2025 Jan. 16; 6(2):e70056). Mice are commonly used for initial tests, given their well-defined immune system and the availability of reagents to measure immune responses. Key immunogenicity assays employed in these studies include:

ELISA (Enzyme-Linked Immunosorbent Assay): to quantify antigen-specific antibodies in blood and mucosal secretions. For example, researchers measure IgG in serum and secretory IgA in bronchoalveolar lavage fluid or nasal washes after vaccination. ELISA provides titers of binding antibodies and is a staple for comparing immune responses elicited by different routes. (Zhou M, et al., MedComm (2020). 2025 Jan. 16; 6(2):e70056).

Neutralization Tests: to assess the functional ability of vaccine-induced antibodies to neutralize the live pathogen. This can be done via plaque reduction neutralization tests (PRNT) with live virus or pseudovirus neutralization assays. Neutralization is a critical measure for respiratory viruses like SARS-CoV-2 and influenza. In animal studies, serum (or even nasal wash fluid) from vaccinated subjects is incubated with virus to see if infection of cells is blocked. For instance, the above intranasal spike vaccine in mice generated IgA that neutralized multiple SARS-CoV-2 variants (Alpha, Beta, Gamma) much more effectively than serum IgG did, highlighting the broad protective potential of mucosal IgA (Zhou M, et al., MedComm (2020). 2025 Jan. 16; 6(2):e70056).

T Cell Assays: Cell-mediated immunity is evaluated using assays like IFN-γ ELISpot (which counts the number of T cells producing interferon-gamma in response to the antigen) or flow cytometry for intracellular cytokines. These assays determine the magnitude of vaccine-induced T helper and cytotoxic T cell responses. In a study of an inhaled tuberculosis vaccine (MVA85A vector) in mice and guinea pigs, T-cell responses in lung tissues were measured by ELISpot and found to be robust. Similarly, in mice immunized intranasally with a candidate vaccine, the frequency of IFN-γ secreting T cells in draining lymph nodes can be quantified to ensure a Th1-biased response (important for viruses and TB). Researchers also check the balance of antibody isotypes (e.g. IgG2a vs IgG1 in mice) to infer the Th1/Th2 bias—intranasal vaccines often aim for a Th1-dominant response to avoid the risk of Th2-mediated enhanced respiratory disease (Satti I, et al., Lancet Infect Dis. 2014; 14(10):939-946).

Organoid-Based Evaluations: studies are being performed using human organoid models to test inhaled vaccine efficacy in the lab. Nasal epithelium organoids—mini 3D cultures that mimic the nasal lining—can be grown to assess whether antibodies from vaccinated animals can protect human respiratory tissue. In one such experiment, live SARS-CoV-2 virus was added to nasal organoid cultures in the presence or absence of serum from vaccinated mice (Zhou M, et al., MedComm (2020). 2025 Jan. 16; 6(2):e70056). Virus pre-incubated with serum from mice given an experimental SARS-CoV-2 vaccine (a virus-like particle formulation) did not infect the organoids, similar to what was seen with a known neutralizing antibody, whereas virus mixed with serum from unvaccinated mice readily infected the cells (Zhou M, et al., MedComm (2020). 2025 January 16; 6(2):e70056). This organoid neutralization assay provides evidence that the inhalable vaccine induced functionally protective antibodies capable of blocking infection in human-like airway tissue. Such organoid models are valuable for evaluating mucosal vaccines because they closely replicate the environment of the human respiratory tract in vitro (Zhou M, et al., MedComm (2020). 2025 Jan. 16; 6(2):e70056).

Challenge Studies: In many animal experiments, vaccinated animals are deliberately infected with the target pathogen (e.g. live virus challenge) to test protection. Outcomes measured include reduction in viral or bacterial load in the lungs, prevention of disease symptoms, and histopathology of the lungs. For instance, mice vaccinated intranasally with a COVID-19 vaccine showed lower lung viral loads and less lung inflammation upon SARS-CoV-2 challenge compared to controls, correlating with their higher mucosal IgA levels (Zhou M, et al., MedComm (2020). 2025 Jan. 16; 6(2):e70056). Inhaled TB vaccine candidates in guinea pigs have been assessed by aerosol Mycobacterium tuberculosis challenge to see if they reduce TB lesions in the lungs. These challenge studies are critical for demonstrating the real-world effectiveness of mucosal vaccines in preventing infection or disease.

Animal studies show that inhaled vaccines can induce a combination of systemic and mucosal immune responses that often surpass those from intramuscular vaccination in the respiratory tract. By measuring antibodies (ELISA, neutralization), T cells (ELISpot, etc.), and even leveraging organoids or direct challenge, researchers gather comprehensive evidence of immunogenicity before moving these vaccines into human trials.

### Clinical Studies in Humans

### COVID-19 (SARS-CoV-2) Inhaled Vaccines

Adenovirus-Vectored Vaccines: CanSino Biologics developed an aerosolized version of their Ad5-nCoV vaccine, which demonstrated strong immune responses when inhaled. A Phase 3 trial in China found that using the aerosol vaccine as a booster induced stronger neutralizing antibody responses, including against Omicron, compared to a third injected dose. Li J X, et al., Lancet Infect Dis. 2023 October; 23(10):1143-1152).

Intranasal Vaccine Approvals: Bharat Biotech's iNCOVACC (BBV154), a chimpanzee adenovirus-based intranasal vaccine, was authorized in India in 2022. It showed broad immune responses, including mucosal IgA, which can reduce viral shedding and transmission.

Safety: A 2024 meta-analysis reviewed 15 studies and found inhaled vaccines to be well-tolerated, with minor side effects like transient throat irritation (Song G, et al., J Med Virol. 2024 April; 96(4):e29625).

### Tuberculosis (TB) Aerosol Vaccines

Aerosol BCG (Controlled Infection Model): Oxford scientists tested inhaled BCG as a controlled TB infection model in BCG-vaccinated adults. Even at high doses, no serious adverse events occurred, and the vaccine induced robust immune activation.

Aerosol Vaccine Candidates (MVA85A): The MVA85A vaccine, delivered via inhalation, induced stronger lung-localized immunity compared to injection, showing promise for respiratory TB protection.

Effectiveness: While no inhaled TB vaccine has yet demonstrated efficacy in preventing TB in large trials, animal models suggest aerosol BCG vaccination reduces TB burden in the lungs.

### Influenza (Intranasal Flu Vaccines)

Live Attenuated Influenza Vaccine (LAIV): The nasal spray flu vaccine has been widely used for decades, with ~80% efficacy in children and 40-50% in adults. It induces mucosal IgA, which helps prevent infection at the site of virus entry.

Safety: LAIV is well-tolerated with mild side effects like nasal congestion. It is not recommended for children under 2 years due to increased risk of wheezing.

Advancements: New approaches include adjuvanted intranasal flu vaccines and dry powder formulations for easier distribution and storage.

### Other Inhaled/Aerosol Vaccine Research

Measles: Large trials tested aerosol measles vaccines. While safe, the immune response in infants was slightly lower than injected vaccines, though booster doses in older children were highly effective.

Other Respiratory Viruses: Researchers are developing intranasal vaccines for RSV, parainfluenza, and pertussis, aiming to enhance mucosal immunity and reduce transmission. (PubMed)

Non-Respiratory Targets: Experimental research explores inhaled vaccines for Ebola, norovirus, and even cancer immunotherapy as a novel delivery approach.

### Aerosolized Vaccine Administration can Provide Enhanced Protection

Groundbreaking development of aerosolized vaccines has provided enhanced protection when targeting the respiratory tract. For instance, by utilizing intranasal and nebulization methods, COVID-19 vaccines demonstrated superior efficacy with lower doses compared to traditional intramuscular vaccines. As provided more in details below, investigations highlighted the stability, safety, and enhanced immune response of these vaccines, offering a promising single-dose, multivalent solution against various COVID-19 strains, including Omicron.

### 1.A: Aerosolized Vaccine Administration

Research increasingly indicates that the respiratory tract is not only the initial site of COVID-19 infection but also maintains the highest viral load throughout the illness and even in cases of Long COVID. This has led to the development of new vaccine classes designed to be administered intranasally or via nebulization. Importantly, the study of these vaccines has showed that much lower doses are required to achieve a similar level of protection as those conferred by conventional intramuscularly administered anti-virals. (Jearanaiwitayakul T. et al., 2024).

The investigations of Jearanaiwitayakul and colleagues demonstrated that nanoparticle chassis encapsulated in a 1-4 μm microcapsule are stable for up to 42 days and safe in mammalian and non-human primate test subjects. The vaccine presented consistent antigen release into the alveoli for up to five weeks following administration. It resulted in the highest immunoglobulin and memory T cell activation of all tested vaccination regimes.

This vaccine was found to be both single dose and multivalent and unlike traditional vaccines, it maintained stable efficacy against both conventional and Omicron COVID-19 strains. Additionally, this vaccine requires only a single administration, unlike most previous vaccines, including many aerosol-based ones. (Jearanaiwitayakul T. et al., 2024).

Furthermore, this inhaled and aerosolized vaccine treatment contained additives that may enhance its efficacy.

1.B: Nasal Sprays vs. Aerosolized

Other studies demonstrated that nasal sprays can be an alternative and additional intervention to prevent COVID-19 transmission as the first-line defense to the virus. Many advantages can be related to the use of a nasal spray vaccine or therapeutics, including but not limited to it being noninvasive, triggering local immunity, reducing systemic side effects, and feasible to be self-administered. (Xi et al., 2021).

### 1.C: Intranasal Efficacy for COVID Vaccine

When used as a booster for individuals previously vaccinated with inactivated COVID-19 vaccines, a single dose of intranasal COVID vaccine was showed to provide short-term protection against infections requiring medical attention. Research data indicated that a single dose of intranasal COVID vaccine was effective against symptomatic SARS-CoV-2 infections as a primary immunization for those without prior exposure. (Hongfei et al., 2024)

### 2.A: Test Inhaled Vaccines on Mice

Research on inhaled COVID vaccine showed that mice that received intranasal cGAMP-adjuvanted bivalent inhaled vaccine elicited robust and durable antibody responses. The stimulated antibody broadly neutralized the ancestral strain and variants of concerns (delta and omicron BA.1) in the upper and lower respiratory tracts (Jearanaiwitayakul T. et al., 2024). Furthermore, the immunized mice developed T-cell response in their lung tissue. Importantly, results showed that intranasal immunization with the inhaled vaccine candidate efficiently protected mice from nasal infection caused by COVID variants (e.g., both Wuhan-1 and BA.1 viruses) and even if some still got infected, their infection was mild and they did not develop any symptoms. This is because activated responses in the nasal cavity significantly suppressed virus production. Essentially, this investigation showed that nasal vaccine completely protected the mice from disease development and mortality (Jearanaiwitayakul T. et al., 2024).

### 2.B: Alternative Testing

Alternative investigations of interest to assess involve (1) immunogenicity assays and (2) testing on nasal (or other) organoids to understand immunogenic impact.

3: Allometric Analysis for Impact Transfer from Mice to Humans

Caution should be used when scaling doses from data generated by direct instillation methods, as drugs tend to be more potent when delivered by aerosol inhalation compared to direct instillation methods (Cooper et al., 2012). Direct instillation methods including: intranasal (Siddiqui et al., 2008) or intratracheal liquid (Brain et al., 1976, Liu et al., 2013) and spray instillation (Bivas-Benita, Zwier, Junginger, & Borchard, 2005) or dry-powder insufflation (Guillon et al., 2012,Morello et al., 2009) can be used as a screening tool to determine the approximate dose range for later inhalation studies, or to determine the ranking of efficacy/toxicity for a series of structurally similar drugs (Pauluhn & Mohr, 2000).

### 4: Testing Polyvalence

In some embodiments, it can be relevant for a disclosed vaccine of interest, such as but not limited to a vaccine being both single-dose and multivalent, to assess its efficacy vis-à-vis (1) different pathogens, and (2) different versions of each pathogen.

### 5: Exposure to Sub-Infectious Doses

Low-dose viral particles entering the respiratory and intestinal tracts may be able to induce T cell memory in the absence of inflammation, potentially resulting in different degrees of immunization. In this scenario, non-pharmaceutical interventions would play a double role, one in the short term by reducing the detrimental spreading of SARS-CoV-2 particles, and one in the long term by allowing the development of a widespread (although heterogeneous and uncontrollable) form of immune protection (De Angelis M L, et al., Viruses. 2021 May 22; 13(6):961).

Initial Dose of virus exposure is important in understanding severity of an infection (Wim Van Damme et al., Medical Hypotheses, Vol. 146, 2021, Article 110431; Karimzadeh S, et al., Epidemiol Infect. 2021 Apr. 14; 149:e96).

In some embodiments, low-dose UV-inactivated viral particles are used herein to induce T cell memory in the absence of inflammation.

### Overview of Effectiveness and Safety Summary

Inhaled and intranasal vaccines can generate unique immune profiles—especially inducing mucosal IgA antibodies and tissue-resident T cells—that conventional shots do not. For diseases like COVID-19 and influenza that spread via droplets and aerosols, this could translate to improved prevention of infection and transmission (not just mitigation of disease severity).

Effectiveness: Inhaled vaccines can effectively boost immunity. COVID-19 trials demonstrated that mucosal boosters can significantly raise neutralizing antibody levels (even against escape variants) and add mucosal IgA on top of systemic immunity (Zhang et al., Front. Immunol. (2023)). Influenza LAIV is effective in children and contributes to herd immunity by reducing community transmission. TB aerosol vaccine trials have shown immunogenicity in the lungs, which is expected to correlate with better protection (efficacy trials are the next step). However, there have been instances where inhaled vaccines were less effective than injections—as seen with primary measles immunization (slightly lower seroconversion) (Low N, et al., N Engl J Med. 2015; 372(16):1519-1529), or in some early COVID intranasal vaccine attempts (certain intranasal formulations in development did not achieve as high systemic antibody levels as the mRNA shots, possibly necessitating prime-boost regimens or higher doses). These examples highlight that the route of delivery can influence the magnitude of response, and formulation and dosing need to be optimized for inhaled vaccines to match or exceed the traditional approach.

Safety: The safety record of inhaled vaccines in trials has been largely positive. No severe adverse events have been attributed to the route of administration in the studies conducted. Local reactions (like nasal congestion, mild cough, throat irritation) are common but generally short-lived (Fredsgaard-Jones et al., Front. Immunol. (2024); Satti et al., Lancet Infect Dis (2014)). Systemic side effects (fever, fatigue, headache) occur at rates similar to or lower than injectable vaccines in most trials (Fredsgaard-Jones et al., Front. Immunol. (2024); Song et al., J. Med. Virology (2024)). An important safety consideration is ensuring the vaccines do not cause enhanced disease or excessive inflammation: so far, studies like the intranasal COVID vaccine in mice actually showed less lung inflammation upon virus challenge compared to some injected vaccines, owing to a more balanced Th1 response (Suzuki et al., Cell Reports Med (2022)). Live attenuated nasal vaccines like LAIV have specific contraindications (e.g. immunocompromised individuals, infants under 2 for flu) purely as precautions, but in indicated populations they have an excellent safety profile (LAIV does not revert to wild type and only rarely transmits to close contacts) (Mohn K G, et al., Hum Vaccin Immunother. 2018 Mar. 4; 14(3):571-578). For aerosol BCG, which involves giving people a live bacterium in the lung, careful dose escalation in the Oxford study showed it was remarkably safe even at high doses (Fredsgaard-Jones et al., Front. Immunol. (2024)). Nonetheless, each new inhaled vaccine must be rigorously tested, since the respiratory route can theoretically introduce risks like bronchospasm or unknown inflammatory reactions. Delivery devices (nebulizers, nasal sprayers) also need to be user-friendly and deliver consistent doses.

In conclusion, inhaled and aerosolized vaccines represent a cutting-edge strategy in vaccinology. They leverage the full breadth of the immune system by engaging mucosal defenses that standard injections miss. From COVID-19 to tuberculosis to influenza—and even legacy diseases like measles—these vaccines are being developed to improve protection where it's needed most: at the pathogen's point of entry. Ongoing clinical trials will guide further this approach, but the data so far (robust IgA, tissue-resident T cells, strong booster effects, and good safety) signal that the “next generation” of vaccines might indeed be a spray or a puff instead of a shot (Zhang et al., Front. Immunol. (2023); Song et al., J. Med. Virology (2024)).

### Impact of Subinfectious Doses on the Immune System

A subinfectious dose is an exposure to a virus or bacterium that is too low to cause an actual infection or noticeable illness. Such low-dose exposures can happen naturally (e.g. inhaling a few viral particles from the environment) or through controlled means like vaccines (which use inactivated or weakened pathogens). Even though they don't produce full-blown infection, these tiny exposures can still interact with the immune system. They can shape immune memory, trigger antibody production, or sometimes induce tolerance, depending on the context. Provided below herein is a summary of how subinfectious doses affect systemic and mucosal immunity, covering immune memory, priming vs. tolerance, cross-protection, secretory IgA, and cellular (T-cell) responses.

### Immune Memory and Antibody Responses

Even minimal exposures can prompt the adaptive immune system to form memory B cells and T cells, leading to antibody production without illness. For example, studies in primates showed that mucosal exposure to a subinfectious amount of SIV (a simian virus) still primed the immune system—monkeys developed virus-specific T-cell responses and antibody-secreting B cells in both blood and gut tissues (Polyanskaya N, et al., Virology. 2001; 279(2):527-538.). Similarly, in mice, a tiny inoculum of Helicobacter pylori bacteria (too low to cause disease) was enough to “prime” immunity: mice pre-exposed to a subinfectious dose had significantly lower stomach bacterial loads when later given a normal infectious challenge (Radcliff F J, et al., Infect Immun. 2001; 69(8):5186-5188). These cases show that a small antigen dose can create immunological memory and partial protection.

In vaccines, this principle is applied deliberately. Inactivated or attenuated vaccines give a controlled micro-exposure that elicits antibodies and memory cells without causing illness. Notably, even fractional vaccine doses can be effective. For instance, using a reduced dose (one-fifth) of yellow fever vaccine still induced seroconversion (neutralizing antibodies) in virtually all children, non-inferior to a full dose (Juan-Giner A, et al., Lancet Infect Dis. 2023; 23(8):965-973). Repeated low-dose immunizations (e.g. multiple shots or booster doses) are often used to gradually build up strong antibody levels and long-lived memory. Likewise, natural low-level exposures in the environment can serve as “boosters”—for example, someone who had a virus in the past might encounter traces of it again and mount a quick memory response, quietly boosting their antibody titers without any symptoms.

Immune Priming vs. Immune Tolerance

Sub-infectious exposures can have dual outcomes: in many cases they prime the immune system for future defense, but in other cases they induce a form of immune tolerance that dampens future responses. Which outcome occurs depends on factors like the dose, frequency, and whether the exposure triggers inflammation:

Immune Priming (Heightened Readiness): If a low-dose exposure provides enough antigen and includes danger signals (e.g. some tissue irritation or innate immune activation), it can activate specific lymphocytes and create memory. The next time the person sees that pathogen, their immune system responds faster and stronger. For instance, the primate and mouse studies above demonstrate priming—those low-dose exposures led to an enhanced ability to fight the pathogen later (Radcliff F J, et al., Infect Immun. 2001; 69(8):5186-5188; Polyanskaya N, et al., Virology. 2001; 279(2):527-538). In general, a small “preview” of an antigen educates the immune system, much like a mini-vaccine, so that it is not caught completely off guard later.

Immune Tolerance (Dampened Response): If the low-dose exposure fails to trigger a sufficient innate immune reaction, the immune system may become desensitized to that antigen instead of reactive. Research in a hepatitis C virus (HCV) model illustrates this outcome. Chimpanzees given repeated trace amounts of HCV (too low to cause infection) did develop HCV-specific T-cells, but these were skewed toward a regulatory, non-responsive phenotype (Park S H, et al., Nat Med. 2013 December; 19(12):1638-42). Essentially, the tiny exposures predisposed the host to tolerance: they expanded regulatory T cells (Tregs) that later suppressed the normal virus-fighting T-cells during a real infection (Park S H, et al., Nat Med. 2013 December; 19(12):1638-42). Because the initial exposure caused little to no inflammation, the immune system treated it more like a harmless encounter. In immunology, it's known that repeated low-dose antigen exposure is optimal for inducing Tregs (which secrete IL-10, TGF-β and suppress immune responses) (Vickery B P, et al., J Allergy Clin Immunol. 2011 March; 127(3):576-84). This mechanism is beneficial in contexts like preventing overreaction to gut microbes or food antigens, but in the case of a pathogen it can backfire by blunting protective immunity.

Notably, the line between priming and tolerance can depend on dosage thresholds—the immune system often shows a hormetic response. Low-level stimulation tends to be activating, whereas high-dose or constant stimulation can switch to a shut-down mode to prevent damage. This has been observed in innate immunity: a small dose of an endotoxin (like LPS from bacteria) will “train” innate immune cells to respond more vigorously next time, but a high dose of the same endotoxin can make those cells enter a tolerant state with reduced cytokine output (Lajqi T, et al., Biomedicines. 2023 Mar. 2; 11(3):766). In short, sub-infectious exposures sometimes act as an alarm drill (priming memory cells), and other times as a signal to stand down (inducing tolerance), depending on how the immune system interprets the context.

### Cross-Protection and Trained Immunity

Low-dose exposures can sometimes confer broader immunity than just against that single pathogen. This can happen via cross-reactive adaptive immunity or via generalized training of innate immunity:

Heterologous (Cross-Protective) Adaptive Immunity: An immune response sparked by one microbe can incidentally protect against a different (but related) microbe. Mild or subclinical infections often leave behind cross-reactive T-cells or antibodies that recognize similar antigens on other pathogens. For example, research on coronaviruses found that a prior coronavirus infection (such as a common cold strain) conferred partial protection against a different coronavirus later on. In that study, mice infected with a common cold coronavirus were less susceptible to a subsequent SARS-CoV-2 infection, thanks to cross-reactive immune memory. Likewise, people have shown cross-reactive T cells from past endemic coronavirus exposure that may have mitigated COVID-19 severity (Dangi T., et al., J Clin Invest. 2021; 131(24):e151969). These cases show that a small exposure to one pathogen might arm the immune system with tools that incidentally recognize a related pathogen. Vaccinology has even leveraged dose-timing to enhance this effect: an interesting example was the Oxford/AstraZeneca COVID-19 vaccine trial, where a regimen using a lower first dose followed by a standard second dose unexpectedly produced a higher efficacy (~90%) than two full doses (~70%) (Prakash S. et al, Front. Immunol., 21 Jan. 2024). One hypothesis is that the lower priming dose reduced immune system “overload” and allowed a broader, more effective T-cell response upon boosting—essentially tuning the adaptive response in a favorable way.

Trained Innate Immunity: Apart from specific antibodies and T-cells, even the innate immune system (traditionally thought to have no memory) can be “trained” by an initial exposure. A small exposure to microbial components can reprogram innate cells (like monocytes, macrophages, NK cells) to respond more robustly to any infection later. A classic example is the BCG vaccine (an attenuated Mycobacterium given against TB): children vaccinated with BCG not only gain some TB immunity but also had lower rates of unrelated infections and overall child mortality, suggesting a broad protective effect. Studies confirm that BCG and even measles vaccine induce this non-specific resistance, partly via epigenetic changes in innate immune cells. In essence, the initial low-level infection (or vaccine) puts the innate immune system on alert long-term. This cross-protection can be quite valuable—for instance, in communities with high vaccination, even those who haven't encountered a particular pathogen might be somewhat protected indirectly because their innate immunity is generally bolstered. However, as noted earlier, dose matters here too: repeated moderate stimulation trains innate cells to be extra responsive, whereas excessive or continuous stimulation can drive innate tolerance (cells start to deactivate to prevent chronic inflammation) (Lajqi T. et al., Biomedicines. 2023 Mar. 2; 11(3):766).

Overall, subinfectious exposures can have ripple effects beyond the specific pathogen, enhancing general antiviral or antibacterial readiness. This concept is being explored to design cross-protective vaccines and to understand phenomena like the hygiene hypothesis (how early-life microbe exposures might lower rates of allergies and autoimmune disease by training the immune system appropriately).

### Mucosal Immune Defenses and Secretory IgA

Mucosal immunity: antigens crossing the epithelium stimulate B and T cells in organized lymphoid follicles (Peyer's patches). Activated B cells become plasma cells that secrete polymeric IgA, which is transported across epithelial cells into the mucus layer. Repeated subinfectious exposures at mucosal surfaces primarily boost such local IgA and T-cell defenses at the pathogen's entry point.

Mucosal surfaces (such as the lining of the respiratory, gastrointestinal, or urogenital tracts) have specialized immune defenses that can be activated by tiny exposures. A key player is secretory IgA (SIgA), the main antibody type found in mucus and secretions. SIgA can neutralize viruses and bacteria at the site of entry, preventing them from ever establishing an infection. Even a subinfectious exposure that deposits a few antigens on these surfaces can induce a localized IgA response without causing systemic illness. For instance, during the COVID-19 pandemic, some people who were exposed to the virus never became PCR-positive or seropositive for IgG, yet they developed virus-specific IgA in their nasal mucosa (Chwa, J. S. et al., Viruses 2024, 16, 852). This suggests their mucosal immune system intercepted and cleared the virus early. In general, SIgA works by “immune exclusion”—it binds pathogens in the mucus, blocking them from attaching to or penetrating the epithelium (Vickery B P, et al., J Allergy Clin Immunol. 2011 March; 127(3):576-84). By neutralizing threats outside the body tissues, IgA prevents further immune activation. Thus, frequent low-dose exposures at mucosal surfaces (like inhaling small doses of airborne microbes) tend to strengthen this front-line defense, increasing IgA-producing plasma cells and mucosal memory B cells that are ready for the next encounter.

Importantly, mucosal exposure often stimulates local immunity more than systemic. For example, oral polio vaccine (a controlled attenuated exposure in the gut) generates strong IgA in the intestines, which protects against wild poliovirus infection at that entry portal. Intranasal flu vaccines likewise induce IgA in the nasal passages. These mucosal antibodies are critical because they can stop a pathogen before it spreads; they act as a first filter. Repeated low-dose exposures via the mucosa may also foster mucosal T cells that reside in the lining and can rapidly orchestrate an immune response if the pathogen tries to invade. In some cases, mucosal low-dose exposure can even induce mucosal tolerance—for example, oral tolerance to dietary antigens involves IgA and regulatory cells preventing an immune attack on food proteins (Vickery B P, et al., J Allergy Clin Immunol. 2011 March; 127(3):576-84). The immune system smartly balances aggression and tolerance on mucosal surfaces, since these are constantly bombarded with foreign substances. Overall, subinfectious antigen doses at mucosal sites predominantly boost secretory IgA and local immunity, which protects the host quietly and efficiently at the point of entry.

### Cellular Immune Responses and T-Cell Regulation

Low-dose exposures influence cellular immunity—the responses of T cells and other immune cells—in nuanced ways. If the exposure is sufficient to activate dendritic cells and co-stimulate T lymphocytes, it can generate effector T cells and memory T cells specific to the pathogen. These T cells might not be numerous (since the exposure was limited), but they patrol the body (and some take up residence in tissues) ready to spring into action on re-exposure. In the SIV monkey study mentioned, for example, subinfectious rectal virus exposure led to detectable virus-specific T-cell responses in both systemic circulation and the gut mucosa (Polyanskaya N, et al., Virology. 2001; 279(2):527-538). Those T cells are a cadre of sentinels induced without an actual infection. Memory T cells formed in such a way can be long-lived and, upon a real infection later, can quickly proliferate and coordinate viral clearance, often reducing disease severity.

However, as noted, low-dose antigen exposure can also selectively activate regulatory T cells (Tregs) under certain conditions. Tregs are CD4⁺ T cells that suppress other immune cells and promote tolerance. Repeated tiny exposures that lack “danger” signals (e.g. minimal tissue damage or inflammation) tend to favor the induction of Tregs over effector T cells. The HCV study in chimpanzees vividly demonstrated this: those trace viral exposures caused an increase in FoxP3⁺ regulatory T cells, which in turn blunted the normal T-cell attack when the animal later faced a higher dose infection. Essentially, the would-be warrior T cells were held in check by the regulatory cells induced by the prior low-dose exposure. Mechanistically, scientists think this happens because insufficient co-stimulation or inflammatory cytokines during the initial exposure leads T cells to default to a regulatory or anergic state (Park S H, et al., Nat Med. 2013 December; 19(12):1638-42). It's akin to showing the immune system an antigen in a context that whispers “this is not dangerous”—the immune response then either ignores it or actively suppresses reactions to it. This is beneficial for avoiding damage from harmless antigens, but detrimental if the antigen is actually a pathogen that one might encounter in force later.

Aside from T cells, innate cellular responses are also modulated by low-dose exposures. A small dose can “tune” innate cells—for example, a brief encounter with a tiny amount of endotoxin can cause macrophages to up-regulate certain receptors or epigenetic programs, so that a later infection triggers a faster, stronger release of inflammatory cytokines than untrained cells would produce. This phenomena (trained innate immunity) involves adjustments in cell metabolism and gene expression that make the cells more reactive. Conversely, if cells are continuously bathed in antigen (even at low level) or hit with a high dose initially, they can enter a state of innate tolerance, where they down-regulate pro-inflammatory signals and increase anti-inflammatory factors like IL-10 (Lajqi T, et al., Biomedicines. 2023 Mar. 2; 11(3):766). For example, endotoxin tolerance is a known effect where after an initial high LPS exposure, monocytes become transiently unresponsive to avoid runaway inflammation. In summary, the cellular arm of immunity responds to subinfectious doses on a spectrum: at one end, activating protective effector cells (both T cells and innate cells) and at the other, inducing regulatory mechanisms (like Tregs or tolerized macrophages) that curb responses. The outcome hinges on the presence of danger signals and the total antigen load seen by those cells.

Various repeated or low-dose exposure studies have been carried out. These include, for instance, the following:

Priming Immunity in Bacterial Infection: Mice primed with a subinfectious dose of H. pylori (gastric bacteria) had much lower bacterial counts after a later high-dose challenge than unprimed mice (Radcliff F J, et al., Infect Immun. 2001; 69(8):5186-5188). This indicates that the low-dose exposure successfully trained their immune system to fight off the infection, even though the initial exposure by itself didn't cause illness.

Local Virus Exposure Without Infection: Rhesus macaques exposed rectally to very low doses of SIV (an HIV-like virus) did not get overtly infected, yet they developed virus-specific T cells and antibody-secreting cells in both systemic and mucosal compartments (Polyanskaya N, et al., Virology. 2001; 279(2):527-538). The subinfectious exposure primed both systemic and local immunity, showing that even without detectable infection, the immune system recognized the virus and prepared defenses.

Tolerogenic Effect of Repeated Low Viral Doses: In a chimpanzee model, investigators found that exposing the animals to ultralow doses of HCV over time caused an expansion of regulatory T cells and an absence of effective protective immunity (Park S H, et al., Nat Med. 2013 December; 19(12):1638-42). When these animals were later given a normal infectious dose of HCV, their immune systems responded poorly—the prior low-dose encounters had induced a form of specific tolerance that made them less able to fight the virus (Park S H, et al., Nat Med. 2013 December; 19(12):1638-42). This highlights how repeated minute exposures can sometimes hinder, rather than help, immune defense by programming the immune response to stay subdued.

“Silent” T-Cell Responses in Humans: There is evidence that humans sometimes develop immune responses from subinfectious exposures in real life. Healthcare workers who experienced accidental needlestick exposures to HCV (often an extremely minute exposure) were found to develop HCV-specific T cell responses without ever having detectable viremia or antibodies in their blood. In other words, their T cells “saw” the virus and reacted, even though the virus never established a measurable infection. However, such responses may or may not confer full protection. In the HCV example, researchers caution that T-cell memory generated without an actual infection (and without antibody production) might not be robust enough to prevent future infection (Park S H, et al., Nat Med. 2013 December; 19(12):1638-42).—it could require a proper infection (with inflammation and antibody response) to develop truly protective immunity.

Broad Protection via Innate Training: Large population studies have noted that certain live vaccines or repeated low-grade infections correlate with lower incidence of unrelated illnesses. For instance, children in West Africa who received the BCG vaccine had better survival rates and fewer infections from various causes than unvaccinated children, beyond just tuberculosis protection. This non-specific benefit is attributed to trained innate immunity. The small, controlled infection from the vaccine effectively “boosts” the innate immune system in a way that provides cross-protection against other pathogens. Similarly, epidemiological data on measles vaccination hinted at reductions in overall child mortality not fully explained by preventing measles alone (Lajqi T, et al., Biomedicines. 2023 Mar. 2; 11(3):766.). These examples from the field suggest that repeated low-level exposures (whether via vaccines or natural microbe encounters) can calibrate the immune system to be more resilient in general.

In summary, sub-infectious doses of viruses or bacteria can have diverse effects on immunity. They often act as natural micro-immunizations—strengthening immune memory, stimulating antibodies (especially IgA on mucosa), and even broadening protection to other pathogens. Yet, if the context is not right, they can also lead to a dampened state, teaching the immune system to tolerate the antigen to avoid unnecessary inflammation. The balance between priming and tolerance, between local (mucosal) and systemic responses, and between adaptive and innate training, all depends on the details of exposure. Understanding these nuances helps in designing better vaccines (e.g. optimal dosing schedules, intranasal/oral vaccines for mucosal immunity) and in explaining how everyday low-dose microbial exposures keep our immune system educated throughout life.

### Combination and Multivalent Vaccines

Combination and multivalent vaccines stimulate the immune system in essentially the same way as traditional monovalent vaccines, but with multiple antigenic targets. Each antigen in the vaccine (whether from different pathogens in a combination vaccine or multiple strains in a multivalent vaccine) is taken up by antigen-presenting cells and presented to T cells, which then activate B cells specific to that antigen. This results in the production of antibodies and memory B/T cells against each included disease or strain. In some cases, one component can influence the response to another—for example, adding whole-cell pertussis to a diphtheria-tetanus vaccine was found to boost diphtheria antitoxin levels (a beneficial adjuvant effect), whereas using a less immunogenic pertussis component led to slightly lower diphtheria antibody levels.

Importantly, the human immune system is capable of responding to many antigens at once without being “overloaded”—even infants can theoretically handle thousands of immunologic challenges simultaneously. Studies have confirmed that giving multiple vaccines together does not overwhelm the immune system or reduce the response to each one in any clinically significant way (Elliman D, et al., 2003 May 10; 326(7397):995-6; Mazunina E P, et al., Vaccines (Basel). 2024 Oct. 24; 12(11):1206).

### Challenges for Multivalent Vaccines:

### 1. Formulation and Compatibility

Each component in a combination vaccine must remain stable and effective within the same vial. Chemical or physical incompatibilities between vaccine ingredients-such as antigen degradation or differing storage requirements-pose significant challenges. Developers must ensure that additives like adjuvants and preservatives do not affect efficacy or safety. This complexity makes the research and development (R&D) process longer and more demanding than for single-antigen vaccines.

### 2. Immunologic Interference

Combining multiple antigens may lead to immune interference, where one antigen's response is weakened due to competition within the immune system. For instance, early studies showed that combining whole-cell DTP (diphtheria-tetanus-pertussis) with Hib (Haemophilus influenzae type b) vaccine slightly reduced Hib antibody levels compared to separate administration. Manufacturers address this by adjusting antigen quantities or using different carrier proteins/adjuvants to ensure each component elicits a strong immune response.

### 3. Safety and Reactogenicity

Some combination vaccines have higher rates of common side effects like fever. In rare cases, high fever can trigger febrile seizures in young children. For example, the MMRV (measles-mumps-rubella-varicella) vaccine doubles the risk of febrile seizures compared to giving MMR and varicella separately, though the absolute risk remains low. Similarly, certain DTaP-based combinations showed a slight increase in fever-induced seizures. Despite this, rigorous clinical trials confirm that combination vaccines are as safe as individual vaccines.

### 4. Attribution of Adverse Events

Determining which component caused a rare side effect in a combination vaccine can be difficult. Post-marketing surveillance and clinical trials carefully monitor adverse events, but pinpointing the exact cause is more complex than with single vaccines.

### 5. Scheduling and Coverage Gaps

Fixed antigen combinations can create logistical challenges in vaccination programs. If a child has already received one component, a combination vaccine may result in an unnecessary extra dose. In some cases, a contraindication for one component may prevent the use of the entire vaccine. Additionally, differences in scheduling requirements across brands can create confusion among healthcare providers, necessitating careful record-keeping.

### 6. Regulatory and Testing Complexity

Unlike single vaccines, combination vaccines must demonstrate that each antigen induces an immune response comparable to standalone vaccines. Since full efficacy trials are often impractical, immunogenicity studies measure antibody levels to infer protection. Regulatory approval can be slow and complex, and manufacturers must navigate partnerships and licensing agreements for multiple antigen components, adding to the logistical hurdles in bringing combination vaccines to market.

Monovalent vs. Multivalent Immune Response Effectiveness

A key question is whether combining antigens (either for different diseases or multiple strains) compromises the immune response to any single component. Generally, licensed combination and multivalent vaccines are found to be immunologically equivalent to giving separate shots, meaning they produce protective antibody levels for each component that are on par with those from individual vaccines (Elliman D, et al., 2003 May 10; 326(7397):995-6; Mazunina E P, et al., Vaccines (Basel). 2024 Oct. 24; 12(11):1206)).

Before approval, companies must show that no antigen in the mix unduly weakens the response to another. For example, studies of a 5-in-1 pediatric vaccine (DTaP-IPV-Hib) found no difference in the percentage of infants who reached protective antibody titers for diphtheria, tetanus, pertussis, hepatitis B, and polio when given as a combination versus separately; however, the Hib antibody concentration was somewhat lower in the combo group, though still in an acceptable range. In such cases, manufacturers may increase the antigen dose or add an adjuvant to ensure immunity remains adequate.

In some scenarios, a monovalent vaccine may induce a higher immune response for its single target than a multivalent vaccine does for that same target, simply because the monovalent focuses the immune system on one antigen. A notable example is the oral polio vaccine: studies showed that a monovalent OPV against poliovirus type 1 generated stronger immunity to type 1 polio per dose than the trivalent OPV (which covered types 1, 2, and 3 in one dose) (The Immunogenicity of Monovalent Oral Poliovirus Vaccine Type 1 (mOPV1) and Inactivated Poliovirus Vaccine (IPV) in the EPI Schedule of India—PMC). This higher immunogenicity of monovalent OPV has been leveraged in outbreak settings to rapidly boost immunity to the specific poliovirus type in circulation. That said, the trivalent vaccine's overall benefit was protecting against all three polio types in one course—a trade-off of breadth vs. per-strain strength. In practice, multiple doses of trivalent OPV still produced good immunity to all types, and the convenience and broad coverage outweighed the slightly lower per-dose response. Similarly, bivalent OPV (covering two strains) was shown to be more immunogenic per strain than the trivalent, and it replaced the trivalent OPV in routine use after wild poliovirus type 2 was eradicated (Mohanty L, et al., Vaccines (Basel). 2024 Apr. 17; 12(4):424.).

For multivalent strain vaccines, extensive testing has shown that adding more strains does not inherently weaken the immune response to each strain, as long as the vaccine is well-formulated. The 9-valent HPV vaccine, for instance, was shown in clinical trials to produce antibody responses to HPV types 16 and 18 (the two most important cancer-causing strains) that were non-inferior to those generated by the earlier bivalent or quadrivalent HPV vaccines, while also inducing strong immunity to the five additional strains included (Yang D Y, et al., 2016 May; 62(5):399-402). In other words, protection against the original strains wasn't diluted by adding more types. Likewise, according to the U.S. center for disease control (CDC) sequential versions of pneumococcal conjugate vaccines (PCV) illustrate this: PCV13's immune response for the 7 serotypes it shared with the older PCV7 was comparable to PCV7's response, and it successfully added coverage for six extra serotypes. Field data confirmed that children vaccinated with PCV13 had protection against pneumococcal disease just as good for those original serotypes and gained new protection against the additional serotypes, drastically cutting disease rates for all 13 targets (e.g., review from CDC “About Pneumococcal Vaccines”). These examples underscore that multivalent vaccines can broaden protection without sacrificing effectiveness for each component.

It's worth noting that if any antigen in a combination or multivalent vaccine showed a significantly weaker immune response than desired, it would likely fail to meet licensing requirements. In practice, vaccine formulations are optimized (e.g. by increasing antigen content or adding adjuvants) to ensure each component achieves the needed immunogenicity. Health authorities like the WHO and CDC therefore state that approved combination vaccines are “just as safe and effective” as the individual vaccines (Liu, B. et al., Vaccines 2022, 10, 472). Ongoing surveillance continues to compare real-world effectiveness; so far, no major loss of protection has been found for combination vaccines in use—though, as mentioned, minor differences in antibody levels can occur, they rarely translate into clinical failure (Elliman D, et al., BMJ. 2003 May 10; 326(7397):995-6). In summary, while monovalent vaccines can sometimes maximize the immune response to a single antigen, well-designed multivalent and combination vaccines effectively provide a broad umbrella of immunity, with any small compromises balanced by the significant gains in disease coverage and compliance.

### Case Studies and Real-World Efficacy

### MMR (Measles, Mumps, Rubella) Vaccine

The MMR vaccine combines protection against measles, mumps, and rubella in a single injection, significantly reducing cases of these diseases. Before its introduction, separate vaccines left immunity gaps, leading to continued measles outbreaks and congenital rubella cases. The combined vaccine led to a drastic decline in these diseases. Two doses of MMR provide 97% efficacy against measles and ~88% against mumps, comparable to individual vaccines. Despite past concerns, research from John Hopkins Institute for Vaccine Safety confirmed MMR does not cause autism and is generally safe, with mild side effects such as fever or rash.

### DTP and Pentavalent Vaccines

The DTP vaccine (diphtheria, tetanus, pertussis) has been used since the 1940s and remains essential in disease prevention. Modern versions, like the pentavalent vaccine (DTaP-HepB-IPV/Hib), protect against five diseases in a single shot, increasing vaccine coverage. Studies show that combination vaccines maintain effectiveness, as seen in Korea, where a DTaP-IPV-Hib vaccine was non-inferior to separate doses (Systematic Review and Meta-Analysis, source). The introduction of pentavalent vaccines worldwide has increased uptake of hepatitis B and Hib vaccines, reducing Hib meningitis and liver disease from hepatitis B.

### Pneumococcal Conjugate Vaccines (PCV)

Streptococcus pneumoniae has multiple serotypes, making multivalent vaccines necessary for broad protection. PCV7, licensed in 2000, significantly reduced invasive pneumococcal disease, leading to herd immunity benefits. Later versions, PCV13, PCV15, and PCV20, expanded coverage to additional serotypes, maintaining strong efficacy (CDC, source). PCV13 led to an 84% reduction in invasive disease from newly covered strains, confirming the benefit of increased valency (CDC, source). Safety profiles remained consistent across versions.

### Human Papillomavirus (HPV) Vaccine

Early HPV vaccines targeted four strains, covering ~70% of cervical cancer-causing types. The 9-valent HPV vaccine expanded coverage to ~90% of cancer-causing strains, maintaining 97% efficacy in preventing cervical precancers. Real-world data from vaccinated populations show further declines in HPV-related diseases. The safety profile of the 9-valent vaccine remained comparable to the quadrivalent vaccine, with mild side effects like injection-site reactions.

Polio Vaccines (Monovalent vs. Trivalent OPV)

The oral polio vaccine (OPV) was originally trivalent (tOPV), but research showed that monovalent OPVs (mOPV1, mOPV3) were more effective in targeted eradication efforts. For instance, mOPV1 had significantly higher immunity per dose than tOPV, aiding polio eradication in countries like India. Later, bivalent OPV (bOPV) was introduced to optimize protection against type 1 and type 3 polio while omitting type 2 (which had been eradicated). Despite the advantages of monovalent vaccines in outbreak settings, multivalent vaccines remain crucial for routine immunization programs to ensure broad protection.

### Pharmaceutical Compositions and Formulations.

The disclosed vaccine composition as prepared with the disclosed UVA, UVB, or UVC, including without limitation, far-UVC disinfection system may be formulated as a pharmaceutical composition.

Such a pharmaceutical composition may be in a form suitable for administration to a subject (i.e. mammal), or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In some embodiments, the pharmaceutical compositions useful for practicing the method of the invention may be administered to deliver a dose of between 10⁶ and 10¹² PFU.

In some embodiments, the pharmaceutical compositions useful for practicing the method of the invention may comprise an adjuvant. Non-limiting examples of suitable are Freund's complete adjuvant, Freund's incomplete adjuvant, Quil A, Detox, ISCOMs or squalene.

Pharmaceutical compositions that are useful in the methods of the invention may be suitably developed for inhalation, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically based formulations. The route(s) of administration is readily apparent to the skilled artisan and depends upon any number of factors including the type and severity of the disease being treated, the type and age of the veterinary or human patient being treated, and the like.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modifications with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

The composition of the invention may comprise a preservative from about 0.005% to 2.0% by total weight of the composition. The preservative is used to prevent spoilage in the case of exposure to contaminants in the environment.

### Administration/Dosing

The regimen of administration may affect what constitutes an therapeutically effective amount. For example, the adenovirus vector of the invention may be administered to the subject (i.e. mammal) in a single dose, in several divided dosages, as well as staggered dosages may be administered daily or sequentially, or the dose may be continuously infused, or may be a bolus injection. Further, the dosages may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat the disease in the subject. A therapeutically effective amount of the composition necessary to achieve the intended result will vary and will depend on factors such as the disease to be treated or prevented, the age, sex, weight, condition, general health and prior medical history of the subject being treated, and like factors well-known in the medical arts. In particular embodiments, it is especially advantageous to formulate the composition in dosage unit form for ease of administration and uniformity of dosage. In certain embodiment, dosage unit form as used herein may, for example and without limitation, refer to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the composition and the heterologous protein to be expressed, and the particular therapeutic effect to be achieved.

### Routes of Administration

One skilled in the art will recognize that although more than one route can be used for administration, a particular route can provide a more immediate and more effective reaction than another route. Routes of administration of any of the disclosed compositions of the invention include inhalation, oral, nasal, rectal, parenteral, sublingual, transdermal, epidermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In some embodiment the administration is by epidermal contact. Epidermal contact, as used herein, may, for example, also be referred to as dermal exposure, cutaneous contact, or skin contact. In some embodiment the administration is by epidermal contact.

### Administration Via Skin Contact

Skin contact as a route of administration involves applying substances directly onto the skin surface, allowing for absorption through the epidermis and dermis. Such method leverages the skin's extensive network of capillaries and immune cells to facilitate both local and systemic effects. Skin contact administration is suitable for a variety of therapeutic applications such as vaccines.

In some embodiments, the disclosed vaccination method involves a subject coming into contact with surfaces coated with far-UVC inactivated pathogens. Examples of such surfaces include, but are not limited to, everyday objects such as door handles, countertops, or specially designed medical devices. In some embodiments, the inactivated pathogens on these surfaces are intended to transfer to the skin upon contact, allowing for gradual absorption and interaction with immune cells.

In some embodiments, the surfaces touched or to be touched by a subject, are treated with a stable formulation of far-UVC inactivated pathogens composition that adhere effectively, ensuring prolonged presence and potential immune activation. In some embodiments, the formulation may include binding agents that maintain the pathogens on the surface while allowing for easy transfer to the skin upon touch.

In some embodiments, the surfaces comprising far-UVC inactivated pathogens are integrated into wearable items, such as gloves or wristbands, which are worn by the subject to provide continuous exposure to the inactivated pathogens. In some embodiments, such surfaces provide controlled and sustained immune response over time.

### Administration Via Inhalation

Inhalation as a route of administration involves delivering substances into the respiratory system through the nasal or buccal pathways. Nasal, intranasal instillation, and buccal inhalation provide efficient and non-invasive routes for administering of therapeutic agents such as vaccines.

In some embodiments, the disclosed vaccination method involves a subject inhaling air containing far-UVC inactivated pathogens. In some embodiments, the disclosed far-UVC inactivated pathogens are dispersed into the environment through ventilation systems, ensuring even distribution throughout the room. The inactivated pathogens are designed to be inhaled naturally as the subject breathes, allowing for interaction with the respiratory tract's immune cells.

In some embodiments, the air is treated with a controlled release mechanism that periodically disperses inactivated pathogens, maintaining a consistent concentration in the environment. In some embodiments, the subject receives a steady exposure over time, enhancing the potential for immune activation.

In some embodiments, the disclosed far-UVC inactivated pathogens are integrated into air purification systems that simultaneously clean the air and introduce the far-UVC inactivated pathogens. In some embodiments, such dual-function system ensures that the air remains safe to breathe while providing the intended immunological benefits of exposure to the disclosed far-UVC inactivated pathogens.

In some embodiments, the disclosed far-UVC inactivated pathogens are introduced into the air through humidifiers or diffusers, which create a fine mist that is easily inhaled. In some embodiments, such method allows for efficient delivery of the pathogens to the respiratory system, promoting an effective immune response.

In some embodiments, the environment is equipped with sensors that monitor air quality and pathogen levels, adjusting the release of inactivated pathogens to optimize exposure and immune response. This smart system ensures that the subject receives the appropriate dosage based on real-time environmental conditions.

### Definitions

In certain embodiments, as used herein ultraviolet (UV) light may, for example and without limitation, refer to a form of non-ionizing electromagnetic radiation that comprises wavelengths from approximately 100 nanometers (nm) to 400 nm and is conventionally divided into three subcategories based on wavelength: UV-A, UV-B, and UV-C. UV-A radiation occupies the longest wavelength range, typically from 315 nm to 400 nm. UV-B radiation ranges from 280 nm to 315 nm. UV-C radiation spans from approximately 200 nm to 280 nm; in some embodiments, UVC includes vacuum UV wavelengths down to 100 nm. Far UV-C radiation spans approximately 200 nm to 230 nm; in some embodiments, includes 207-222 nm range.

In certain embodiments, the terms “enhancing immunity” or “enhancing immunogenicity” as used herein may, for example and without limitation, refer to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like.

In certain embodiments, as used herein, the terms “eliciting an immune response” or “immunizing” may, for example and without limitation, refer to the process of generating or activating a T cell or B cell response against a heterologous protein. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physio-chemical process.

In certain embodiments, the term “activated T cell” may, for example and without limitation, refer to a T cell that is currently undergoing cell division, cytokine production, performance of regulatory or cytolytic effector functions, and/or has recently undergone the process of “activation.”

In certain embodiments, the term “antigen” or “Ag” as used herein may, for example and without limitation, refer to a substance or composition capable of eliciting an immune response. This response may include, but is not limited to, the production of antibodies, the activation of immunologically competent cells, or other immune mechanisms. It encompasses a wide array of entities, including but not limited to, molecules, proteins, peptides, viruses, bacteria, and other pathogens. The skilled artisan will recognize that virtually any composition with the potential to interact with the immune system can function as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell, a biological fluid, or a pathogen or a microorganism such as a bacteria or a virus. The term “treatment” as used within the context of the present invention is meant to include therapeutic treatment as well as prophylactic, or suppressive measures for the disease or disorder. In certain embodiments, as used herein, the term “treatment” and associated terms such as “treat” and “treating” may, for example and without limitation, refer to the reduction of the progression, severity and/or duration of a disease condition or at least one symptom thereof. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

An “immunogenic composition” may comprise in a volume of air, a volume of liquid, a volume of vapors, and/or on a surface area, wherein the immunogenic composition is generated by exposing a volume of air, a volume of liquid, a volume of vapors, and/or a surface area to an ultraviolet light (UV) (UVC) disinfection system, such as any UVC disinfection system disclosed herein. The minimum effective concentration of an immunogenic composition capable of eliciting an immune response may be as low as a single antigenic unit. In some embodiments, an antigen may comprise a whole microorganism (e.g., virus, bacterium, fungus, parasite, protozoan or mold), any immunogenic portion thereof (e.g., protein, peptide, nucleic acid fragment, membrane component, or structural fragment) or any other pathogenic particulate matter (e.g., prion or allergen). The immunogenic composition may comprise a pathogen or a plurality of different pathogens that are inactivated, attenuated, weakened, dormant, or killed after exposure to the UV light system as described herein. The immunogenic composition may be monovalent or polyvalent. The immunogenic composition may induce, trigger, enhance, or stimulate an immune response which may include, but is not limited to, generating an antibody against a pathogen or a plurality of pathogens, a humoral immune response, immune priming, inflammation, and IL-8 production. A subject may be exposed to the immunogenic composition. The immunogenic composition may be formulated to suit the route of administration, including but not limited to any route of administration as described herein.

In certain embodiments, as used herein, the term “pharmaceutical composition” may, for example and without limitation, refer to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, intranasal, inhalation, oral, aerosol, parenteral, ophthalmic, pulmonary, administration to a membrane (e.g., a mucus membrane such as the nasal cavity, mouth, or eye) and topical administration.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals or in humans.

“Carriers” as used herein include carriers, excipients, or stabilizers that are nontoxic to the cell or mammal being exposed thereto at the dosages and concentrations employed. Often the carrier is an aqueous pH buffered solution. Examples of carriers include buffers such as phosphate, citrate, and other organic acids; antioxidants including ascorbic acid; low molecular weight (e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant (complete or incomplete)), excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable carriers are described in Remington's Pharmaceutical Sciences (1990) Mack Publishing Co., Easton, PA. Compositions, including pharmaceutical compounds, may contain a prophylactically or therapeutically effective amount of composition (e.g., an immunogenic composition), for example, together with a suitable amount of carrier so as to provide the form for proper administration to the subject (e.g., an animal or human patient). The formulation should suit the mode of administration.

The term “therapeutically effective amount” as used herein refers to the amount of an agent (e.g., immunogenic composition) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction or amelioration of the advancement or progression of a given disease, disorder, or condition, (ii) reduction or amelioration of the recurrence, development or onset of a given disease, disorder or conditions, and/or (iii) to improve or enhance the prophylactic or therapeutic effect of another therapy (e.g., a therapy other than the administration of an immunogenic composition as described herein). A “therapeutically effective amount” of a substance/molecule/agent of the present disclosure (e.g., an immunogenic composition) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In some embodiments, the term “therapeutically effective amount” refers to an amount of an immunogenic composition other agent (e.g., vaccine) effective to “treat” a disease, disorder, or condition, in a subject or mammal.

In certain embodiments, the language “pharmaceutically acceptable carrier” may, for example and without limitation, include a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” may, for example and without limitation, also include any and all coatings, antibacterial and antifungal agents, and absorption delaying agents, and the like that are compatible with the activity of the compound, and are physiologically acceptable to the subject. Supplementary active compounds may also be incorporated into the compositions.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

The following clauses describe certain non-limiting embodiments.

Clause 1: A ultraviolet (UVC) disinfection system comprising: a housing; a light source positioned within the housing and configured to emit a UVC light; a subject detection sensor positioned within the housing and configured to detect the position of a subject and generate corresponding detection data, the subject consisting of one of a human being, a domesticated animal, and a farm animal; and a controller positioned within the housing and in communication with the light source and the subject detection sensor, the controller configured to: receive detection data from the subject detection sensor including position data of the subject; determine, based on the received detection data, whether the subject is within a range of the UVC emitted by the light source; and based on the detection data, adjust an intensity of the UVC light.

Clause 2. The UVC disinfection system of clause 1, wherein the controller is further configured to, based on the position data, determine that a subject is within range of the UVC light and reduce the intensity of the UVC light.

Clause 3. The UVC disinfection system of clause 2, wherein the controller is further configured to, based on the position data, determine that a subject is not within range of the UVC light and increase the intensity of the UVC light.

Clause 4. The UVC disinfection system of clause 1, wherein the controller is further configured to adjust the intensity of the UVC light based on position data of the subject and corresponding timestamps generated by the subject detection sensor.

Clause 5. The UVC disinfection system of clause 1 further comprising: a pathogen detection sensor in communication with the controller and configured to detect the presence of pathogens, and wherein the controller is configured to: receive pathogen detection data from the pathogen detection sensor; and in response to determining that one or more pathogens are detected within the range of the UVC light, cause the intensity of the UVC light emitted by the light source to be increased.

Clause 6. The UVC disinfection system of clause 1, wherein the controller is configured to determine an effective disinfection rate based on an amount of time that the light source has been emitting the UVC light.

Clause 7. The UVC disinfection system of clause 6, wherein the controller is configured to transmit the determined effective disinfection rate to a client device external to the UVC disinfection device.

Clause 8. The UVC disinfection system of clause 1, wherein the subject detection sensor is a plurality of subject detection sensors each in communication with the controller, the plurality of subject detection sensors including two infrared sensors and four motion sensors.

Clause 9. The UVC disinfection system of clause 1, wherein the controller is configured to cause the subject detection sensor to activate at a predetermined detection interval, and wherein, when activated, the subject detection sensor is configured to generate the detection data and transmit the detection data to the processor.

Clause 10. The UVC disinfection system of clause 9, wherein the predetermined detection interval is less than or equal to one second.

Clause 11. The UVC disinfection system of clause 1, wherein the controller is configured to delay causing the light source to emit the UVC light in response to the subject detection sensor detecting no subject within the range of the UVC light emitted by the light source by a predetermined amount of delay time.

Clause 12. The UVC disinfection system of clause 11, wherein the predetermined amount of delay time is between about one second to six minutes.

Clause 13. The UVC disinfection system of clause 1, wherein the controller is further configured to: in response to the light source emitting the UVC light continuously for a predetermined maximum emission amount of time, causing the light source to cease emitting the UVC light.

Clause 14. The UVC disinfection system of clause 13, wherein the predetermined maximum emission amount of time is about sixty minutes.

Clause 15. The UVC disinfection system of clause 1, wherein the light source is configured to emit a UVC light having an output wavelength of about 222 nanometers.

Clause 16. The UVC disinfection system of clause 1, wherein the controller is configured to cause the light source to cease emitting the UVC light in response to the subject detection sensor detecting the presence of a subject within the range of the emitted UVC light for a threshold limit value (TLV) amount of time, wherein the TLV is based on the output wavelength of the emitted UVC light.

Clause 17. The UVC disinfection system of clause 1, wherein the controller is configured to cause the light source to cease emitting the UVC light in response to the subject detection sensor detecting the presence of a subject within a predetermined distance of the light source 104.

Clause 18. The UVC disinfection system of clause 17, wherein the predetermined distance is about three feet.

Clause 19. The UVC disinfection system of any one of clauses 1 to 18, wherein UVC comprises far-UVC.

Clause 20. A UV disinfection system comprising: a light source positioned configured to emit a UV light having an output wavelength of between about 206 nanometers to about 230 nanometers; one or more subject detection sensors configured to detect the presence of one or more subjects, the one or more subjects consisting of one or more of human beings, one or more domesticated animals, and one or more farm animals or a combination thereof, one or more pathogen detection sensors in communication with the controller and configured to detect the presence of pathogens; and a controller in communication with the light source and the one or more subject detection sensors, the controller configured to: receive detection data from the one or more subject detection sensors; determine, based on the received detection data, whether one or more subjects are within a range of the UV light emitted by the light source; in response to determining that a subject of the one or more subjects are within the range of the UV light emitted by the light source, cause an intensity of the UV light emitted by the light source to be decreased; and in response to determining that no subject of the one or more subjects is within the range of the UV light emitted by the light source, cause the intensity of the UV light emitted by the light source to be increased receive pathogen detection data from the one or more pathogen detection sensors; and in response to determining that one or more pathogens are detected within the range of the UV light, cause the intensity of the UV light emitted by the light source to be increased.

Clause 21. A method of automatically disinfecting the air and surfaces within the range of a UV disinfection device, the method comprising: causing a UV disinfection device to emit UVA, UVB, or UVC light, the UV disinfection device including: one or more UV light sources configured to emit UVA, UVB, or UVC light; one or more subject detection sensors configured to detect a position of a subject, the subject consisting of one of a human being, a domesticated animal, and a farm animal; and a controller in communication with the one or more UV light sources and the subject detection sensor and configured to selectively activate and deactivate at least one of the UV light sources or at least one of the one or more subject detection sensors; at the subject detection sensor, detecting the position of the subject and generating detection data therefrom including position data of the subject; transmitting the detection data from the subject detection sensor to the controller, at the controller, adjusting an intensity of at least one of the one or more UV light sources based on the received detection data.

Clause 22. The method of clause 21 further comprising: at the controller, determining, based on the position data, that a subject is within range of the one or more UV light sources and reducing the intensity of the one or more UV light sources.

Clause 23. The method of clause 21 or 22 further comprising: at the controller, determining, based on the position data, that a subject is not within range of the one or more UV light sources and increasing the intensity of the one or more UV light sources.

Clause 24. A method of triggering an immune response in a subject, the method comprising administering to the subject an immunogenic composition comprised in a volume of air and/or on a surface area.

Clause 25. The method of clause 24, further comprising generating the immunogenic composition by exposing the volume of air and/or the surface area to the disinfection system of any one of clauses 1 to 20.

Clause 26. A method of preventing, treating or reducing a disease or a condition in a subject, the method comprising administering to the subject an effective amount of an immunogenic composition prepared by the disinfection system of any one of clauses 1 to 20.

Clause 27. A method for providing anti-pathogen immunity in a subject, the method comprising administering to the subject an effective amount of an immunogenic composition prepared by the disinfection system of any one of clauses 1 to 20.

Clause 28. A method for enhancing immunity in a subject, the method comprising administering to the subject an effective amount of an immunogenic composition prepared by disinfection system of any one of clauses 1 to 20.

Clause 29. A method for stimulating an immune response in a cell population or tissue in a subject, the method comprising administering to the subject an effective amount of an immunogenic composition prepared by the disinfection system of any one of clauses 1 to 20.

Clause 30. The method of any one of clauses 24 to 29, wherein the administering comprises a skin-contact and/or an inhalation.

Clause 31. The method of any one of clauses 24 to 30, wherein the immunogenic composition comprises an inactivated pathogen.

Clause 32. The method of clause 31, wherein the pathogen comprises a virus and/or a bacteria.

Clause 33. The method of any one of clauses 24 to 32, wherein the immunogenic composition is comprised in a volume of air and/or on a surface area.

Clause 34. The method of clause 33, wherein the immunogenic composition is generated by exposing the volume of air and/or the surface area to the disinfection system of any one of clauses 1 to 20.

Clause 35. The method of any one of clauses 24 to 34, wherein the immunogenic composition is comprised in a pharmaceutically acceptable carrier.

Clause 36. The method of any one of clauses 24 to 35, wherein UVC comprises far-UVC.

Clause 37. A pharmaceutical composition for preventing, treating or reducing a disease or a condition in a subject, the pharmaceutical composition comprising an immunogenic composition in a pharmaceutical acceptable carrier, wherein the immunogenic composition is prepared by the UV disinfection system of any one of clauses 1 to 23.

The following clauses describe additional certain non-limiting embodiments.

Clause 1: A method of triggering an immune response in a subject, the method comprising: administering to the subject an immunogenic composition, wherein the immunogenic composition is generated by exposing a volume of air, a volume of liquid, a volume of vapors, and/or a surface area to an ultraviolet light.

Clause 2: A method of triggering an immune response in a subject, the method comprising: exposing a volume of air, a volume of liquid, a volume of vapors, and/or a surface area to an ultraviolet light to generate an immunogenic composition; and administering to the subject an immunogenic composition.

Clause 3: A method for stimulating an immune response in a cell population or tissue in a subject, comprising: administering to the subject an immunogenic composition, wherein the immunogenic composition is generated by exposing a volume of air, a volume of liquid, a volume of vapors, and/or a surface area to an ultraviolet light.

Clause 4: A method of preventing, treating, or reducing a disease or condition in a subject, the method comprising: administering to the subject an immunogenic composition, wherein the immunogenic composition is generated by exposing a volume of air, a volume of liquid, a volume of vapors, and/or a surface area to an ultraviolet light.

Clause 5: A method for providing anti-pathogen immunity in a subject, the method comprising: administering to the subject an immunogenic composition, wherein the immunogenic composition is generated by exposing a volume of air, a volume of liquid, a volume of vapors, and/or a surface area to an ultraviolet light.

Clause 6: A method for enhancing immunity in a subject, the method comprising: administering to the subject an immunogenic composition, wherein the immunogenic composition is generated by exposing a volume of air, a volume of liquid, a volume of vapors, and/or a surface area to an ultraviolet light.

Clause 7: A method of generating an immunogenic composition comprising exposing a volume of air, a volume of liquid, a volume of vapors, and/or a surface area to an ultraviolet light.

Clause 8: The method of any one of clauses 1 to 7, wherein the ultraviolet light comprises UVA, UVB, UVC, or any combination thereof.

Clause 9: The method of any one of clauses 1 to 8, wherein the ultraviolet light comprises UVC.

Clause 10: The method of any one of clauses 1 to 9, wherein the ultraviolet light comprises far-UVC.

Clause 11: The method of any one of clauses 1 to 10, wherein the ultraviolet light comprises an output wavelength of about 254 nm or 254 nm.

Clause 12: The method of any one of clauses 1 to 11, wherein the ultraviolet light comprises an output wavelength of from 315 nm to 400 nm.

Clause 13: The method of any one of clauses 1 to 12, wherein the ultraviolet light comprises an output wavelength of from 280 nm to 315 nm.

Clause 14: The method of any one of clauses 1 to 13, wherein the ultraviolet light comprises an output wavelength of from 100 nm to 280 nm.

Clause 15: The method of any one of clauses 1 to 14, wherein the ultraviolet light comprises an output wavelength of from 200 nm to 280 nm.

Clause 16: The method of any one of clauses 1 to 15, wherein the ultraviolet light comprises an output wavelength of from 230 nm to 280 nm.

Clause 17: The method of any one of clauses 1 to 16, wherein the ultraviolet light comprises an output wavelength of from 200 nm to 230 nm.

Clause 18: The method of any one of clauses 1 to 17, wherein the ultraviolet light comprises an output wavelength of from 206 nm to 230 nm.

Clause 19: The method of any one of clauses 1 to 18, wherein the ultraviolet light comprises an output wavelength of about 222 nm or 222 nm.

Clause 20: The method of any one of clauses 1 to 19, wherein the ultraviolet light is emitted from an ultraviolet (UVC) disinfection system.

Clause 21: The method of any one of clauses 1 to 20, wherein the volume of air comprises a suspended liquid particle.

Clause 22: The method of any one of clauses 1 to 21, wherein the volume of air comprises a respiratory droplet.

Clause 23: The method of any one of clauses 1 to 22, wherein the volume of air, volume of liquid, volume of vapors, and/or surface area comprises a particulate matter.

Clause 24: The method of any one of clauses 1 to 23, wherein the particulate matter comprises a liquid particle.

Clause 25: The method of any one of clauses 1 to 24, wherein the particulate matter comprises a respiratory droplet.

Clause 26: The method of any one of clauses 1 to 25, wherein the volume of air, volume of liquid, volume of vapors, and/or surface area comprises a pathogen.

Clause 27: The method of any one of clauses 1 to 26, wherein the volume of air, volume of liquid, volume of vapors, and/or surface area comprises a plurality of different pathogens.

Clause 28: The method of any one of clauses 26 to 27, wherein the pathogen or the plurality of different pathogens are aerosolized.

Clause 29: The method of any one of clauses 26 to 28, wherein the pathogen is a microbe.

Clause 30: The method of any one of clauses 26 to 29, wherein the pathogen is a bacteria, virus, mold, parasite, allergen, fungi, protozoa, or prion.

Clause 31: The method of any one of clauses 27 to 30, wherein the plurality of different pathogens comprises two or more of a group consisting of a bacteria, virus, mold, parasite, allergen, fungi, protozoa, and prion.

Clause 32: The method of any one of clauses 27 to 31, wherein the plurality of different pathogens comprises two or more different types or species of a bacteria, a virus, mold, parasite, allergen, a fungi, a protozoa or a prion.

Clause 33: The method of any one of clauses 1 to 32, wherein the immunogenic composition comprises an uncharacterized pathogen or uncharacterized plurality of different pathogens.

Clause 34: The method of any one of clauses 26 to 33, wherein the pathogen is inactivated, attenuated, weakened, dormant, or killed after exposure to the ultraviolet light.

Clause 35: The method of clause 34, wherein the pathogen is inactivated, attenuated, weakened, dormant, or killed pathogen or a particle thereof.

Clause 36: The method of clause 34 or 35, wherein the inactivated, attenuated, weakened, dormant, or killed pathogen or a particle thereof comprises one or more epitopes for generating an immune response; optionally, wherein the immune response is against the pathogen.

Clause 37: The method of any one of clauses 34 to 36, wherein the inactivated, attenuated, weakened, dormant, or killed pathogen or particle thereof is an aerosolized inactivated pathogen.

Clause 38: The method of any one of clauses 34 to 37, wherein the dormant pathogen is viable but not infectious or not culturable.

Clause 39: The method of any one of clauses 1 to 38, wherein the volume of air, volume of liquid, volume of vapors, and/or surface area comprises at least from 10⁸ to 10¹¹ inactivated, attenuated, weakened, dormant, or killed virus or particles thereof.

Clause 40: The method of any one of clauses 1 to 39, wherein the volume of air, volume of liquid, volume of vapors, and/or surface area comprises at least 5×10¹⁰ inactivated, attenuated, weakened, dormant, or killed virus or particles thereof.

Clause 41: The method of any one of clauses 1 to 39, wherein the volume of air, volume of liquid, volume of vapors, and/or surface area comprises from 10⁸ to 10¹¹ inactivated, attenuated, weakened, dormant, or killed virus or particles thereof.

Clause 42: The method of one of clauses 1 to 41, wherein the volume of air, volume of liquid, volume of vapors, and/or surface area comprises 5×10¹⁰ inactivated, attenuated, weakened, dormant, or killed virus or particles thereof.

Clause 43: The method of any one of clauses 1 to 6 or clauses 8 to 42, wherein administering comprises contacting to skin, via inhalation, via intranasal, contacting to a membrane, or any combination thereof.

Clause 44: The method of clause 43, wherein the membrane is a mucus membrane.

Clause 45: The method of any one of clauses 1 to 6 or clauses 8 to 44, wherein the membrane is in the nasal cavity, mouth, or eye.

Clause 46: The method of any one of clauses 1 to 6 or clauses 8 to 45, wherein intranasal is via intranasal instillation.

Clause 47: The method of any one of clauses 1 to 6 or clauses 8 to 46, wherein the subject comprises an immune system.

Clause 48: The method of any one of clauses 1 to 6 or clauses 8 to 47, wherein the subject is an animal.

Clause 49: The method of any one of clauses 1 to 6 or clauses 8 to 48, wherein the subject is a mammal, a reptile, an amphibian, or a fish.

Clause 50: The method of any one of clauses 1 to 6 or clauses 8 to 49, wherein the subject is an undomesticated animal.

Clause 51: The method of one of clauses 1 to 6 or clauses 8 to 49, wherein the subject is a domesticated animal.

Clause 52: The method of any one of clauses 1 to 6 or clauses 8 to 49, wherein the subject is human.

Clause 53: The method of any one of clauses 1 to 3 or clauses 8 to 52, wherein the immune response comprises generating an antibody against a pathogen.

Clause 54: The method of any one of clauses 1 to 3 or clauses 8 to 53, wherein the immune response is a humoral immune response.

Clause 55: The method of any one of clauses 1 to 3 or clauses 8 to 54, wherein the immune response is immune priming.

Clause 56: The method of any one of clauses 1 to 3 or clauses 8 to 55, wherein the immune response comprises inflammation.

Clause 57: The method of one of clauses 1 to 3 or clauses 8 to 56, wherein the immune response comprises IL-8 production.

Clause 58: The method of any one of clause 4 or clauses 8 to 57, wherein the disease or condition is associated with or resulting from a microbe.

Clause 59: The method of any one of clause 4 or clauses 8 to 58, wherein the disease or condition is associated with or resulting from a bacteria, virus, mold, allergen, parasite, allergen, fungi, protozoa, and prion.

Clause 60: The method of any one of clause 4 or clauses 8 to 59, wherein the disease or condition is RSV, influenza, or coronavirus.

Clause 61: The method of any one of clause 3 or clauses 8 to 60, wherein the cell population comprises an immune cell.

Clause 62: The method of any one of clause 3 or clauses 8 to 61, wherein the cell population comprises a cell comprising a major histocompatibility complex.

Clause 63: The method of any one of clause 3 or clauses 8 to 62, wherein the cell population comprises an antigen presenting cell.

Clause 64: The method of any one of clause 3 or clauses 8 to 63, wherein the cell population comprises a dendritic cell, macrophage, B-cell, T-cell, NK cell, NKT cell, mast cell, basophil, granulocyte, innate lymphoid cell, endothelial cell, fibroblast, or any combination thereof.

Clause 65: The method of any one of clause 3 or clauses 8 to 64, wherein the tissue is an organ, blood, muscle, mucus membrane, or any combination thereof.

Clause 66: The method of any one of clauses 1 to 65, wherein the ultraviolet light is emitted from an ultraviolet light system comprising a light source.

Clause 67: The method of clause 66 wherein the ultraviolet light system comprises: a) a housing; and b) a light source that emits the ultraviolet light.

Clause 68: The method of clause 66 or 67, wherein the ultraviolet light system comprises a trigger subject detection sensor.

Clause 69: The method of clause 68, wherein the trigger subject detection sensor is positioned within the housing.

Clause 70: The method of clause 68 or 69, wherein the trigger subject detection sensor is configured to detect the position of a subject.

Clause 71: The method of any one of clauses 68 to 70, wherein the trigger subject detection sensor is configured to generate detection data on the position of a subject.

Clause 72: The method of any one of clauses 68 to 71, wherein a controller is configured to receive detection data from the trigger subject including position data of the subject, determine, based on the received detection data, whether the trigger subject is within a range of the ultraviolet light emitted by the light source, and based on the detection data, adjust an intensity of the ultraviolet light.

Clause 73: The method of clause 72, wherein the controller is further configured to, based on the position data, determine that a trigger subject is within range of the ultraviolet light and reduce the intensity of the ultraviolet light.

Clause 74: The method of clause 71 or 72, wherein the controller is further configured to, based on the position data, determine that a trigger subject is not within range of the ultraviolet light and increase the intensity of the ultraviolet light.

Clause 75: The method of any one of clauses 71 to 74, wherein the controller is further configured to adjust the intensity of the ultraviolet light based on position data of the trigger subject and corresponding timestamps generated by the subject detection sensor.

Clause 76: The method of any one of clauses 66 to 75, wherein the ultraviolet light system further comprises a pathogen detection sensor.

Clause 77: The method of any one of clauses 66 to 76, wherein the ultraviolet light system further comprises: a pathogen detection sensor in communication with the controller and configured to detect the presence of pathogens.

Clause 78: The method of any one of clauses 72 to 77, wherein the controller is configured to: receive pathogen detection data from the pathogen detection sensor; and in response to determining that one or more pathogens are detected within the range of the ultraviolet light, cause the intensity of the ultraviolet light emitted by the light source to be increased.

Clause 79: The method of any one of clauses 72 to 78, wherein the controller is configured to determine an effective disinfection rate based on an amount of time that the light source has been emitting the ultraviolet light.

Clause 80: The method of any one of clauses 72 to 79, wherein the controller is configured to determine an effective pathogen inactivation rate based on an amount of time that the light source has been emitting the ultraviolet light.

Clause 81: The method of any one of clauses 72 to 80, wherein the controller is configured to transmit the determined effective disinfection rate and/or the determined effective pathogen inactivation rate to a client device external to the ultraviolet light system.

Clause 82: The method of any one of clauses 72 to 81, wherein the trigger subject detection sensor is a plurality of trigger subject detection sensors in communication with the controller.

Clause 83: The method of clause 82, wherein the plurality of trigger subject detection sensors comprises two infrared sensors and four motion sensors.

Clause 84: The method of any one of clauses 72 to 73, wherein the controller is configured to cause the trigger subject detection sensor to activate at a predetermined detection interval, and wherein, when activated, the trigger subject detection sensor is configured to generate the detection data and transmit the detection data to a processor.

Clause 85: The method of any one of clauses 72 to 74, wherein the controller is further configured to: in response to the light source emitting the ultraviolet light continuously for a predetermined maximum emission amount of time, causing the light source to cease emitting the ultraviolet light.

Clause 86: The method of any one of clauses 72 to 75, wherein the controller is configured to cause the light source to cease emitting the ultraviolet light in response to the trigger subject detection sensor detecting the presence of a trigger subject within the range or within a predetermined distance of the emitted ultraviolet light for a threshold limit value (TLV) amount of time, wherein the TLV is based on an output wavelength of the emitted ultraviolet light.

Clause 87: The method of any one of clauses 1 to 6 or clauses 8 to 86, wherein the subject is a trigger subject.

Clause 88: The method of any one of clauses 66 to 87, wherein the ultraviolet light system is a UVA system, UVB system, UVC system, or any combination thereof.

Clause 89: The method of any one of clauses 66 to 88, wherein the ultraviolet light system emits an output wavelength of from 315 nm to 400 nm.

Clause 90: The method of any one of clauses 66 to 89, wherein the ultraviolet light system emits an output wavelength of from 280 nm to 315 nm.

Clause 91: The method of any one of clauses 66 to 90, wherein the ultraviolet light system emits an output of from 100 nm to 280 nm.

Clause 92: The method of any one of clauses 66 to 91, wherein the ultraviolet light system emits an output of from 230 nm to 280 nm.

Clause 93: The method of any one of clauses 66 to 92, wherein the ultraviolet light system emits an output of from 200 nm to 230 nm.

Clause 94: The method of any one of clauses 66 to 93, wherein the ultraviolet light system emits an output of from 206 nm to 230 nm.

Clause 95: The method of any one of clauses 66 to 94, wherein the ultraviolet light system emits an output of about 254 nm or 254 nm.

Clause 96: The method of any one of clauses 66 to 95, wherein the ultraviolet light system emits an output of about 222 nm or 222 nm.

Clause 97: The method of any one of clauses 66 to 96, wherein the ultraviolet light system is a UVC system; optionally, wherein the UVC system is a UVC disinfecting system.

Clause 98: The method of any one of clauses 66 to 97, wherein the ultraviolet light system is a far-UVC system.

Clause 99: The method of any one of clauses 66 to 98, wherein the ultraviolet light system is a 254 nm system that emits an output wavelength of about 254 nm or 254 nm.

Clause 100: The method of any one of clauses 66 to 99, wherein the ultraviolet light system is a 222 nm system that emits an output wavelength of about 222 nm or 222 nm.

Clause 101: An immunogenic composition produced by the process of exposing a volume of air, a volume of liquid, a volume of vapors, and/or a surface area to an ultraviolet light.

Clause 102: The immunogenic composition of clause 101, wherein the ultraviolet light comprises UVA, UVB, UVC, or any combination thereof.

Clause 103: The immunogenic composition of clauses 101 or 102, wherein the ultraviolet light comprises UVC.

Clause 104: The immunogenic composition of any one of clauses 101 to 103, wherein the ultraviolet light comprises far-UVC.

Clause 105: The immunogenic composition of any one of clauses 101 to 104, wherein the ultraviolet light comprises an output wavelength of from 315 nm to 400 nm.

Clause 106: The immunogenic composition of any one of clauses 101 to 105, wherein the ultraviolet light comprises an output wavelength of from 280 nm to 315 nm.

Clause 107: The immunogenic composition of any one of clauses 101 to 106, wherein the ultraviolet light comprises an output wavelength of from 100 nm to 280 nm.

Clause 108: The immunogenic composition of any one of clauses 101 to 107, wherein the ultraviolet light comprises an output wavelength of from 230 nm to 280 nm.

Clause 109: The immunogenic composition of any one of clauses 101 to 108, wherein the ultraviolet light comprises an output wavelength of from 200 nm to 230 nm.

Clause 110: The immunogenic composition of any one of clauses 101 to 109, wherein the ultraviolet light comprises an output wavelength of from 206 nm to 230 nm.

Clause 111: The immunogenic composition of any one of clauses 101 to 110, wherein the ultraviolet light comprises an output wavelength of about 254 nm or 254 nm.

Clause 112: The immunogenic composition of any one of clauses 101 to 111, wherein the ultraviolet light comprises an output wavelength of about 222 nm or 222 nm.

Clause 113: The immunogenic composition of any one of clauses 101 to 112, wherein the ultraviolet light is emitted from an ultraviolet (UVC) disinfection system.

Clause 114: The immunogenic composition of any one of clauses 101 to 113, wherein the volume of air comprises a suspended liquid particle.

Clause 115: The immunogenic composition of any one of clauses 101 to 114, wherein the volume of air comprises a respiratory droplet.

Clause 116: The immunogenic composition of any one of clauses 101 to 115, wherein the volume of air, volume of liquid, volume of vapors, and/or surface area comprises a particulate matter.

Clause 117: The immunogenic composition of clause 116, wherein the particulate matter comprises a liquid particle.

Clause 118: The immunogenic composition of clause 116 or 117, wherein the particulate matter comprises a respiratory droplet.

Clause 119: The immunogenic composition of any one of clauses 101 to 118, wherein the volume of air, volume of liquid, volume of vapors, and/or surface area comprises a pathogen.

Clause 120: The immunogenic composition of any one of clauses 101 to 119, wherein the volume of air, volume of liquid, volume of vapors, and/or surface area comprises a plurality of different pathogens.

Clause 121: The immunogenic composition of clause 119 or 120, wherein the pathogen or the plurality of different pathogens are aerosolized.

Clause 122: The immunogenic composition of any one of clauses 119 to 121, wherein the pathogen is a microbe.

Clause 123: The immunogenic composition of any one of clauses 119 to 122, wherein the pathogen is a bacteria, virus, mold, parasite, allergen, fungi, protozoa, or prion.

Clause 124: The immunogenic composition of any one of clauses 120 to 123, wherein the plurality of different pathogens comprises two or more of a group consisting of a bacteria, virus, mold, parasite, allergen, fungi, protozoa, and prion.

Clause 125: The immunogenic composition of any one of clauses 120 to 124, wherein the plurality of different pathogens comprises two or more different types or species of a bacteria, a virus, mold, parasite, allergen, a fungi, a protozoa or a prion.

Clause 126: The immunogenic composition of any one of clauses 101 to 125, wherein the immunogenic composition comprises an uncharacterized pathogen or uncharacterized plurality of different pathogens.

Clause 127: The immunogenic composition of any one of clauses 119 to 126, wherein the pathogen is inactivated, attenuated, weakened, dormant, or killed after exposure to the ultraviolet light.

Clause 128: The immunogenic composition of any one of clauses 119 to 126, wherein the pathogen is inactivated, attenuated, weakened, dormant, or killed pathogen or a particle thereof.

Clause 129: The immunogenic composition of any clause 127 or 128, wherein the inactivated, attenuated, weakened, dormant, or killed pathogen or a particle thereof comprises one or more epitopes for generating an immune response; optionally, wherein the immune response is against the pathogen.

Clause 130: The immunogenic composition of any one of clauses 127 to 129, wherein the inactivated, attenuated, weakened, dormant, or killed pathogen or particle thereof is an aerosolized inactivated pathogen.

Clause 131: The immunogenic composition of any one of clauses 127 to 130, wherein the dormant pathogen is viable but not infectious or not culturable.

Clause 132: The immunogenic composition of any one of clauses 101 to 131, wherein the volume of air, volume of liquid, volume of vapors, and/or surface area comprises at least from 10⁸ to 10¹¹ inactivated, attenuated, weakened, dormant, or killed virus or particles thereof.

Clause 133: The immunogenic composition of any one of clauses 101 to 132, wherein the volume of air, volume of liquid, volume of vapors, and/or surface area comprises at least 5×10¹⁰ inactivated, attenuated, weakened, dormant, or killed virus or particles thereof.

Clause 134: The immunogenic composition of any one of clauses 101 to 133 comprising at least from 10⁸ to 10¹¹ inactivated, attenuated, weakened, dormant, or killed virus or particles thereof.

Clause 135: The immunogenic composition of any one of clauses 101 to 134 comprising at least 5×10¹⁰ inactivated, attenuated, weakened, dormant, or killed virus or particles thereof.

Clause 136: The immunogenic composition of any one of clauses 101 to 135 comprising from 10⁸ to 10¹¹ inactivated, attenuated, weakened, dormant, or killed virus or particles thereof.

Clause 137: The immunogenic composition of any one of clauses 101 to 136 comprising 5×10¹⁰ inactivated, attenuated, weakened, dormant, or killed virus or particles thereof.

Clause 138: A pharmaceutical immunogenic composition comprising: a) the immunogenic composition of any one of clauses 101-137; and b) a pharmaceutically acceptable carrier and/or excipient.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order. When specifying a numerical value or range of values, the term “about” means +/−10% unless otherwise defined.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

### EXAMPLES

### Example 1: Immune Response Induction by Far-UVC Inactivated Airborne Pathogens

Far-1. Current Standard UV Inactivation have Numerous Limitations

A 2010 study (Oshansky C M, et al., J Infect Dis. 2010 Apr. 15; 201(8):1201-7) found that UV-inactivated RSV showed that treatment of NHBE cells induces apical IL-8, IP-10, and MCP-1 secretion independent of infection suggesting that RSV proteins alone modify the chemokine response pattern which may affect the early immune response prior to infection. The results of this study demonstrated that without viral replication, the proteins associated with a pathogen can modulate immune responses.

This finding was further corroborated in a study by Rezaee and colleagues (Rezaee F, et al., Am J Respir Cell Mol Biol. 2011 August; 45(2):277-86) which concluded that while less than full activation, exposure to UV-inactivated RSV still induced cytokine expression. The Rezaee findings indicated that inactivation of the viral nucleic acid by irradiation with UV light reduces significantly the effect of RSV on cytokine expression in BMSCs, implying that this effect is primarily linked to the expression and replication of the viral genome. However, for most cytokines, a significant component of this effect can still be detected after inactivation of the viral nucleic acid and thus seems to be associated with a direct toxic effect of the capsid proteins. This observation is consistent with a number of previous studies, including a recent report showing minimal differences in direct comparisons between the proteomes from airway epithelial cells exposed to live or UV-inactivated RSV. More specifically, in lungs and in circulating leukocytes, UV-inactivated RSV still induces cytokines, such as IL-6 and IL-8, and chemokines, such as CCL6, CCL11, CCL22, and CXCL5, and inhibits lymphocyte responses.

Despite these findings, there are significant deficiencies in the aforementioned investigations such as the following:
Did not utilize far-UVC: The lack of application of far-UVC to these studies is a material and significant gap given the potential for its use around people, which requires further research and analysis. The experiments must be completed with far-UVC for applicability. Note: The data is generated using standard medical grade UV and is confirmed as 254 nm in at least one paper.
Only utilize one virus: In addition, the studies only utilize one virus: RSV. This limits the findings and conclusion as viruses vary greatly in attributes like size, replication, transmission, genetic material, protein coat, etc. Application will be necessary to multiple standard viruses and may further apply to bacteria and/or molds.
No Bacteria, Molds: The studies do not look at other pathogens that may incite an immune response, including bacteria and/or molds
Concurrent Exposure: In addition, studies will need to be done on concurrent exposure to multiple pathogens (viruses, bacteria, molds, or other) as this is likely the type of exposure people will encounter.
Partial Responses: The analyses above are also limited in scope to certain measurements and parts of the immune system, which may allow for improper conclusions.
In Vitro vs. In Vivo: Lastly, the studies were limited to in vitro studies with partial and do not properly measure real-world settings and the setup, measurements, and application to an environment in which there are one more individuals exposed to one or more pathogens.
Proper Exposure Levels: There is no optimization or testing done on the proper amount of inactivated pathogen in order to provoke an immune response, only limited tests (the tests are looking for immune responses in the body to RSV and using the UV as a control variable, rather than using the UVC itself to inactivate various levels and concentrations of pathogens).
Age: These tests were primarily completed with infants/young children; age is an important contributor to immune system health and status and must be included in any further studies.
Medium/Substrate: The tests do not include data on any environmental scenarios, including surface-based pathogens that can be swept up into the air when disturbed.
Application of UV light, including the more recently utilized far-UVC light at 222 nm to live pathogens, ranging from viruses, to molds, to bacteria, inactivating them resulting in airborne environment comprising inactivated pathogens. The frequency of light offers effective disinfection while being safe for occupied spaces.

As previously noted, there are significant limitations of existing research, leading to a material gap. The studies did not utilize the proper frequency, focused only on one virus (no bacteria, molds, or other viruses), did not measure concurrent exposure to multiple pathogens, were done only in the lab at pre-set, non-optimized levels, and only measured parts of the immune system. All of these variables need to be reviewed and optimized.

### 2. The Disclosed Far-UVC Inactivation has Many Benefits

Investigations considered: (1) analysis of aerosolized/inhaled vaccines to demonstrate that exposure to inactivated pathogens via aerosolized delivery can generate robust immune responses in a target individual; (2) analysis of subinfectious doses to show their capability to confer some forms of immunity; (3) analysis of the efficacy and safety of multivalent and combination vaccines to show the ability of the human body to respond to many antigens at one time; (4) investigations of locations in the body that produce immune responses based on UV-inactivated viruses; and (5) determination of the optimal immune system response measurement, and the impact of using far-UVC specifically.

The present disclosure demonstrates that breathing in environmental airborne pathogens inactivated by far-UVC at a variety of inactivation levels confer an immune response of some form in one or more places in the human body, across one or more pathogens (viruses, bacteria, and molds), at a variety of infectiveness levels (whether subinfectious or higher).

### Initial Study

A study is conducted to evaluate the effects of the disclosed far-UVC system and to evaluate the effects of the far-UVC inactivated viruses (RSV, Rhinovirus, and Influenza A) on cytokine induction in human nose organoid (HNO) models.

The use of the disclosed far-UVC system results in inactivated virus that provokes an immune response in a human-nose organoid.

### Subsequent Studies

Follow-up tests utilizing far-UVC are conducted based on the below criteria and measures:
1. Measuring the level of immune response from the human-nose organoid based on exposure to far-UVC inactivated pathogens (virus, bacteria, other, or allergens if applicable).
2. Measuring the levels of immune responses from the far-UVC-inactivated pathogens (virus, bacteria, other, or allergens if applicable) in various locations in the body, i.e. infectious or subinfectious dosages.
3. Measuring the immune impact of multiple concurrent viruses on different portions of the immune system.
4. Measuring the immune impact of multiple concurrent pathogens (viruses, bacteria, molds) on different portions of the immune system.
5. Measuring the immune impact for each of the above at various parts of the immune system in a the human body
6. Looking at the impact of In Vivo usage of the far-UVC device on the immune system of individuals exposed to a variety of inactivated pathogens at a variety of levels.
7. Measuring and optimizing the benefits of the Rimoldi Effect with the presently disclosed far-UVC device.
8. Measuring the impact of the technology based on the parameters above for individuals at different ages, gender, and other personal attributes
9. The impact of surface-based pathogens or other pathogens in substrates not in the air that are subsequently disturbed and ingested by an individual according to the levels and parameters above.

### Optimizing the Operation of the Disclosed Far-UVC Device

As a result of these tests, additional research is conducted. Measurements and calculations are computed to assess how the energy (intensity) of the far-UVC device's output is modulated to increase or decrease an immune response. Optimal disinfection and corresponding immune responses are achieved based on the combination and modulation of various factors such as the below:

### Device Operations

1. Adjusting the intensity of a single device through a variety of methods, including on/off or attenuating or increasing intensity;
2. Adding additional devices and operating said devices through a variety of methods, including on/off or attenuating or increasing intensity of said devices; and/or
3. The amount of time a single far-UVC device or multiple far-UVC devices utilized.

### Physical Space

1. The layout or details of the physical space;
2. The location of the pathogens;
3. The location of individuals in the room;
4. The number of individuals in a given room;
5. Detected Pathogens (live);
6. The types of pathogens in the air;
7. The volume or concentration measured for each pathogen in the air;
8. The size, strength, virulence, and R-naught rate of each pathogen detected;
9. Analysis of Public Health Data for Pathogen Presence;
10. The size, strength, virulence, and R-naught rate of each pathogen detected; and/or
11. The relevant volume or concentration of the pathogen's transmission in a given geographic limit (city, state, federal, etc.).

### Example 2: Real-Time Feedback Loop and Optimization and Associated Operations

The clear and intended purpose is to optimize the product for maximum immune response by any individuals in the room.

In addition to the optimization above, The far-UVC product and its corresponding devices (e.g., a computer, or a processor). and features continually collect data, including but not limited to the following:
Energy output and device functionality
The characteristics of a room in which far-UVC is operating, including layout, airflow, or other physical attributes of the space in which the device functions
The presence of, location of, and number of individuals in a given room
Relevant Public health data about pathogen outbreaks in a given geographic limit
The presence, type, location, volume, and attributes of any pathogens in the room.
Any physical characteristics, attributes, or details of the individuals in the room

Based on these parameters, the device's automated algorithm will have the power to:
1. Automatically calculate, in real-time, the optimal usage of the product to maximize the immunity for a single individual or a group of individuals in any given room.
2. Automatically adjust the output level of a single device or multiple far-UVC devices to maximize the immunological effects of the far-UVC inactivated viruses on any individuals

### Applications

Examples of applications and approaches considered in the present disclosure include the following:
Use head and neck squamous cell carcinoma (HNSCC) cell lines such as HNO210 or HNO97.
Testing of the disclosed far-UVC system to provide UV inactivation, check for residual live virus.
Infecting with live viruses or treating with UV inactivated viruses and collecting samples over the next four days.
Once all samples are collected, confirming that the UV inactivated treated trans-wells did not grow any infectious or live virus.
Testing basolateral samples for cytokines and antiviral response
Quality checking all the data and entering into database for analysis.

### Variations

1. In addition to an automated algorithm, information may be presented to an operator of the device based on the attributes above, at which point an individual may manually adjust the settings to alter the impact of the immune response. Said interactions may be done via web applications, phone applications, voice commands, or other methods based on altering the settings of the device.
2. In addition to far-UVC (200-222 nm), and where relevant, use traditional or other forms of UV light (222 nm-300 nm, UVA, UVB, etc.) or others form of electromagnetic radiation can be considered on the EM spectrum to:
   Optimize the operation of a device emitting said energy
   Calculate, in real-time, immune-boosting benefits associated with the operation of said device where it may manually or automatically update to the benefit of individuals located in a given physical space in which said technology is used.
   Automatically or manually calculate other health benefits of the use or operation of any of the above frequencies of light or energy on the EM spectrum, including far-UVC (200-222 nm), 222 nm-300 nm, UVA, UVB, or other forms of electromagnetic radiation.
3. In addition to the health benefits associated with inactivated pathogens boosting immunity, the technology may be used in the same manner, whether manually or automatic, in any of the noted spectrums of use under section 2, for any other health benefits found other than optimizing immunity.

### Example 3: In Vivo Vaccination Based on Far-UVC Inactivation Protocol Triggers Immune System Response and is Safe and Effective

An in vivo study is conducted in BALB/c mice in order to evaluate the effects of virus inactivation (specifically influenza virus) using the presently disclosed protocol using the far-UVC inactivation system. The study aims to assess the protocol's effectiveness by comparing groups exposed to standard viral challenge, partially inactivated virus (50%-75%), and fully inactivated virus (100%), using endpoints such as morbidity/mortality, body weight, health checks, viral load in lungs, and the results of blood and organ analysis. The clinical in vivo phase of the study is for about seven days, and it includes detailed procedures for virus challenge, sample collection, and subsequent analysis. The related materials and methods are described below herein.

### Materials and Methods

### Animal Model and Housing

Nine female BALB/c mice, aged 6-8 weeks, were used in the study. The mice were housed three per cage and acclimated during a 5-7-day pretreatment period prior to any procedures. Standard health checks and body weight measurements were conducted daily for Groups 1 and 2. Animals were continuously monitored for morbidity and mortality.

### Study Design and Groups

The study comprised three groups, with three mice per group (total N=9), designed around a standard viral challenge and subsequent endpoint assessments. All groups received a pre-bleed on Day −1 and were challenged with virus on Day 0.

### Virus and Inactivation Protocol

The challenge virus used was either Influenza A/TX/36/91 or Influenza A/PR/8/34, administered intranasally at a dose of LD90 in 35 μL per mouse. The total number of challenges was nine, distributed among the groups. For Groups 2 and 3, the virus was inactivated using the disclosed far-UVC inactivation system. A back-titer for a “dry run” to review the inactivation protocol was conducted by plaque assay prior to initiation of the in-life phase; this included four conditions (0% inactivation, 100% inactivation, and two conditions for partial inactivation at 50-75%)

Challenge dose vials were stored at −80° C. after administration.

### Challenge Regimen

On Day 0, all groups were administered viral challenge as described above. The route of administration was intranasal. A total of three groups received the challenge. All administration was performed as a single event, totaling nine individual intranasal challenges.

### Blood Collection (Bleeds)

Retro-orbital (RO) bleeds were performed throughout the experiment. Pre-bleeds occurred on Day −1 and test bleeds on Day 3 for all groups. Terminal bleeds were scheduled for Day 6, with the route being either retro-orbital or cardiac puncture. Each mouse had one aliquot collected per event. Altogether, there were 27 blood tubes used, covering all collection events for each animal.

Serum or plasma was isolated from all blood samples. Samples were stored at −80° C. If downstream Luminex testing was to be performed, serum samples would be transferred to the appropriate group.

### Organ Harvest and Processing

Terminal lung harvests were performed for all animals, yielding nine lung samples in total. Each lung was divided into two aliquots. One aliquot per lung was stored at −80° C. and reserved for potential Luminex testing. The second aliquot was homogenized and prepared for viral load determination. Viral load was assessed using plaque assay at a single timepoint (Day 6) and included all nine lung homogenates (three per group)

### Analytical Methods

Viral Load Quantification: Viral load in lung homogenates was determined by standard plaque assay.

Luminex Panel (Conditional): Select samples would undergo Luminex multiplex cytokine analysis. The Luminex panel to be used was to be determined (TBD). Lung homogenates (nine samples: one per animal) and serum (up to 27 samples: one per animal per timepoint, up to three timepoints) would be analyzed.

### Data Collection and Storage

Raw data, including animal health scores, body weights, and other observations, were to be recorded. Serum and lung aliquots for analysis or further testing were stored at −80° C. All procedures adhered to non-GLP standards.

### Endpoint Assessments and Scheduling

Clinical Monitoring: Daily for health and weight in Groups 1 and 2.

Pre-Bleed: Day −1 (all groups).

Challenge: Day 0 (all groups).

Test Bleed: Day 3 (all groups).

Terminal Organ Harvest and Bleed: Day 6 or day of death.

Timeline: The full in-life portion of the study spanned 7 days.

Sample Handling: All biological samples were appropriately aliquoted, stored at −80° C., and processed according to the procedures noted above.

### Special Procedures and Controls

Challenge dose back-titer verification and dry run for the inactivation protocol were incorporated for quality control prior to live animal testing.

### Results:

The result show that virus inactivated with the disclosed far-UVC inactivation device and related protocol triggers immune system response and provides a safe and effective vaccination method.

### Example 4: Influenza Inactivation and Immune Response Study

A study is performed to evaluate the immune response in mice when challenged with influenza virus inactivated with the present Far UVC device. The goal is to compare immune responses elicited by partially and fully inactivated virus to that of live virus, assessing both protection and cytokine activation patterns.

### Experimental Design Overview

The study involves different groups of female BALB/c mice aged 6-8 weeks.
Group 1 is the control group with 0% inactivation (live virus) and consists of 3 mice.
Group 2 involves partial inactivation of 50-75%, either drawn at half-time or mixed 1:1 (live:inactivated).
Group 3 involves full inactivation at 100%.

The virus strain used is Influenza A/PR/8/34, with the challenge route being intranasal (IN) The challenge dose is LD90, 35 uL per animal, and the housing is 3 mice per cage.

### 1—Viral Inactivation and Plaque Assay (Pre-Challenge)

The Beacon Light device is positioned 1 ft above open tubes inside a biosafety hood.

Exposure conditions include 0 min (control), 8 minutes for 50% inactivation, and 50 minutes for 99% inactivation time. Samples are pipetted at 4 minutes for the 8-minute 50% inactivation sample and at 15, 30, and 45 minutes for the 50-minute 99% inactivation (details are listed in Table 1 provided below herein).

Kill Curve with Far UVC Device

The kill curve for the present far UVC device is set up at 12.48 uW/cm2 at 1 ft for influenza in water is detailed in the table below with various time points and corresponding doses and percentages of killed virus.

### Data and Calculations

Data includes far UVC device with 7.0 uW/cm2 at 1 meter killing 99% influenza on surface in 1 hour and 22% of enveloped viruses in a 3 k sq ft room in 30 minutes.

Far UVC device strength is 27.1 uW/cm2 at 1 foot per factory measurements.

Current measurement are set for the present far UVC device at 12.48 uW/cm for a 0.7 mJ/cm2 after 1 minute exposure directly under the light at 1 ft distance. These measurements and settings are based on related usages in the field (Song B M, et al., PLoS One. 2023 Nov. 28; 18(11):e0294427. Eadie E, et al., Sci Rep. 2022 Mar. 23; 12(1):4373).

A conservative D₉₀~19 mJ/cm2 is reported for influenza suspended in water at 222 nm. D₅₀ is approximately 0.301×D₉₀~5.7 mJ/cm2, and D99 is approximately 2×D₉₀~38 mJ/cm2. These are standard log-linear relationships for UV inactivation in liquids.

The percentage of killed virus (% killed) is calculated using the formula: 100×(1−10−Dose/D₉₀), where Dose comes from measured irradiance (uW/cm²)×time.

For the present dar UVC device at 1 ft in IBT measurements, 12.48 uW/cm2 equates to 0.7488 mJ/cm2 per minute. The kill curve was rebuilt using influenza-in-water UV data (D₉₀~19 mJ/cm2 at 222 nm) and measured irradiance at 1 ft to calculate dose per minute and the minute-by-minute kill.

### Samples and Assays

Samples include 0% (infectious stock), 50% inactivation (sample at 8-minute exposure), a mix of 0% and 100% (1:1 v/v=50-75%), and 100% inactivation.

A plaque assay is used to confirm inactivation and determine titers.

All samples are saved at −80° C. for back-titer and future ELISA to confirm viral protein presence.

TABLE 1
Kill Curve - Far UVC device set up for 12.48
μW/cm² at 1 ft distance (Influenza virus in Water)

| Time | Dose | % Virus |
|---|---|---|
| (min) | (mJ/cm²) | Killed |
| 0 | 0 | 0.00% |
| 1 | 0.75 | 8.80% |
| 2 | 1.5 | 16.90% |
| 3 | 2.25 | 24.10% |
| 4 | 3 | 30.60% |
| 5 | 3.74 | 36.50% |
| 6 | 4.49 | 41.80% |
| 7 | 5.24 | 46.60% |
| 8 | 5.99 | 50.90% |
| 9 | 6.74 | 54.90% |
| 10 | 7.49 | 58.40% |
| 11 | 8.24 | 61.70% |
| 12 | 8.99 | 64.70% |
| 13 | 9.73 | 67.40% |
| 14 | 10.48 | 69.90% |
| 15 | 11.23 | 72.20% |
| 16 | 11.98 | 74.30% |
| 17 | 12.73 | 76.20% |
| 18 | 13.48 | 77.90% |
| 19 | 14.23 | 79.60% |
| 20 | 14.98 | 81.00% |
| 21 | 15.73 | 82.40% |
| 22 | 16.48 | 83.70% |
| 23 | 17.22 | 84.80% |
| 24 | 17.97 | 85.90% |
| 25 | 18.72 | ≈90% (D₉₀) |
| 26 | 19.47 | 90.50% |
| 27 | 20.22 | 91.20% |
| 28 | 20.97 | 91.90% |
| 29 | 21.72 | 92.50% |
| 30 | 22.46 | 93.10% |
| 31 | 23.21 | 93.60% |
| 32 | 23.96 | 94.10% |
| 33 | 24.71 | 94.50% |
| 34 | 25.46 | 94.90% |
| 35 | 26.21 | 95.30% |
| 36 | 26.96 | 95.70% |
| 37 | 27.71 | 96.00% |
| 38 | 28.46 | 96.30% |
| 39 | 29.2 | 96.60% |
| 40 | 29.95 | 96.90% |
| 41 | 30.7 | 97.10% |
| 42 | 31.45 | 97.40% |
| 43 | 32.2 | 97.60% |
| 44 | 32.95 | 97.80% |
| 45 | 33.7 | 98.00% |
| 46 | 34.45 | 98.20% |
| 47 | 35.2 | 98.40% |
| 48 | 35.94 | 98.60% |
| 49 | 36.69 | 98.70% |
| 50 | 37.44 | ≈99% (D₉₉) |
| 51 | 38.19 | 99.10% |
| 52 | 38.94 | 99.20% |
| 53 | 39.69 | 99.30% |
| 54 | 40.44 | 99.40% |
| 55 | 41.18 | 99.45% |
| 56 | 41.93 | 99.50% |
| 57 | 42.68 | 99.55% |
| 58 | 43.43 | 99.60% |
| 59 | 44.18 | 99.65% |
| 60 | 44.93 | 99.70% |

### 2—Animal Challenge Study

The phase timeline includes the following:

Acclimation for 5-7 days, with BALB/c females housed 3/cage.

Pre-bleed on Day −1, with Retro-orbital bleed for baseline sera.

Challenge on Day 0, with Intranasal 35 μL dose per group as above.

At Day 3, a test bleed and interim sacrifice subset if needed.

At Day 6, a terminal bleed and lung harvest on Day 6.

The data record completion is set for Days 7-9.

Endpoints include body weight, clinical scores, survival, plaque assay titers, and cytokine profiles (Luminex).

### Sample Processing

Lungs are processed with the left lung snap-frozen for optional histopathology and the right lung homogenized for plaque assay and Luminex.

Serum is collected at Day −1, 3, and 6, and stored at −80° C.

Back-titer is performed on challenge dose vials to verify infection load.

For any post-study, all samples remain frozen for potential ELISA confirmation of viral protein integrity.

### Analytical Assays

A plaque assay is performed to determine infectious titer and to confirm inactivation. Analysis of cytokine and chemokine responses (Th1/Th2), and optionally ELISA is done to confirm the presence of inactivated viral proteins (HA/NP).

### Timeline

The timeline includes manual review and equipment setup (Week 1), dry run and plaque assay readout (Weeks 2-3), ordering mice (Week 4), acclimation and challenge in (Weeks 5-6), and Luminex/analysis in (Weeks 7-8).

### 3—Further Challenge and Immunogenicity

Additional studies are considered based on the below design:

### Change Animal Groups

| | | | n (female |
|---|---|---|---|
| | | | BALB/c, |
| Group | Description | Inactivation Condition | 6-8 weeks) |
| 1 | Control | 0% inactivation (live virus) | 3 |
| 2 | Partial | 50-75% inactivated (either | 3 + 2 = 5 |
| | | drawn at half-time or | |
| | | mixed 1:1 live:inactivated) | |
| 3 | Full | 100% inactivated | 3 + 2 = 5 |

### Timeline and Challenges

| Phase | Timeline (est.) | Details |
|---|---|---|
| Acclimation | 5-7 days | BALB/c females housed 3/cage. |
| Day −1 | Pre-bleed | Retro-orbital bleed for baseline |
| | | sera. |
| Day 0 | Challenge (Dose 1) | Intranasal 35 μL dose per group as |
| | | above. |
| Day 3 | Test bleed + interim sacrifice subset if | |
| | needed. | |
| Day 6 | Terminal bleed and lung harvest (or earlier if | |
| | moribund) for N = 3 animals per group | |
| | Endpoints: Body weight, clinical scores, | |
| | survival, plaque assay titers, cytokine | |
| | profiles (Luminex) | |
| Day 7-28* | Move N = 2 animals from Group 2 and 3 into | |
| | Immunogenicity protocol (Total N = 4 from | |
| | two groups) | |
| Day 14 | Immunize with another dose of 50% | |
| | inactivated and 99% inactivated dose for | |
| | Groups 2 and 3 (Dose 2) | |
| Day 27 | Test bleed for INFV ELISA to review | |
| | immunogenicity titers. Endpoints: Weekly | |
| | Body weight, clinical scores & INFV Titers | |
| Day 28 | Terminate OR Immunize again similar to | |
| | Day 14 (Dose 3) | |
| Day 41 | Test bleed for INFV ELISA to review | |
| | immunogenicity titers. Endpoints: Weekly | |
| | Body weight, clinical scores & INFV Titers | |

### Example 5: Comparative Assessment of Interleukin 8 Presentation by Lung Epithelial Cells with Far UVC Inactivated Coronavirus

Mammalian viruses that cause human respiratory disease induce a primary immune response in lung cells following inactivation with far UVC (222 nm (UV222)). Inactivation is defined here as removing infectious ability of a virus, without compromising the structural integrity of a UV exposed virion. While relatively low doses of UV222 are needed to inactivate many airborne human viruses (e.g. Influenza, Coronavirus, Measles etc.), the dominant inactivation mechanism associated with UV222 exposures remains tenuous.

Prior investigations indicate that dose-dependent UV222 damage of viral proteins required for successful lung infection are systematically damaged by UV222 exposure. Viral inactivation associated UV222 exposures has been theorized to include non-specific denaturing of “spike (surface) proteins” and capsid proteins of Coronaviruses. If this is the case, UV inactivated Coronavirus would not recognize or bind to surface receptors on lung cells and infection could not progress. However, lung cells may still recognize UV damaged proteins on the virion and although infection may not be successful, a non-specific immune response may ensue. If this is the case, UV222 may be a path for preparing live attenuated virus to stimulate a novel, non-threatening immune response via an aerosol route.

Vaccines of live attenuated viruses have been safely used for generations—such is the case with some Measles and Influenza vaccines in widespread use around the world. Live attenuated virus vaccines use genetically modified virions, have been used in conventional (liquid adjuvant) and aerosol forms. However, a UV modified analogue has not been used to date.

In response to this paucity, the purpose of this investigation is to determine if engineered UV222 exposure can simultaneously inactivate a common pathogenic respiratory virus, while at the same time preserve the virions' ability to induce a non-threatening immune response. In this context, a key biochemical initiator for respiratory immune function is the well-characterized protein, Interleukin 8. This soluble protein is secreted by epithelial lung cells during the early stages of infection. This Interleukin acts as a specific biochemical signal to mediate lung inflammatory response by attracting neutrophils and T-cells to infection sites.

Interleukins in standardized lung cell culture is assessed to determine whether UV222 inactivated mammalian Coronavirus can retain its ability it initiate the immune cascade without experiencing infection. Widely accepted UV disinfection practices, cell culture methods and biochemical assays are used to compare interleukin release patterns of virus infected lung cells with otherwise identical virus inactivate with UV.

### Example 6: Viral Inactivation Using Beacon Light

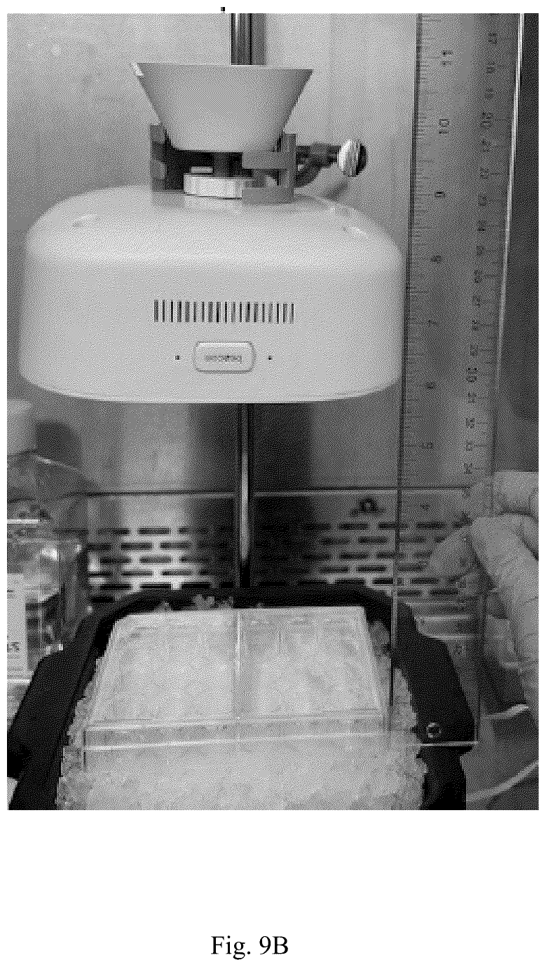

To assess viral inactivation using a continuous Far-UVC 222 nm disinfection system as described herein (e.g., Beacon light), a standard plaque assay with mouse-adapted Influenza A/Puerto Rico/8/1934 (INFV A/PR/8/34 or H1N1) was performed. Viruses were inactivated using the Beacon light under four conditions: 0% inactivated (no exposure to Beacon light; “Condition 1”), 50% inactivated by time (8-minute exposure to Beacon light; “Condition 2), 100% inactivated (50-minute exposure to Beacon light; “Condition 4”), or 50% inactivated by volume (1:1 mixture of 0% inactivated sample and 100% inactivated sample; “Condition 3”). To inactivate the virus, 2×10⁵ PFU/mL of INFV A/PR/8/34 was prepared in a working dilution of 1:10. For Condition 1, 300 μL of diluted INFV A/PR/8/34 was untreated and kept on ice. For Conditions 2-4, 700 μL of diluted INFV A/PR/8/34 was transferred to a 24-well plate on ice and exposed to the Beacon light suspended 12 inches above plate (FIG. 9A). The plate was mixed at 4 min, 8 min, 15 min, 30 min, and 45 min.

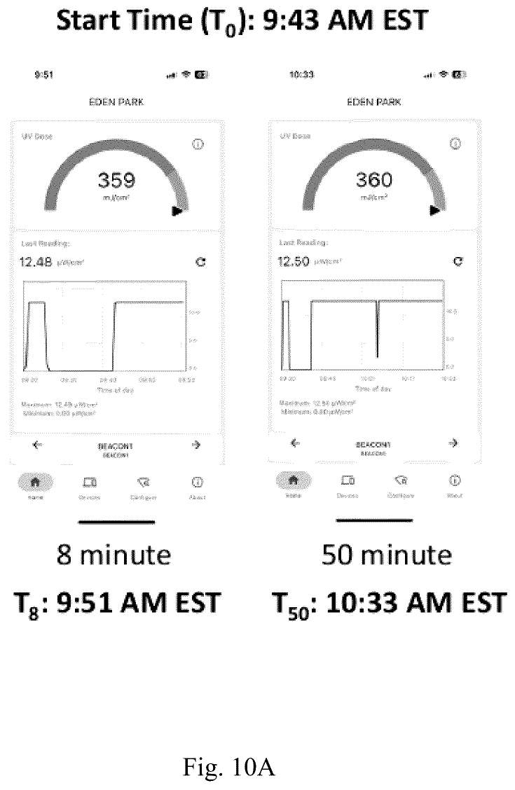

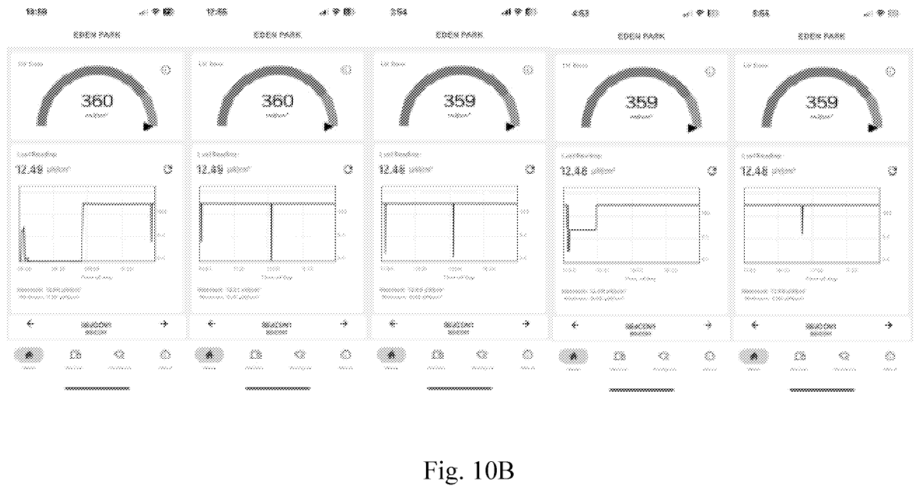

To evaluate the viral titer after exposure to the Beacon light, samples were serially diluted and added to Vero cells seeded the day prior in 24-well plates. After a 1-hour incubation, a methylcellulose overlay was added to the cell monolayer to prevent virus spread, which causes plaques to form. Each virus has a predetermined range of time for plaque formation. Plaques were immuno-stained and quantified with a plaque counter. The viral titer was then calculated in Plaque Forming Units (PFU)/mL. The results are shown in Table 2 and FIGS. 10A and 10B.

TABLE 2
Viral Titer after Exposure to Beacon Light

| | | | Inactivation | Inactivation |
|---|---|---|---|---|
| | | Total # of plaques | relative to | relative to |
| | Titer | (across 4 dilutions, | Condition #1 | Condition #1 |
| Condition | (PFU/mL) | n = 3) | (%) | (log ratio) |
| 1 | 2.07 × 10⁴ | 690 | — | — |
| 2 | 1.66 × 10⁴ | 552 | 20% | 0.10 |
| 3 | 1.47 × 10⁴ | 489 | 29% | 0.15 |
| 4 | 1.12 × 10⁴ | 373 | 46% | 0.27 |

The results in Table 2 show that a 50-minute exposure of INFV A/PR/8/34 to the Beacon light (Condition 4) led to a 46% decrease in titer compared to the untreated control (Condition 1).

### Example 7: Immune Response in Mice Exposed to Beacon-Inactivated Influenza Virus

To evaluate if Beacon light-inactivated influenza virus can generate humoral immune responses, a pilot study in mice was conducted. Briefly, to inactivate the virus, a formulation containing 1:10 stock of mouse-adapted INFV A/PR/8/34 (2.5×10⁵ PFU) was prepared in a 1 mL volume and transferred to a 24-well plate on ice positioned 6 inches under the Beacon light for a total of 8 hours, mixed by pipetting each hour (FIG. 9B). Following the 8-hour inactivation period, a final immunization formulation was prepared for administration. Mice (n=3) were intranasally administered 35 μL of the final immunization formulation. Throughout the study, all mice were healthy, displaying smooth coats and bright eyes and appearing alert with active behavior including scurrying and burrowing.

On Days 14 and 28, serum samples were taken and assessed for the presence of anti-INFV A/PR/8/34 antibodies by ELISA. Briefly, plates were coated with INFV A/PR/8/34 in DPBS for one hour. Following the hour incubation, plates were washed with ELISA wash buffer (DPBS+0.05% TWEEN20) and blocked for an hour with StartingBlock (PBS) blocking buffer. Following the blocking step, plates were washed and the serum samples were serially diluted semi-log and incubated on the plate for one hour. Following the one-hour incubation, plates were washed and goat anti-mouse IgG (H+L) HRP secondary antibody was added to the plate. Following the secondary antibody incubation, plates were washed and TMB substrate was added to the plate. Optical density was measured with a microplate reader. Data analysis and visualization was performed in GraphPad Prism. The results for raw absorbance at OD650 are shown in Table 3, and the signal-to-noise (S/N) ratio calculated using the background well is shown in Table 4. An positive control antibody (anti-INFV antibody) was observed within the expected range with an average S/N of 17.02 at Day 14 and 20.68 at Day 28.

TABLE 3
Absorbance Values OD650

| | | Animal ID 371 | | Animal ID 793 | | Animal ID 794 | |
|---|---|---|---|---|---|---|---|
| Day | Dilution | OD650 | StDev | OD650 | StDev | OD650 | StDev |
| 14 | 15 | 2.29 | 0.01 | 2.46 | 0.01 | 2.48 | 0.06 |
| | 47 | 2.32 | 0.14 | 2.48 | 0.00 | 2.53 | 0.15 |
| | 150 | 2.16 | 0.16 | 2.08 | 0.05 | 2.30 | 0.26 |
| | 473 | 1.72 | 0.06 | 1.63 | 0.01 | 1.74 | 0.08 |
| | 1496 | 1.17 | 0.03 | 1.04 | 0.11 | 1.22 | 0.04 |
| | 4726 | 0.63 | 0.05 | 0.47 | 0.02 | 0.61 | 0.00 |
| | 14935 | 0.28 | 0.01 | 0.20 | 0.00 | 0.26 | 0.01 |
| | 47196 | 0.08 | 0.03 | 0.05 | 0.01 | 0.05 | 0.00 |
| 28 | 15 | 2.38 | 0.01 | 2.48 | 0.08 | 2.44 | 0.02 |
| | 47 | 2.33 | 0.04 | 2.37 | 0.00 | 2.44 | 0.00 |
| | 150 | 2.32 | 0.03 | 2.28 | 0.00 | 2.38 | 0.02 |
| | 473 | 2.22 | 0.02 | 2.09 | 0.01 | 2.29 | 0.03 |
| | 1496 | 2.02 | 0.02 | 1.78 | 0.04 | 2.13 | 0.01 |
| | 4726 | 1.64 | 0.01 | 1.21 | 0.00 | 1.77 | 0.02 |
| | 14935 | 1.05 | 0.03 | 0.61 | 0.00 | 1.17 | 0.02 |
| | 47196 | 0.53 | 0.01 | 0.29 | 0.01 | 0.59 | 0.01 |

TABLE 4
Signal to Noise Ratio Calculated using Background well (S/N)

| Day | Dilution | Animal ID 371 | Animal ID 793 | Animal ID 794 |
|---|---|---|---|---|
| 14 | 15 | 39.72 | 42.56 | 42.91 |
| | 47 | 40.13 | 43.00 | 43.83 |
| | 150 | 37.41 | 35.97 | 39.77 |
| | 473 | 29.81 | 28.21 | 30.08 |
| | 1496 | 20.26 | 17.98 | 21.06 |
| | 4726 | 10.95 | 8.17 | 10.50 |
| | 14935 | 4.92 | 3.52 | 4.53 |
| | 47196 | 1.37 | 0.83 | 0.79 |
| 28 | 15 | 56.81 | 59.07 | 58.20 |
| | 47 | 55.59 | 56.63 | 58.31 |
| | 150 | 55.25 | 54.31 | 56.75 |
| | 473 | 52.89 | 49.95 | 54.51 |
| | 1496 | 48.11 | 42.45 | 50.78 |
| | 4726 | 39.12 | 28.85 | 42.32 |
| | 14935 | 25.06 | 14.64 | 27.96 |
| | 47196 | 12.62 | 6.94 | 14.02 |

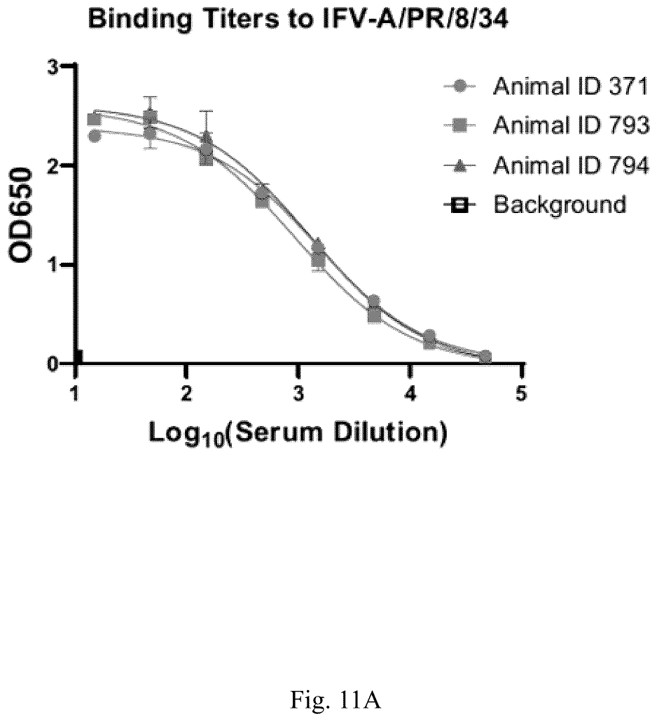

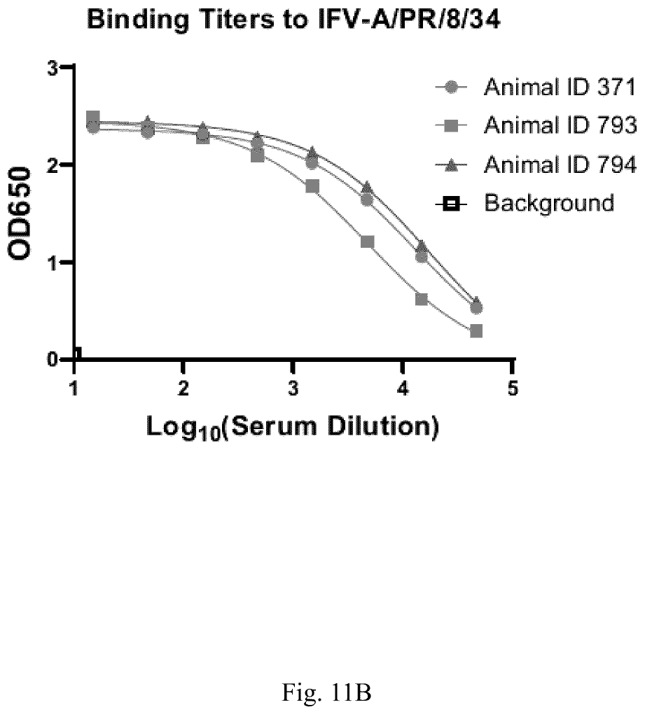

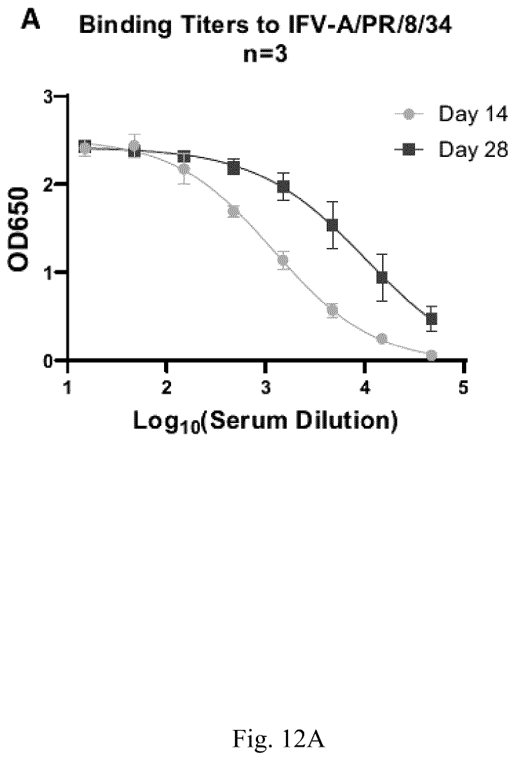

The data shows that all three animals developed an immunogenic response to mouse-adapted INFV A/PR/8/34 virus inactivated by the Beacon light, with robust OD650 values and S/N ratios compared to background. A sigmoidal binding curve for all three animals are shown in FIG. 11A (Day 14) and FIG. 11B (Day 28) compared to background.

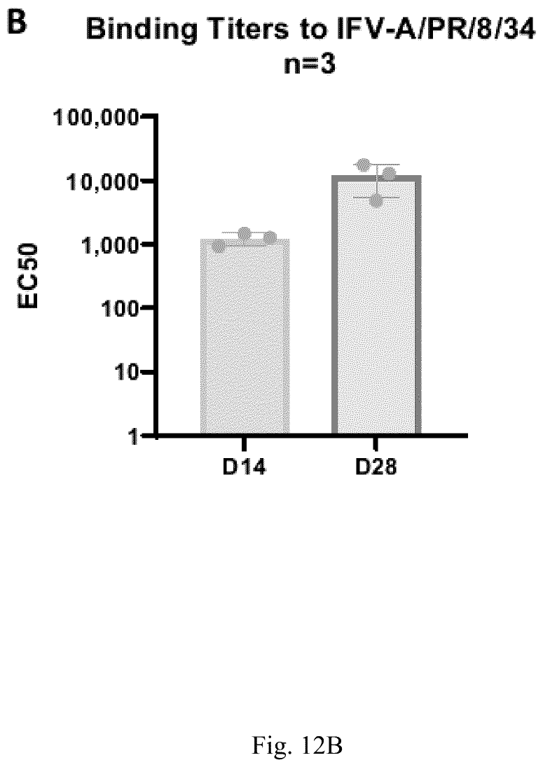

A comparison of the average OD650 for three mice between Day 14 and Day 28 is shown in FIG. 12A. The binding profiles were used to derive mean EC₅₀ values (half-maximal effective concentration) using 4-parameter logistic regression in GraphPad Prism. As shown in FIG. 12B, the immunogenic titers increased ~10-fold from Day 14 (EC₅₀ Mean=1225.9) to Day 28 (EC₅₀ Mean=11,787.3). Overall, the results presented herein demonstrate that Beacon light-inactivated influenza virus can stimulate robust humoral immune responses in mice. Mice intranasally exposed to Beacon light-inactivated INFV A/PR/8/34 exhibited detectable antigen-specific antibodies at multiple post-exposure time points, including Day 14 and Day 28. Antibody levels at Day 28 were greater than those observed at Day 14, indicating persistence and continued development of the immune response over time. The presence of antigen-specific antibodies at these later time points is consistent with an adaptive immune response and supports the generation of immunological memory.

### Example 8: Immune Response in Mice Exposed to Beacon-Inactivated RSV

To evaluate if Beacon light-inactivated respiratory syncytial virus (RSV) can generate humoral immune responses, a study in mice was conducted. Briefly, to inactivate the virus, a formulation containing 1:10 stock of RSV (2.37×10⁵ PFU) was prepared in a 1 mL volume and transferred to a 24-well plate on ice positioned 6 inches under the Beacon light for a total of 8 hours, mixed by pipetting each hour. Following the 8-hour inactivation period, a final immunization formulation was prepared for administration. Mice (n=3) were intranasally administered 35 μL of the final immunization formulation. Throughout the study, all mice were healthy, displaying smooth coats and bright eyes and appearing alert with active behavior including scurrying and burrowing.

On Days −1, 13, and 28, serum samples were taken and assessed for the presence of anti-RSV antibodies by ELISA. Briefly, plates were coated with RSV in DPBS for one hour. Following the hour incubation, plates were washed with ELISA wash buffer (DPBS+0.05% TWEEN20) and blocked for an hour with StartingBlock (PBS) blocking buffer. Following the blocking step, plates were washed and the serum samples were serially diluted semi-log and incubated on the plate for one hour. Following the one-hour incubation, plates were washed and goat anti-mouse IgG (H+L) HRP secondary antibody was added to the plate. Following the secondary antibody incubation, plates were washed and TMB substrate was added to the plate. Optical density was measured with a microplate reader. Data analysis and visualization was performed in GraphPad Prism. The results for average absorbance at OD650 across two technical replicates are shown in Table 5. An positive control antibody (anti-RSV Antibody, fusion protein, all type A, B strains, 25 μg/mL) was observed within the expected range with an average OD650 of 0.30035 at Day 13 and 0.28135 at Day 28. Overall, the results presented herein demonstrate that Beacon light-inactivated RSV can stimulate robust humoral immune responses in mice. Mice intranasally exposed to Beacon light-inactivated RSV exhibited detectable antigen-specific antibodies at multiple post-exposure time points, including Day 14 and Day 28. Antibody levels at Day 28 were greater than those observed at Day 14, indicating persistence and continued development of the immune response over time. The presence of antigen-specific antibodies at these later time points is consistent with an adaptive immune response and supports the generation of immunological memory.

TABLE 5
Average Absorbance Values OD650

| Day | Dilution | Pooled samples (n = 3) |
|---|---|---|
| −1 | 15 | 0.08905 |
| | 47 | 0.06265 |
| | 150 | 0.05285 |
| | 473 | 0.0461 |
| | 1496 | 0.0453 |
| | 4726 | 0.04595 |
| | 14935 | 0.0431 |
| | 47196 | 0.04465 |

| Day | Dilution | Animal ID 198 | Animal ID 199 | Animal ID 200 |
|---|---|---|---|---|
| 14 | 15 | 2.9193 | 2.8651 | 2.90505 |
| | 47 | 2.8169 | 2.85615 | 2.8295 |
| | 150 | 2.5433 | 2.7106 | 2.67045 |
| | 473 | 2.03665 | 2.428 | 2.248 |
| | 1496 | 1.2316 | 1.78505 | 1.5261 |
| | 4726 | 0.56795 | 1.00985 | 0.7503 |
| | 14935 | 0.229 | 0.42935 | 0.29135 |
| | 47196 | 0.11085 | 0.19635 | 0.13385 |
| 28 | 15 | 3.1145 | 3.09975 | 3.07995 |
| | 47 | 2.91075 | 3.0082 | 2.8952 |
| | 150 | 2.2986 | 2.6961 | 2.33765 |
| | 473 | 1.4406 | 2.0592 | 1.5168 |
| | 1496 | 0.68275 | 1.211 | 0.7878 |
| | 4726 | 0.30025 | 0.5671 | 0.35755 |
| | 14935 | 0.1369 | 0.25065 | 0.16185 |
| | 47196 | 0.104 | 0.13615 | 0.08435 |

### Example 9: Immune Response in Mice Exposed to Beacon-Inactivated Influenza: Expanded

To evaluate if Beacon-inactivated influenza virus can generate humoral immune responses, an expanded study in mice was conducted. Briefly, to inactivate the virus, a formulation containing 1:10 stock of mouse-adapted INFV A/PR/8/34 (2.5×10⁵ PFU) was prepared in a 1 mL volume and transferred to a 24-well plate on ice positioned 6 inches under the Beacon light for a total of 8 hours, mixed by pipetting each hour. Following the 8-hour inactivation period, a final immunization formulation was prepared for administration. The study design for the expanded influenza study is shown in Table 6.

TABLE 6
Study Design for Expanded Influenza Study

| | | | Volume/ |
|---|---|---|---|
| Group | Subgroup | Treatment | Route |
| 1 | 1A (n = 1) | 1x DPBS | 35 uL via |
| | 1B (n = 4) | Beacon light-inactivated mouse-adapted | intranasal |
| | | INFV A/PR/8/34 (400 PFU/mouse) | instillation |
| 2 | 2A (n = 1) | 1x DPBS | |
| | 2B (n = 4) | Beacon light-inactivated mouse-adapted | |
| | | INFV A/PR/8/34 (400 PFU/mouse) | |
| 3 | 3A (n = 1) | 1x DPBS | |
| | 3B (n = 4) | Beacon light-inactivated mouse-adapted | |
| | | INFV A/PR/8/34 (400 PFU/mouse) | |

For each group in Table 6, the mouse designated in the A subgroup (receiving 1×DPBS) will be co-housed with the four mice in the corresponding B subgroup in the same cage after intranasal instillation. Throughout the study, all mice were healthy, displaying smooth coats and bright eyes and appearing alert with active behavior including scurrying and burrowing.

On Days −1, Day 13, and Day 28, serum samples were taken and assessed for the presence of anti-INFV A/PR/8/34 antibodies by ELISA. Briefly, plates were coated with INFV A/PR/8/34 in DPBS for one hour. Following the hour incubation, plates were washed with ELISA wash buffer (DPBS+0.05% TWEEN20) and blocked for an hour with StartingBlock (PBS) blocking buffer. Following the blocking step, plates were washed and the serum samples were serially diluted semi-log and incubated on the plate for one hour. Following the one-hour incubation, plates were washed and goat anti-mouse IgG (H+L) HRP secondary antibody was added to the plate. Following the secondary antibody incubation, plates were washed and TMB substrate was added to the plate. Optical density was measured with a microplate reader. Data analysis and visualization was performed in GraphPad Prism. The results for average absorbance at OD650 across two technical replicates are shown in Table 7. An positive control antibody (anti-INFV monoclonal antibody) was observed within the expected range with an average OD650 of 1.19775 at Day 13 and 1.2941 on Day 28. Overall, the results presented herein demonstrate that Beacon light-inactivated INFV A/PR/8/34 can stimulate robust humoral immune responses in mice.

TABLE 7
Average Absorbance Values OD650
at Day −1, Day 13, and Day 28

| Day | Dilution | Pooled samples (n = 15) |
|---|---|---|
| −1 | 15 | 0.0917 |
| | 47 | 0.0588 |
| | 150 | 0.04845 |
| | 473 | 0.0452 |
| | 1496 | 0.0445 |
| | 4726 | 0.04345 |
| | 14935 | 0.0594 |
| | 47196 | 0.1183 |

| Group | Dilution | 1A | 1B-1 | 1B-2 | 1B-3 | 1B-4 |
|---|---|---|---|---|---|---|
| 1 | 15 | 2.9193 | 2.241 | 2.12575 | 2.0842 | 2.0663 |
| Day 13 | 47 | 2.8169 | 2.0544 | 1.9409 | 1.9724 | 1.9739 |
| | 150 | 2.5433 | 1.8386 | 1.6318 | 1.78165 | 1.74405 |
| | 473 | 2.03665 | 1.50215 | 1.18905 | 1.4486 | 1.4452 |
| | 1496 | 1.2316 | 1.0127 | 0.6553 | 0.92835 | 0.9316 |
| | 4726 | 0.56795 | 0.5661 | 0.32545 | 0.51455 | 0.4979 |
| | 14935 | 0.229 | 0.26875 | 0.1563 | 0.2431 | 0.23835 |
| | 47196 | 0.11085 | 0.15435 | 0.12095 | 0.1382 | 0.1444 |
| Group | Dilution | 2A | 2B-1 | 2B-2 | 2B-3 | 2B-4 |
| 2 | 15 | 0.10395 | 2.04575 | 1.9283 | 2.25415 | 2.0367 |
| Day 13 | 47 | 0.0715 | 1.8898 | 1.75025 | 2.15155 | 1.88945 |
| | 150 | 0.05315 | 1.62635 | 1.45005 | 1.93345 | 1.66705 |
| | 473 | 0.0479 | 1.2445 | 1.062 | 1.6582 | 1.369 |
| | 1496 | 0.0444 | 0.7229 | 0.58755 | 1.12935 | 0.9816 |
| | 4726 | 0.04525 | 0.35905 | 0.29025 | 0.6201 | 0.50195 |
| | 14935 | 0.04605 | 0.17835 | 0.14005 | 0.29475 | 0.2302 |
| | 47196 | 0.0435 | 0.0942 | 0.08665 | 0.20125 | 0.1498 |
| Group | Dilution | 3A | 3B-1 | 3B-2 | 3B-3 | 3B-4 |
| 3 | 15 | 0.1004 | 2.019 | 2.1432 | 2.07015 | 1.9925 |
| Day 13 | 47 | 0.0689 | 1.7876 | 1.9563 | 1.8818 | 1.8638 |
| | 150 | 0.05295 | 1.53855 | 1.7177 | 1.54095 | 1.61325 |
| | 473 | 0.0465 | 1.1997 | 1.3868 | 1.14605 | 1.2916 |
| | 1496 | 0.04275 | 0.77795 | 0.902 | 0.6503 | 0.84455 |
| | 4726 | 0.0428 | 0.4272 | 0.50155 | 0.33495 | 0.4774 |
| | 14935 | 0.04375 | 0.21185 | 0.23795 | 0.1587 | 0.2403 |
| | 47196 | 0.0442 | 0.14265 | 0.1487 | 0.1475 | 0.12615 |
| Group | Dilution | 1A | 1B-1 | 1B-2 | 1B-3 | 1B-4 |
| 1 | 15 | 0.09395 | 2.062 | 2.09205 | 2.15215 | 2.3023 |
| Day 28 | 47 | 0.12055 | 2.0161 | 2.06805 | 2.09495 | 2.1948 |
| | 150 | 0.0624 | 1.9742 | 1.9307 | 1.9545 | 2.09525 |
| | 473 | 0.1914 | 1.80385 | 1.7666 | 1.7954 | 1.9243 |
| | 1496 | 0.0536 | 1.51885 | 1.37545 | 1.5125 | 1.5786 |
| | 4726 | 0.08845 | 1.0511 | 0.92135 | 1.0993 | 1.0945 |
| | 14935 | 0.04855 | 0.6329 | 0.49105 | 0.644 | 0.59835 |
| | 47196 | 0.04635 | 0.36765 | 0.25795 | 0.35295 | 0.31735 |
| Group | Dilution | 2A | 2B-1 | 2B-2 | 2B-3 | 2B-4 |
| 2 | 15 | 0.28985 | 2.02735 | 1.97515 | 2.1209 | 2.09315 |
| Day 28 | 47 | 0.12885 | 1.9287 | 1.9064 | 1.9887 | 2.0121 |
| | 150 | 0.0808 | 1.82405 | 1.77525 | 1.86105 | 1.9185 |
| | 473 | 0.05485 | 1.67435 | 1.57415 | 1.69495 | 1.76315 |
| | 1496 | 0.05255 | 1.4402 | 1.20435 | 1.4152 | 1.5051 |
| | 4726 | 0.04425 | 1.01735 | 0.7507 | 1.00255 | 1.09605 |
| | 14935 | 0.05 | 0.6089 | 0.40675 | 0.61895 | 0.67105 |
| | 47196 | 0.0444 | 0.3295 | 0.2218 | 0.36995 | 0.3909 |
| Group | Dilution | 3A | 3B-1 | 3B-2 | 3B-3 | 3B-4 |
| 3 | 15 | 0.10425 | 2.00465 | 2.09765 | 2.115 | 2.15995 |
| Day 28 | 47 | 0.0881 | 1.88925 | 2.0336 | 1.973 | 2.0861 |
| | 150 | 0.06105 | 1.77525 | 1.8829 | 1.8314 | 1.96195 |
| | 473 | 0.0483 | 1.542 | 1.7141 | 1.6037 | 1.8224 |
| | 1496 | 0.04925 | 1.2117 | 1.37525 | 1.18875 | 1.5518 |
| | 4726 | 0.04525 | 0.7762 | 1.02855 | 0.7083 | 1.15635 |
| | 14935 | 0.04585 | 0.4076 | 0.6197 | 0.36015 | 0.7113 |
| | 47196 | 0.04715 | 0.20745 | 0.33825 | 0.2124 | 0.37975 |

Overall, the results presented herein further demonstrate that Beacon light-inactivated influenza can stimulate robust humoral immune responses in mice. Taken together with Examples 7 and 8, the data demonstrate that exposure to Beacon-inactivated substrates comprising microbes, microbial particles, or other immunogenic materials induces an immune response in a subject. Specifically, a positive ELISA signal indicates the presence of antigen-specific antibodies directed against the administered antigen (e.g., influenza or RSV), with signal magnitude reflecting a measurable, statistically significant increase relative to baseline, consistent with antigen recognition and antibody generation.

Furthermore, the observed antibody response is attributable to the administered Beacon-inactivated antigen and not to environmental or experimental confounders. Negative control groups, including (i) Day 0 baseline samples and (ii) subjects administered Beacon-treated saline, exhibited minimal to no detectable antigen-specific antibody signal. Notably, control subjects were co-housed with antigen-exposed subjects, thereby controlling for potential environmental exposure (e.g., airborne particles, cage conditions, diet, or handling). The absence of antibody generation in these controls demonstrates that immune activation requires direct exposure to the Beacon light-inactivated antigen and is not induced by incidental environmental factors.

Without being bound by theory, these data demonstrate that Beacon light-mediated inactivation preserves sufficient antigenic structure to enable immune system recognition while eliminating infectivity, thereby enabling safe induction of an acquired immune response following exposure via non-invasive routes (e.g., intranasal administration). Collectively, the results support the claimed methods of triggering, enhancing, or providing anti-pathogen immunity through exposure to ultraviolet-inactivated compositions delivered via air and/or surface-mediated pathways.

### Example 10: Comparison of Stimulated Humoral Immune Responses Across Different Pathogens

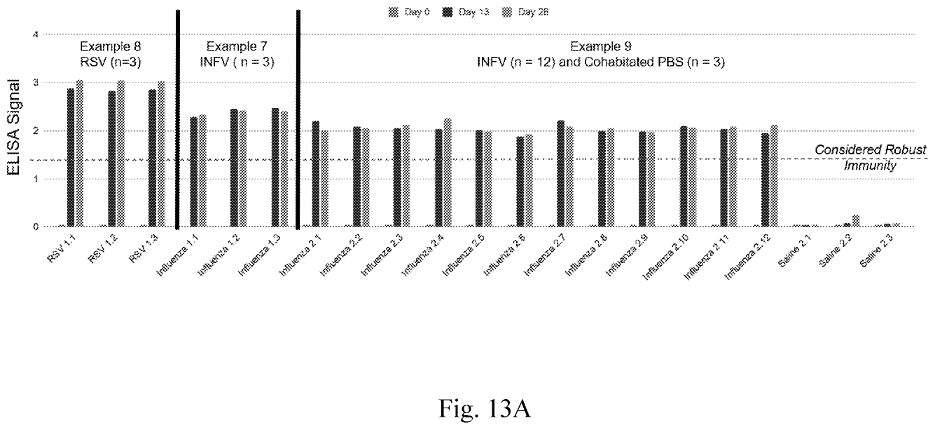

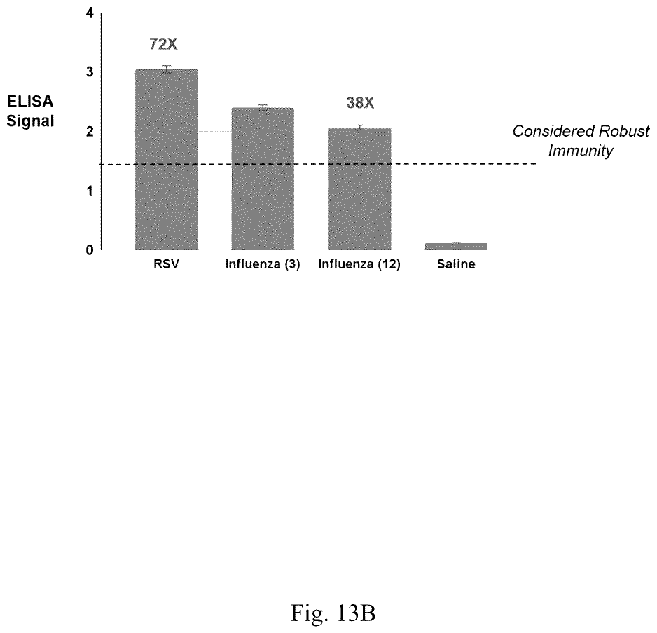

As described in Examples 7-9, mice received intranasal exposure to Beacon light-inactivated virus and the stimulated antibody responses were measured by Elisa. A comparison of each mouse over time is shown in FIG. 13A and Table 8 below. Not only did all animals remain clinically healthy throughout the study period, but mice developed strong polyclonal antibody responses to Beacon light-inactivated virus. The results suggest that Beacon-inactivated viruses retain their antigenic structure, allowing the immune system to recognize them and produce antibodies. The results demonstrate that Beacon light-inactivated virus generates robust antibody responses in mice without inducing illness. The response is consistent across viruses, replicates, and controls (FIG. 13B). Together, the Examples presented herein demonstrate proof-of-concept for environmental immune priming. The Beacon light converts infectious virus into a safe immune stimulus, which can enable “environmental immunity.”

TABLE 8
Relative ELISA Signal Across Mice Treated with Inactivated Virus

| | | | | Fold-Change |
|---|---|---|---|---|
| Sample | Day 0 | Day 13 | Day 28 | Day 0 vs 28 |
| RSV 1.1 | 0.04 | 2.87 | 3.06 | 72.44 |
| RSV 1.2 | 0.04 | 2.82 | 3.05 | 72.10 |
| RSV 1.3 | 0.04 | 2.86 | 3.03 | 71.63 |
| Influenza 1.1 | — | 2.29 | 2.34 | — |
| Influenza 1.2 | — | 2.46 | 2.43 | — |
| Influenza 1.3 | — | 2.48 | 2.40 | — |
| Influenza 2.1 | 0.05 | 2.20 | 2.02 | 37.96 |
| Influenza 2.2 | 0.05 | 2.09 | 2.05 | 38.53 |
| Influenza 2.3 | 0.05 | 2.05 | 2.11 | 39.66 |
| Influenza 2.4 | 0.05 | 2.03 | 2.26 | 42.48 |
| Influenza 2.5 | 0.05 | 2.01 | 1.99 | 37.33 |
| Influenza 2.6 | 0.05 | 1.89 | 1.94 | 36.35 |
| Influenza 2.7 | 0.05 | 2.22 | 2.08 | 39.09 |
| Influenza 2.8 | 0.05 | 2.00 | 2.05 | 38.56 |
| Influenza 2.9 | 0.05 | 1.98 | 1.97 | 36.93 |
| Influenza 2.10 | 0.05 | 2.10 | 2.06 | 38.68 |
| Influenza 2.11 | 0.05 | 2.03 | 2.08 | 39.01 |
| Influenza 2.12 | 0.05 | 1.95 | 2.12 | 39.85 |
| Saline 2.1 | 0.05 | 0.05 | 0.05 | 1.00 |
| Saline 2.2 | 0.05 | 0.07 | 0.25 | 4.69 |
| Saline 2.3 | 0.05 | 0.06 | 0.07 | 1.24 |

### Example 11: Aerosolized Delivery of Beacon-Inactivated Pathogens in a Controlled Environment

A study will be performed to evaluate whether aerosolized Beacon light-inactivated pathogens elicit humoral immune responses comparable to intranasal instillation. A subject (e.g., a mouse) will be evaluated for baseline immune status by obtaining a pre-exposure (Day 0) blood sample. The subject will subsequently be placed within a controlled environment chamber comprising regulated airflow. A UVC-emitting device (e.g., a Beacon light system) is positioned to irradiate at least a portion of the air volume within the chamber.

A pathogen (e.g., a virus) will be introduced into the chamber airspace as an aerosol, simulating natural transmission pathways (e.g., respiratory droplets from coughing or sneezing). The quantity of aerosolized particles may range from a single infectious unit (e.g., 1 PFU/CFU), to higher concentrations limited only by the physical constraints of the environment. Upon exposure to UVC radiation, the pathogen is expected to be inactivated via one or more mechanisms, including but not limited to nucleic acid damage, protein modification, membrane disruption, or structural alteration, thereby reducing or eliminating infectivity while retaining immunogenic components.

The resulting inactivated particles (or antigenic fragments thereof) are available for uptake by the subject via one or more exposure routes, including inhalation (nasal/oral), ocular exposure, or mucosal contact. The extent of exposure may vary from a single particle or fragment thereof to the entirety of available inactivated particles within the exposure environment.

Post-exposure immune responses will be assessed at one or more time points (e.g., Days 14 and 28) by collecting blood samples. Antigen-specific antibody production will be evaluated using immunoassays, including but not limited to Western blot and/or ELISA. Detection reagents which target whole-pathogen antigens will be used, thereby enabling identification of antibodies directed against any antigenic component of the pathogen. The results will be compared against appropriate controls, including (i) baseline (Day 0) samples, (ii) negative controls (e.g., saline-exposed subjects), and (iii) assay-provided positive controls, to confirm the specificity and validity of the immune response.

Additional endpoints will include assessment of tissue-level immune responses. For example, respiratory tissues (e.g., lungs) will be collected at defined time points (e.g., Days 13 and 28) to evaluate inflammation, cytokine expression, and other physiological indicators of immune activation. Functional characterization of the antibody response may be performed using assays such as hemagglutination inhibition (HAI) and microneutralization assays, which assess the ability of subject-derived antibodies to neutralize live pathogens in vitro and prevent infection of susceptible cells.

Without being bound by any particular theory, the data presented in the above Examples supports that UVC-inactivated pathogens will retain sufficient antigenic integrity to induce both binding and functional (e.g., neutralizing) antibody responses, consistent with activation of adaptive immunity.

Additional studies will be performed which assess a wide range of experimental and real-world conditions. For example, exposure may occur in enclosed or open environments (e.g., chambers, indoor spaces, outdoor settings), under varying ventilation conditions, and with differing modes of delivery (e.g., passive aerosol exposure, directed airflow, masks, vaporizers, or sprays). The systems described herein operate in the presence or absence of active pathogen shedding sources. Parameters which will be varied include exposure duration (ranging from acute to chronic), pathogen concentration (from a single PFU/CFU or fragment thereof to high environmental loads), number and configuration of UVC sources, and angles or geometry of irradiation.

In addition, pathogens will be introduced as pre-inactivated material or may be inactivated in situ within the environment in real time. The methods described herein are applicable across multiple species possessing an immune system and are not limited to any particular pathogen type, route of exposure, or environmental condition.

### Example 12: Calculation of a Pure Count of Inactivated Particles Through Dual-Channel Analysis

To evaluate the pure count of inactivated particles in an aerosolization chamber, a dual-channel analysis test will be conducted, simultaneously counting total particles and viable particles. Briefly, using an Aerodynamic Particle Sizer (APS) or an Optical Particle Counter (OPC), each physical particle in the air within a specific size range (e.g., 0.5 to 5.0 microns, which encompasses live viruses, Far-UVC inactivated viruses, and inert dust) will be measured. At the same time, using a Bioaerosol Sampler (e.g., Andersen Impactor or a BioSampler), the particles will be collected into a liquid medium which is subsequently cultured and tested using a Plaque Assay. The results of this assay will demonstrate how many particles are alive and/or infectious. To calculate the pure inactivated particle count, the viable particles (e.g., as measured by culture/plaque assay) will be subtracted from the total particles (e.g., as measured by APS or OPC) to determine the inactivated particle count.

Measurements of pure inactivated particles as described herein will be used to determine the amount of inactivated pathogen needed to elicit an immune response in a subject. For example, the tipping point of when immunity occurs may be based on one or more of the following factors in a room, test chamber, or facility, each of which ranges from a single unit measurement a maximum value potentially achievable under given conditions: volume of air; volume/amount of a selected virus/particle; identity of pathogen; amount of pathogen present; amount of pathogen inactivated or dosed; amount of inactivated pathogen is in the room (or alternatively, the breathing zone) that is exposed to a subject; volume of inactivated pathogen that may promote an immune response, e.g., the “tipping point,” which differs depending on the pathogen itself, including its structure and attributes; and the number of times a subject is exposed, at various concentrations, over a given timeframe (e.g., single, daily, etc.).

### REFERENCES

1. Kowalski, W. et al. (2009). Ultraviolet Germicidal Irradiation Handbook—UV inactivation mechanisms and broad-spectrum efficacy (Browne, K. Appl. Microbiol. 2021, 1, 537-556) (Browne, K. Appl. Microbiol. 2021, 1, 537-556).
2. MDPI—Browne, K. (2021). Mechanism of UV Microbial Inactivation (Review): discusses DNA damage (thymine/uracil dimers, 6-4 photoproducts) and protein effects (Browne, K. Appl. Microbiol. 2021, 1, 537-556) (Browne, K. Appl. Microbiol. 2021, 1, 537-556).
3. Terra Universal (2023). UVC vs Far-UVC: Safety, Applications, Effectiveness—industry analysis of 254 nm vs 222 nm: germicidal peak at 265 nm, far-UVC's safety and slightly lower efficiency (UVC vs Far-UVC: Analysis of Safety, Applications, and Microbial Effectiveness) (UVC vs Far-UVC: Analysis of Safety, Applications, and Microbial Effectiveness).
4. Woods, C. et al. (2020). Far-UVC Light Safely Kills Airborne Coronaviruses (Nature Scientific Reports): demonstrated 222 nm effectively inactivates aerosolized pathogens safely (Everything You Need to Know About Far-UVC Light) (Everything You Need to Know About Far-UVC Light).
5. Pusan Nat'l Univ. (2023). Filter-Free Far-UVC Device Study: notes 254 nm's harm to skin/eyes and development of safe 207 nm lamp (Filter-Free, Harmless, and Single-Wavelength Far UV-C Germicidal Light for Reducing Airborne Pathogenic Viral Infection—PMC).
6. USDA Study (2024). 254 nm vs 222 nm on Fungal Spores: both inactivate molds; far-UVC worked under ambient light (no photoreactivation needed) (UVC (254 nm) and Far UVC (222 nm) Irradiation Affects In Vitro Growth of Colletotrichum sp. Isolates and Their Infection of Detached Strawberry Leaves|PhytoFrontiers™)
7. Regency Supply (2021). UV-C and Far-UVC Overview: outlines practical pros/cons of conventional UVC (fast, proven, but harmful) and far-UVC development and safety testing (What's the difference between UV-C and Far-UVC?).
8. Lockwood, D. (2015). Bacteria May Remain Dormant After UV Disinfection. C&EN News—UV-treated bacteria entered a VBNC state and later revived (Bacteria-Remain-Dormant-UV-Disinfection)
9. González et al. (2023). UV Disinfection Systems & Reactivation of Microorganisms. Sustainability, 15(14)—UV can leave survivors with intact enzymes; bacteria like E. coli repaired UV damage (photoreactivation) (UV Disinfection Systems for Wastewater Treatment: Emphasis on Reactivation of Microorganisms)
10. U.S. EPA (2024). UV Lamps and Indoor Mold—Notes limited effectiveness of home UVGI units; dead mold spores still cause allergies and high UV doses are required for spores/viruses (Does ultraviolet (UV) radiation from UV lamps kill mold?|US EPA).
11. 4. Austin Air (2023). Dangers of UVGI (EPA Review)—Some bacterial and mold spores resist UV; typical residential UV units likely don't fully inactivate them (The Dangers of UV and UVGI—Austin Air Systems. Clinically Proven Air Purifiers.)
12. Shedding Light on UV-C in Hospitals (2023)—Review of UV disinfection tech: highlights shadowing, high contamination, and microbial repair mechanisms as factors that reduce efficacy (Shedding a light on ultraviolet-C technologies in the hospital environment—PMC). Recommends treating UV as a supplement for low-level disinfection (Shedding a light on ultraviolet-C technologies in the hospital environment—PMC).
13. DoD Anthrax Lab Incident Review (2015)—Found that a gamma/UV-irradiated anthrax spore batch was “not fully inactivated and contained viable spores”, affecting dozens of labs ( ). Noted anthrax spores are hard to kill and can repair radiation damage over time ( ).
14. Endotoxin Exposure and Respiratory Health—Research indicates inhaled endotoxin (from bacterial debris) induces lung inflammation even at low levels (Respiratory health effects of exposure to low levels of airborne endotoxin—a systematic review—PMC), underscoring that non-infectious bacterial components can still cause illness.
15. Xia et al., MedComm (2025)—review on respiratory mucosal vaccines (mechanisms of mucosal immunity and vaccine strategies) (How to assess the effectiveness of nasal influenza vaccines? Role and measurement of sIgA in mucosal secretions—PMC) (Novel vaccine strategies to induce respiratory mucosal immunity: advances and implications—PMC).
16. Dhere et al., NEJM (2015)—trial of aerosolized measles vaccine vs. injection in Indian infants (immunogenicity and safety results) (A randomized, controlled trial of an aerosolized vaccine against measles—PubMed) (A randomized, controlled trial of an aerosolized vaccine against measles—PubMed).
17. Satti et al., Lancet Infect Dis (2014)—Phase 1 trial of aerosol TB vaccine MVA85A in adults (lung immune responses vs intradermal) (Safety and immunogenicity of a candidate tuberculosis vaccine MVA85A delivered by aerosol in BCG-vaccinated healthy adults: a phase 1, double-blind, randomised controlled trial—PubMed) (Safety and immunogenicity of a candidate tuberculosis vaccine MVA85A delivered by aerosol in BCG-vaccinated healthy adults: a phase 1, double-blind, randomised controlled trial—PubMed).
18. Fredsgaard-Jones et al., Front. Immunol. (2024)—aerosol BCG in humans (safety up to 10∂CFU and immune response findings) (Frontiers|A dose escalation study to evaluate the safety of an aerosol BCG infection in previously BCG-vaccinated healthy human UK adults) (Frontiers|A dose escalation study to evaluate the safety of an aerosol BCG infection in previously BCG-vaccinated healthy human UK adults).
19. Song et al., J. Med. Virology (2024)—meta-analysis of inhaled COVID-19 vaccines (safety and booster immunogenicity) (Safety, immunogenicity, and protective effective of inhaled COVID-19 vaccines: A systematic review and meta-analysis—PubMed) (Safety, immunogenicity, and protective effective of inhaled COVID-19 vaccines: A systematic review and meta-analysis—PubMed).
20. Zhang et al., Front. Immunol. (2023)—COVID-19 booster trial comparing inhaled vs injected boosters (neutralizing antibodies, T cells, IgA results) (Frontiers|Boosting with an aerosolized Ad5-nCoV elicited robust immune responses in inactivated COVID-19 vaccines recipients).
21. Carreno et al., Organoids (2024)—study using human nasal organoids to test neutralization by vaccine-induced antibodies (novel immunogenicity assay) (Human Nasal Epithelium Organoids for Assessing Neutralizing Antibodies to a Protective SARS-CoV-2 Virus-like Particle Vaccine) (Human Nasal Epithelium Organoids for Assessing Neutralizing Antibodies to a Protective SARS-CoV-2 Virus-like Particle Vaccine).
22. Suzuki et al., Cell Reports Med (2022)—intranasal COVID vaccine in mice with CpG adjuvant (mucosal IgA, T cell responses, reduced lung pathology) (Intranasal vaccination induced cross-protective secretory IgA antibodies against SARS-CoV-2 variants with reducing the potential risk of lung eosinophilic immunopathology—PMC) (Intranasal vaccination induced cross-protective secretory IgA antibodies against SARS-CoV-2 variants with reducing the potential risk of lung eosinophilic immunopathology—PMC).
23. Ambrose et al., Clin. Vaccine Immunol. (2012)—review on LAIV immune response (efficacy in children, correlation with IgA) (Immune responses after live attenuated influenza vaccination—PMC) (How to assess the effectiveness of nasal influenza vaccines? Role . . . ).
24. Forrest et al., NPJ Vaccines (2020)—general overview of nasal vaccine developments (various pathogens and strategies) (Immune responses after live attenuated influenza vaccination—PMC) (iNCOVACC—Intranasal Vaccine For Covid-19—Bharat Biotech).
25. CDC MMWR—Combination Vaccines for Childhood Immunization (1999)—Advantages and considerations of combination vaccines.
26. Liu et al., Vaccines (2022)—Systematic review on immunogenicity and safety of combination vaccines (Immunogenicity and Safety of Childhood Combination Vaccines: A Systematic Review and Meta-Analysis) (Immunogenicity and Safety of Childhood Combination Vaccines: A Systematic Review and Meta-Analysis).
27. Elliman & Bedford, B M J (2003)—Review on efficacy and safety of combination vaccines (Safety and efficacy of combination vaccines: Combinations reduce distress and are efficacious and safe—PMC).
28. Offit et al., Pediatrics (2002)—Immune system capacity and response to multiple vaccines (Safety and efficacy of combination vaccines: Combinations reduce distress and are efficacious and safe—PMC).
29. Eskola, J. Infect. Dis. (1996)—Study on DTP-Hib combination immune response (Analysis of Haemophilus influenzae type B conjugate and diphtheria-tetanus-pertussis combination vaccines—PubMed).
30. CDC Pneumococcal Vaccine Provider Facts—PCV13 vs. PCV7 immunogenicity and impact (About Pneumococcal Vaccines: For Providers|CDC) (About Pneumococcal Vaccines: For Providers|CDC).
31. Yang & Bracken, Can Fam Physician (2016)—Overview of 9-valent HPV vaccine efficacy and safety (Update on the new 9-valent vaccine for human papillomavirus prevention—PMC) (Update on the new 9-valent vaccine for human papillomavirus prevention—PMC).
32. Gamaleya Center (Burtseva et al.), Vaccines (2023)—Combined influenza/COVID mRNA vaccine in mice (no immune interference) (Immunogenicity and Efficacy of Combined mRNA Vaccine Against Influenza and SARS-CoV-2 in Mice Animal Models—PMC).
33. Miller et al., Vaccine (2021)—Febrile seizure risk with MMRV vs. separate MMR+varicella (Do Combination Vaccines or Simultaneous Vaccination Increase the Risk of Adverse Events?—Institute for Vaccine Safety).
34. Global Polio Eradication Initiative—OPV factsheet—Monovalent vs trivalent OPV immunogenicity (The Immunogenicity of Monovalent Oral Poliovirus Vaccine Type 1 (mOPV1) and Inactivated Poliovirus Vaccine (IPV) in the EPI Schedule of India—PMC).
35. Jearanaiwitayakul T, Sunintaboon P, Kittiayuwat A, et al. Intranasal immunization with the bivalent SARS-CoV-2 vaccine effectively protects mice from nasal infection and completely inhibits disease development. Vaccine. 2024; 42(17):3664-3673.
36. J. Xi et al., MedComm. 2021; 2:569-586.
37. Mi, Hongfei et al., eClinicalMedicine, 2024, Volume 67, 102374.
38. Cooper et al., Current Drug Metabolism, 13 (2012), pp. 457-473.
39. Siddiqui et al., Clinical and Experimental Allergy, 38 (2008), pp. 1381-1390.
40. Brain et al., Environmental Research, 11 (1976), pp. 13-33.
41. Liu et al., Toxicology, 304 (2013), pp. 158-166.
42. Bivas-Benita, Zwier, Junginger, & Borchard, European Journal of Pharmaceutics and Biopharmaceutics, 61 (2005), pp. 214-218.
43. Guillon et al., International Journal of Pharmaceutics, 434 (2012), pp. 481-487
44. Morello et al., Tuberculosis (Edinburgh, Scotland), 89 (2009), pp. 371-377
45. Pauluhn & Mohr, Toxicologic Pathology, 28 (2000), pp. 734-753
46. J. E. Phillips, Pharmacology & Therapeutics, volume 178, October 2017, Pages 141-147.
47. De Angelis M L, Francescangeli F, Rossi R, Giuliani A, De Maria R, Zeuner A. Repeated Exposure to Subinfectious Doses of SARS-CoV-2 May Promote T Cell Immunity and Protection against Severe COVID-19. Viruses. 2021 May 22; 13(6):961.
48. Wim Van Damme et al., Medical Hypotheses, Volume 146, 2021, Article 110431.
49. Karimzadeh S, Bhopal R, Nguyen Tien H. Review of infective dose, routes of transmission and outcome of COVID-19 caused by the SARS-COV-2: comparison with other respiratory viruses. Epidemiol Infect. 2021 Apr. 14; 149:e96.
50. Song B M, Lee G H, Han H J, Yang J H, Lee E G, Gu H, Park H K, Ryu K, Kim J, Kang S M, Tark D. Ultraviolet-C light at 222 nm has a high disinfecting spectrum in environments contaminated by infectious pathogens, including SARS-CoV-2. PLoS One. 2023 Nov. 28; 18(11):e0294427.
51. Eadie E, Hiwar W, Fletcher L, Tidswell E, O'Mahoney P, Buonanno M, Welch D, Adamson C S, Brenner D J, Noakes C, Wood K. Far-UVC (222 nm) efficiently inactivates an airborne pathogen in a room-sized chamber. Sci Rep. 2022 Mar. 23; 12(1):4373.

1.-138. (canceled)

139. A method of triggering an immune response in a subject, the method comprising:
exposing a volume of air, a volume of liquid, a volume of vapors, and/or a surface area to an ultraviolet light to generate an immunogenic composition; and
exposing the subject to the immunogenic composition.

140. The method according to claim 139, wherein the ultraviolet light comprises UVA, UVB, UVC, or any combination thereof.

141. The method according to claim 140, wherein the ultraviolet light comprises UVC.

142. The method according to claim 141, wherein the ultraviolet light comprises an output wavelength of from about 100 nm to about 280 nm.

143. The method according to claim 142, wherein the ultraviolet light comprises far-UVC.

144. The method according to claim 143, wherein the ultraviolet light comprises an output wavelength of from about 200 nm to about 230 nm.

145. The method according to claim 144, wherein the ultraviolet light comprises an output wavelength of about 222 nm.

146. The method according to claim 139, wherein the ultraviolet light is emitted from an ultraviolet system, and wherein the ultraviolet system is configured to adjust an intensity or a targeted area of the ultraviolet light based on sensor data indicating the presence of pathogens in the volume of air, volume of liquid, volume of vapors, and/or surface area.

147. The method according to claim 146, wherein the intensity or the targeted area of the ultraviolet light is adjusted to alter the immunogenic composition.

148. The method according to claim 139, wherein the volume of air, the volume of liquid, the volume of vapors, and/or the surface area comprises a suspended liquid particle, a respiratory droplet, a particulate matter, a pathogen, or a plurality of different pathogens.

149. The method according to claim 148, wherein the pathogen is a microbe, bacteria, virus, mold, parasite, allergen, fungi, protozoa, or prion.

150. The method according to claim 148, wherein the immunogenic composition comprises at least from 10⁸ to 10¹¹ inactivated, attenuated, weakened, dormant, or killed virus or particles thereof.

151. The method according to claim 148, wherein the pathogen or the plurality of different pathogens are inactivated, attenuated, weakened, dormant, or killed after exposure to the ultraviolet light.

152. The method according to claim 151, wherein the dormant pathogen or plurality of different pathogens are viable but not infectious or not culturable.

153. The method according to claim 152, wherein the inactivated pathogen or plurality of different pathogens are an aerosolized inactivated pathogen or an aerosolized inactivated plurality of different pathogens.

154. The method according to claim 153, wherein the inactivated, attenuated, weakened, dormant, or killed pathogen or plurality of different pathogens comprises one or more epitopes for generating an immune response.

155. The method according to claim 154, wherein the immune response is against the pathogen or the plurality of different pathogens.

156. The method according to claim 139, wherein exposing the subject to the immunogenic composition comprises contacting to skin, via inhalation, via intranasal, contacting to a membrane, or any combination thereof.

157. The method according to claim 139, wherein the subject comprises an immune system.

158. The method according to claim 139, wherein the method comprises preventing, treating, or reducing a disease or condition in the subject, wherein the disease or condition is associated with or resulting from a microbe.
